(19) United States
(12) Patent Application Publication
(10) Pub. No.: US 2023/0202996 A1
(43) Pub. Date: Jun. 29, 2023

(54) SYNTHETIC METHODS FOR MAKING CARBOXYLIC ACIDS, ESTERS AND LACTONES

(71) Applicant: P2 SCIENCE, INC., Woodbridge, CT (US)

(72) Inventors: Yong TU, Cheshire, CT (US); Yonghua YANG, East Lyme, CT (US); Patrick FOLEY, New Haven, CT (US); Ted ANASTASIOU, New Haven, CT (US); Tania SALAM, New Haven, CT (US); Alex MATTESON, Woodbridge, CT (US); Vivek BULBULE, Woodbridge, CT (US)

(21) Appl. No.: 18/145,383

(22) Filed: Dec. 22, 2022

Related U.S. Application Data
(60) Provisional application No. 63/343,897, filed on May 19, 2022.
(60) Provisional application No. 63/293,483, filed on Dec. 23, 2021.

(51) Int. Cl. C07D 307/83; B01J 23/30; B01J 21/16

(52) U.S. Cl. CPC C07D 307/83; B01J 23/30; B01J 21/16

(57) ABSTRACT

The present disclosure pertains to a new synthetic method for the preparation of 3,6-dimethylhexahydrobenzofuran-2-one, a derivative of mint lactone, and an important organoleptic compound which finds use in the flavor and fragrance industries. Applicants' novel synthetic route is also applicable to other alkene compounds.

### CROSS-REFERENCE TO RELATED APPLICATIONS

This application is non-provisional application which claims priority to U.S. Provisional Applications Ser. No. 63/293,483, filed on Dec. 23, 2021, and 63/343,897, filed on May 19, 2022, the contents of each of which are hereby incorporated by reference in their entireties.

### FIELD OF INVENTION

The present disclosure pertains to a new synthetic method for the preparation of 3,6-dimethylhexahydrobenzofuran-2-one, a derivative of mint lactone, and an important organoleptic compound which finds use in the flavor and fragrance industries. Applicants' novel synthetic route is also applicable to other alkene compounds.

### BACKGROUND

Mint lactone is a natural component of mint oil, and has been used as a precursor to 3,6-dimethylhexahydrobenzofuran-2-one (also known as Koumalactone®), a valued flavor and fragrance ingredient.

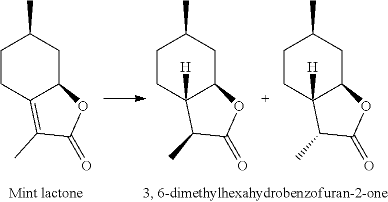

Multiple efforts have been made to cost effectively produce mint lactone, and from it, 3,6-dimethylhexahydrobenzofuran-2-one. Koch (U.S. Pat. No. 6,512,126) describe a hydrogenation and elimination of hydroxymenthofurolactone (I):

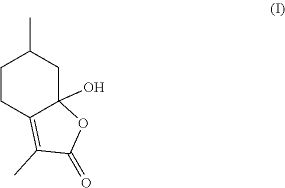

Xiong (CN 102,850,309) describes the treatment of 3-methylcyclohexanone (II), with methyl pyruvate in a multi-step synthesis involving sodium borohydride and iron chloride:

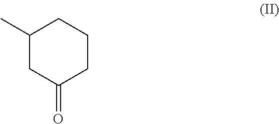

While both of these approaches are practicable, they use relatively expensive starting materials and reagents (e.g., Pd/C, NaBH₄). Another major drawback to these methods is that these methods do not produce highly enantio-enriched material. Additional approaches, including the use of citronellal (Shishido, et al., Tetrahedron Letters, 33(32), 4589-4592 (1992)), and alkynyl aldehydes (Gao et al., Journal of Organic Chemistry, 74(6), 2592 (2009)), have also been described, but again lack economic feasibility.

The naturally occurring, and commercially available, compound isopulegol (III), has been used as a key precursor in the synthesis of mint lactone and 3,6-dimethylhexahydrobenzofuran-2-one:

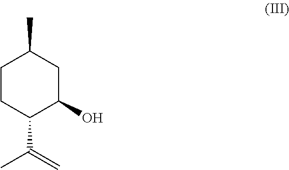

Although isopulegol (III) has been used as a starting material for the synthesis of enantiopure mint lactone in the past (Chavan et al., Tetrahedron Letters, 49(29), 6429-6436 (1993)), the approaches appear to have the common problem of being too costly to be commercially attractive, including the use of hydroboration and deprotonation with lithium diisopropylamide under cryogenic conditions.

As a result of these limitations, mint lactone, and its derivative 3,6-dimethylhexahydrobenzofuran-2-one, have been very expensive to obtain commercially, especially with the desired stereochemistry, and therefore their use has been limited.

Additionally, specific isomers of 3,6-dimethylhexahydrobenzofuran-2-one have been difficult to obtain using traditional routes. Gaudin, Tetrahedron Letters 56(27), 4769-4776 (2000) and Gaudin, U.S. Pat. No. 5,464,824, report a route starting from isopulegol requiring double bond epoxidation, ring opening with lithium diisopropylamide to give isomeric allylic diols, followed by hydrogenation to form isomeric menthanediols, and then oxidation to form the lactone rings. Other similar routes are disclosed in Chinese patent application CN112010826A and U.S. Pat. No. 10,995,080.

U.S. Pat. Nos. 10,399,954, and 11,008,299, described improved methods of creating mint lactone and 3,6-dimethylhexahydrobenzofuran-2-one from isopulegol, in a manner which permits one to easily obtain the desired natural stereochemistry while using inexpensive and commercially available reagents. According to this method, isopulegol (III) is treated with ozone to cleave the double bond, followed by reductive quenching with sodium bisulfite to remove peroxide and generate 1-(2-hydroxy-4-methyl-cyclohexyl)ethanone (IV). This hydroxy ketone (IV) is then treated with a solution of aqueous sodium cyanide to generate a cyanohydrin intermediate that is hydrolyzed in the presence of a strong aqueous acid to generate an alpha-hydroxylactone (V):

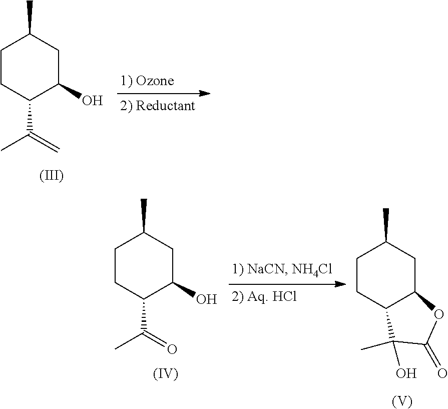

Compound (V) is then treated with known conditions to eliminate the alcohol to generate enantiopure mint lactone (VI) (e.g., Shishido et al., Tetrahedron Letters, 33(32), 4589-4592 (1992)), and subsequently hydrogenated to generate enantio-enriched 3,6-dimethylhexahydrobenzofuran-2-one (VII):

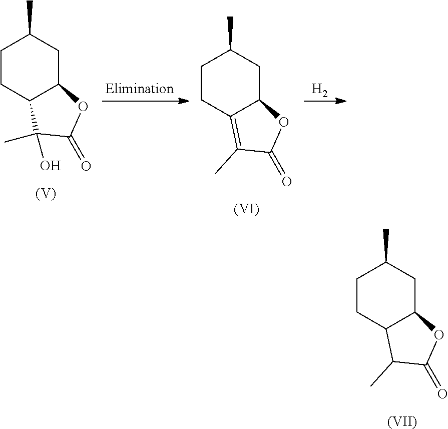

However, it has been found that reduction of mint lactone (VI) under basic hydrogenation conditions generates a mixture of isomeric hydrogenation products (IX), (X), (XI) and (XII):

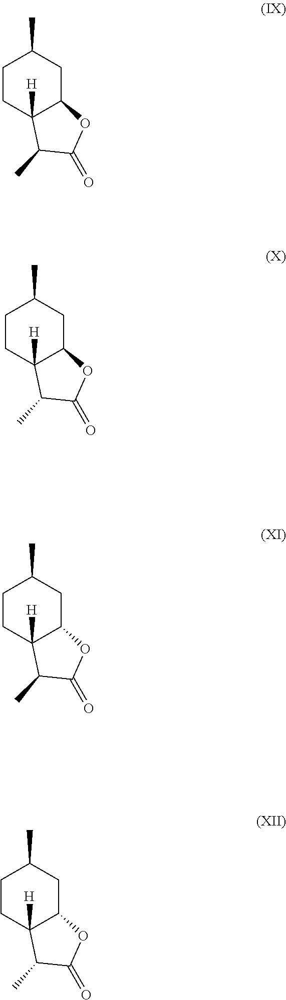

Alternatively, compound (V) can also be deoxygenated via halogenation (X=Cl, Br, I) to form alpha-halo lactone compound (VIII), and then reduced to form enantio-enriched 3,6-dimethylhexahydrobenzofuran-2-one (VII). For example, compound (V) can be halogenated using a reagent such as thionyl chloride, PCl₃, POCl₃, HCl, cyanuric chloride, PCl₅, SO₂Cl₂, CCl₄, PBr₃, HBr, HI, or any other suitable halogenating agent. The halide intermediate can then be reduced using catalytic hydrogenation in the presence of a catalyst (e.g., Pd, Ru, Ni, Rh, Cu), or through electrolysis, or through treatment with Zn in a suitable acid, such as acetic acid. Importantly, these methods result in only two isomers of (VII), in particular, the diastereomers (IX) and (X).

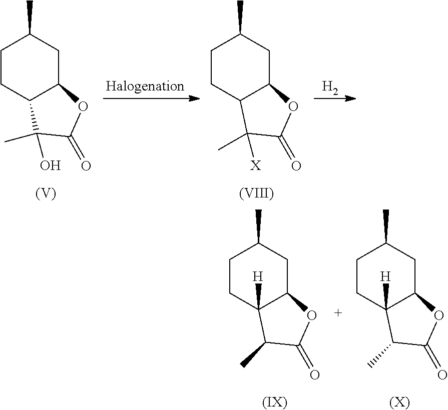

The compounds (IX) and (X), which retain the stereochemistry present in the mint lactone precursor, are highly sought-after fragrance materials that possess a very powerful lactonic and coumarinic type odor. Halogenation and hydrogenation, however, are expensive to carry out on an industrial scale, requiring either specialized equipment, special safety precautions due to reagent toxicity, or costs associated with environmental protection or hazardous waste remediation.

Therefore, there remains a need for improved methods for the synthesis of 3,6-dimethylhexahydrobenzofuran-2-one, particularly isomers (IX) and (X), which rely on less hazardous, less costly and/or less toxic reagents, as well as a need for obtaining the highest yields using the least expensive starting materials.

### BRIEF SUMMARY

The inventors have discovered a much-improved, economically feasible and relatively safe method for the synthesis of carboxylic acids from alkenes without the loss of a carbon atom, comprising the epoxidation of the alkene, rearrangement of the epoxide to an aldehyde, and oxidation of the aldehyde to a carboxylic acid.

The method may be particularly advantageously applied to the synthesis of 3,6-dimethylhexahydrobenzofuran-2-one. The present disclosure provides a method of making 3,6-dimethylhexahydrobenzofuran-2-one comprising the epoxidation of isopulegol, or a derivative thereof, rearrangement to an aldehyde, oxidation to the carboxylic acid, and internal ring closure to form 3,6-dimethylhexahydrobenzofuran-2-one. The present disclosure further provides one-pot methods for carrying out the same transformations. The present disclosure further provides one-pot methods for carrying out the same transformations on other alkene compounds.

### DETAILED DESCRIPTION

Applicants have discovered a much-improved, economically feasible and relatively safe method for the synthesis of 3,6-dimethylhexahydrobenzofuran-2-one. The method involves the use of relatively inexpensive and safe reagents compared to prior art methods, and results in good yields for high cost-effectiveness.

In a first aspect, the present disclosure therefore provides, a method (Method 1) of making 3,6-dimethylhexahydrobenzofuran-2-one (Compound 1) comprising the steps of:
(A) epoxidizing isopulegol or a derivative thereof (Compound 2), to form epoxide Compound (3);
(B) rearranging epoxide Compound (3) to form aldehyde Compound (4);
(C) oxidizing aldehyde Compound (4) to form carboxylic acid Compound (5); and
(D) ring closing the carboxylic acid Compound (5) to form 3,6-dimethylhexahydrobenzofuran-2-one (Compound 1):

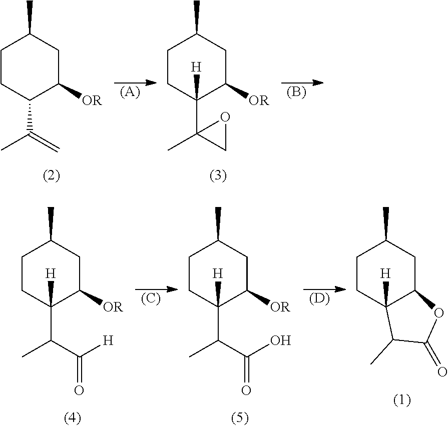

wherein R is H or a protecting group (e.g., an ether, an ester, or a silyl ether protecting group). Importantly, the critical stereochemistry around the cyclohexane ring is retained during this synthetic method.

In further embodiments of the first aspect, the present disclosure provides:
1.1 Method 1, wherein R is H;
1.2 Method 1, wherein R is an ester protecting group, e.g., R is —C(O)—R¹ or —S(O)₂—R¹, and wherein R¹ is H, C₁₋₆alkyl (e.g., methyl or ethyl), haloC₁₋₆alkyl (e.g., chloromethyl or trifluoromethyl), C₁₋₆alkoxy (e.g., methoxy or ethoxy), C₁₋₆alkoxymethyl (e.g., methoxyethyl or ethoxymethyl), aryl (e.g., phenyl or tolyl), arylmethyl (e.g., benzyl), aryloxy (e.g., phenoxy), or aryloxymethyl (e.g., phenoxymethyl);
1.3 Method 1.2, wherein R is —C(O)—R¹, and wherein R¹ is methyl, ethyl, propyl, isopropyl, or tert-butyl, or wherein R is —S(O)₂—R¹, and wherein R¹ is methyl;
1.4 Method 1.2, wherein R is —C(O)—R¹, and wherein R¹ is methyl;
1.5 Method 1.2, wherein R is —C(O)—R¹, and wherein R¹ is chloromethyl, dichloromethyl, trichloromethyl, 2,2,2-trichloroethyl, trifluoromethyl, methoxymethyl, phenoxymethyl, or benzyl, or wherein R is —S(O)₂—R¹, and wherein R¹ is trifluoromethyl, phenyl or tolyl;
1.6 Method 1, wherein R is an ether protecting group, e.g., R is unsubstituted C₁₋₆alkyl (e.g., methyl, ethyl, isopropyl, tert-butyl), or substituted C₁₋₆alkyl, such as C₁₋₆alkoxy-C₁₋₆alkyl, aryloxy-C₁₋₆alkyl, or aryl-C₁₋₆alkyl (e.g., —CH₂-O-Me, —CH₂-O-Et, —CH₂-S-Me, —CH₂—O—CH₂CH₂—OMe, CH₂—O—CH₂CCl₃, CH₂—O—CH₂CH₂—SiMe₃, —CH₂—O-Ph, —CH₂—O—CH₂-Ph, —CH₂—O—CH₂-(4-methoxyphenyl), —CH₂—O—CH₂-(3,4-dimethoxyphenyl), —CH₂CH₂-OEt, —CH₂CH₂Si(Me)₃, —CH₂CCl₃, —CH₂-Ph, —CH₂-(4-methoxyphenyl), —CH₂-(3,4-dimethoxyphenyl), —CH₂-(2,6-dimethoxyphenyl), or 2-tetrahydropyranyl;
1.7 Method 1, wherein R is a silyl ether protecting group, e.g., R is —Si(R²)(R³)(R⁴), wherein R², R³, and R⁴ are each independently selected from C₁₋₆alkyl (e.g., methyl, ethyl, isopropyl, tert-butyl, thexyl, benzyl), C₁₋₆alkoxy (e.g., methoxy, ethoxy, tert-butoxy), and aryl (e.g., phenyl);
1.8 Method 1.7, wherein R is selected from trimethylsilyl, triethylsilyl, triisopropylsilyl, dimethylisopropylsilyl, diethylisopropylsilyl, dimethylthexylsilyl, tert-butyldimethylsilyl, tert-butyldiphenylsilyl, tribenzylsilyl, triphenylsilyl, diphenylmethylsilyl, di-tert-butylmethylsilyl, tris(trimethylsilyl)silyl, tert-butylmethoxyphenylsilyl, and tert-butoxydiphenylsilyl;
1.9 Method 1 or any of 1.1 to 1.8, wherein the Compound (3) is Compound (3a), Compound (3b) or a mixture thereof:

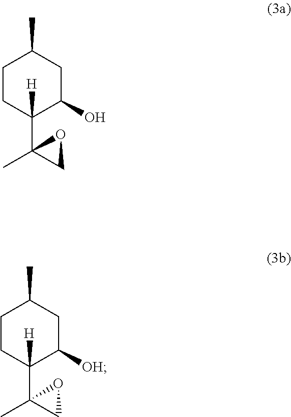

1.10 Method 1 or any of 1.1 to 1.9, wherein the Compound (4) is Compound (4a), Compound (4b) or a mixture thereof:

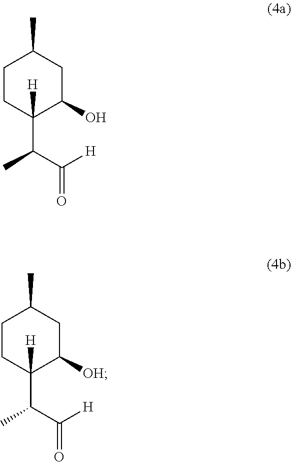

1.11 Method 1 or any of 1.1 to 1.10, wherein the Compound (5) is Compound (5a), Compound (5b) or a mixture thereof:

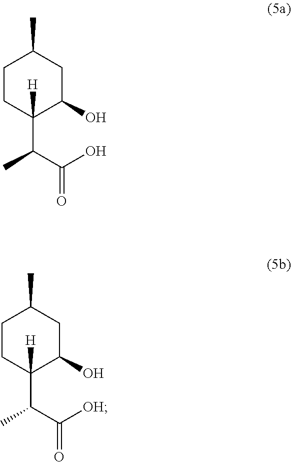

1.12 Method 1 or any of 1.1 to 1.11, wherein the Compound (1) is Compound (1a), Compound (1b) or a mixture thereof:

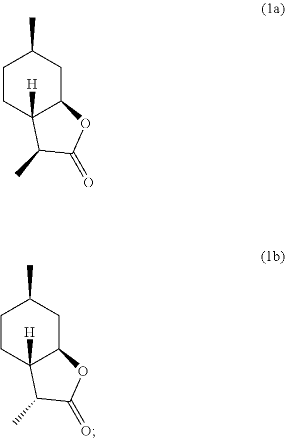

optionally, wherein the Compound (1) is enriched in one isomer or the other isomer, or wherein the method further comprises the step of purifying or separating the isomers;
1.13 Method 1, or any of 1.1-1.12, further comprising the step (A′) of converting Compound (2′), wherein R is H, to Compound (2″), wherein R is not H (e.g., wherein R is a protecting group):

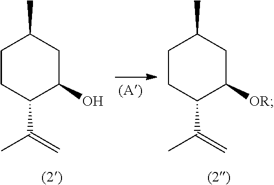

1.14 Method 1.13, wherein step (A′) is the first step in method, immediately preceding step (A);
1.15 Method 1, or any of 1.1-1.14, further comprising the step (D′) of converting Compound (5′), wherein R is not H (e.g., wherein R is a protecting group), to Compound (5″), wherein R is H:

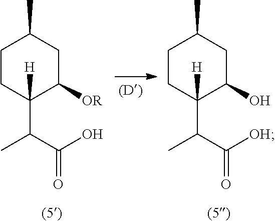

1.16 Method 1.15, wherein step (D′) is the penultimate step in method, immediately preceding step (D);
1.17 Method 1, or any of 1.1-1.16, wherein the protecting group R is eliminated during step (D) (i.e., no deprotection step D′ is needed);
1.18 Method 1, or any of 1.1-1.17, wherein the method does not comprise any step using mint lactone (compound (VI) hereinabove) as an intermediate;
1.19 Method 1, or any of 1.1-1.18, wherein the method does not comprise any step using compound (IV), compound (V), or compound (VIII), as an intermediate

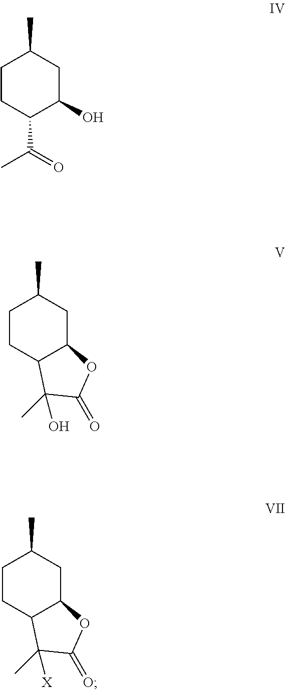

wherein X is Cl, Br, or I;
1.20 Method 1, or any of 1.1-1.19, wherein the method does not yield any measurable amount of compound (XI) or compound (XII), as described hereinabove, e.g., as measured by HPLC, GC, MS, or NMR;
1.21 Method 1, or any of 1.1-1.20, wherein the method does not comprise any synthetic steps and/or mechanistic steps other than steps (A), (B), (C), and/or (D), and optionally (A′) and/or (D′) from Compound (2) or Compound (2′) through Compound (1);
1.22 Method 1, or any of 1.1-1.21, wherein epoxidation step (A) is carried out by treating the compound (2) with a suitable oxidizing agent in a suitable solvent;
1.23 Method 1.22, wherein the suitable oxidizing agent is one or more of hydrogen peroxide, osmium tetroxide, peracetic acid, perchloric acid, perbenzoic acid, meta-chloroperoxybenzoic acid (mCPBA), trifluoroperacetic acid, magnesium monoperoxyphthalate, dimethyl dioxirane (DMDO), tert-butyl hydroperoxide, sodium hypochlorite, sodium tungstate, sodium periodate, iodosyl benzene, pentafluoroiodosyl benzene, cumene hydroperoxide, potassium persulfate, potassium monoperoxysulfate, pyridine N-oxide, 2,6-dichloropyridine N-oxide, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries);
1.24 Method 1.22, wherein in epoxidation step (A) the suitable oxidizing agent is hydrogen peroxide, peracetic acid, meta-chloroperoxybenzoic acid, tert-butyl hydroperoxide, or potassium monoperoxysulfate;
1.25 Method 1.22, wherein in epoxidation step (A) the suitable oxidizing agent is hydrogen peroxide and sodium tungstate, e.g., hydrogen peroxide (e.g., 30 wt. %, e.g., 1-1.5 equivalents) plus sodium tungstate (e.g., sodium tungstate dihydrate, e.g., 0.01-0.10 equivalents) with methyl-tri-n-octylammonium hydrogen sulfate (e.g., 0.01-0.05 equivalents) and phenylphosphonic acid (e.g., 0.01-0.05 equivalents), optionally at 0-50° C., optionally in aqueous solvent;
1.26 Method 1.22, wherein in epoxidation step (A) the suitable oxidizing agent is m-chloroperoxybenzoic acid;
1.27 Method 1.22, wherein in epoxidation step (A) the suitable oxidizing agent is oxygen gas;
1.28 Method 1.27, wherein in epoxidation step (A) the suitable oxidizing agent is oxygen gas with a transition metal catalyst;
1.29 Method 1.27, wherein in epoxidation step (A) the suitable oxidizing agent is oxygen gas and iron(III)-tetraphenyl porphyrin complex (Fe(III)TPP), in the presence of a C₂₋₁₀ aliphatic aldehyde (e.g., isobutyraldehyde);
1.30 Method 1.27, wherein in epoxidation step (A) the suitable oxidizing agent is oxygen gas and N-hydroxyphthalimide;
1.31 Any of Methods 1.22-1.30, wherein in epoxidation step (A) does not comprise the use of ozone;
1.32 Any of Methods 1.22-1.31, wherein in epoxidation step (A) the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
1.33 Any of Methods 1.22-1.32, wherein the epoxidation step (A) is carried out using
1.0 to 5.0 equivalents of the oxidizing agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents; and optionally 0.01 to 1.0 equivalents of any one or more additional reagents (e.g., secondary oxidant, or catalyst, or ligand, or other agent), e.g., 0.01 to 0.5 equivalents, 0.01 to 0.2 equivalents, or 0.01 to 0.1, or 0.01 to 0.05 equivalents;
1.34 Any of Methods 1.22-1.33, wherein the epoxidation step (A) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
1.35 Any of Methods 1.22-1.34, wherein the epoxidation step (A) is carried out in a batch reactor;
1.36 Any of Methods 1.22-1.34, wherein the epoxidation step (A) is carried out in a continuous flow reactor;
1.37 Method 1, or any of 1.1-1.36, wherein the rearrangement step (B) is carried out by treating the compound (3) with a suitable rearrangement catalyst in a suitable solvent, or by heating the compound (3) without catalyst in a suitable solvent (i.e., thermal rearrangement);
1.38 Method 1.37, wherein the rearrangement catalyst is a Lewis acid, a Bronsted acid, a strong base (e.g., LDA, LiTMP, LiHMDS, t-butyl lithium), or a transition metal catalyst or complex (e.g., palladium, ruthenium, rhodium, chromium, iridium, zirconium, manganese, iron, or nickel catalyst or complex);
1.39 Method 1.37, wherein the rearrangement catalyst is a solid phase acidic resin (e.g., an acidic polymer resin such as Amberlyst or Nafion-H, or a Montmorillonite, or a Zeolite), e.g., Montmorillonite K10, or Amberlyst H-15, optionally wherein the catalyst is Montmorillonite K-10, e.g., at 0.1-0.5 equivalents, e.g., in toluene solvent, e.g., at 0-50° C. (e.g., about 25° C.);
1.40 Method 1.37, wherein the rearrangement catalyst is a Lewis acid, e.g., selected from zinc bromide, zinc chloride, magnesium bromide, magnesium bromide-diethyl ether complex, bismuth triflate, boron trifluoride-diethyl ether complex, aluminum triisopropoxide, titanium tetraisopropoxide, titanium tetrachloride, iron trichloride, indium chloride, lithium perchlorate, iridium chloride, lithium bromide, borane-THF complex, chromium tetraphenyl porphyrin triflate, nickel bis(triphenylphosphine) dibromide complex, methyl bis(4-bromo-1,6-di-tert-butylphenoxy) aluminum;
1.41 Method 1.37, wherein the rearrangement catalyst is a Bronsted acid, e.g., selected from hydrochloric acid, perchloric acid, sulfuric acid, phosphoric acid, acetic acid, peracetic acid, trifluoromethanesulfonic acid, methanesulfonic acid, trifluoroacetic acid, and nitric acid, or a heteropoly acid (e.g., phosphotungstic acid);
1.42 Any of Methods 1.37-1.41, wherein in rearrangement step (B) the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
1.43 Any of Methods 1.37-1.42, wherein rearrangement step (B) is carried out using
1.0 to 5.0 equivalents of the rearrangement catalyst, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents; or using 0.01 to 1.0 equivalents of the rearrangement catalyst, e.g., 0.01 to 0.1 equivalents, 0.1 to 0.5 equivalents, or 0.5 to
1.0 equivalents;
1.44 Any of Methods 1.37-1.43, wherein rearrangement step (B) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
1.45 Any of Methods 1.37-1.44, wherein rearrangement step (B) is carried out in a batch reactor;
1.46 Any of Methods 1.37-1.45, wherein rearrangement step (B) is carried out in a continuous flow reactor;
1.47 Method 1, or any of 1.1-1.46, wherein rearrangement step (B) is carried out in the same vessel as oxidation step (C), e.g., wherein the product of step (B) is not purified or isolated before carrying out step (C), or wherein the reagent or reagents and solvent for step (C) is added directly to the reaction mixture of step (B);
1.48 Method 1, or any of 1.1-1.47, wherein oxidation step (C) is carried out by treating the compound (4) with a suitable oxidizing agent in a suitable solvent;
1.49 Method 1.48, wherein in oxidation step (C) the suitable oxidizing agent is one or more of a chromium oxidant (e.g., chromium trioxide, chromic acid, pyridinium chlorochromate, potassium dichromate, chromium trioxide-pyridine complex, pyridinium dichromate), osmium tetroxide, potassium permanganate, silver oxide, hydrogen peroxide, peracetic acid, perchloric acid, trifluoroperacetic acid, periodic acid, potassium periodate, sodium chlorite, oxygen and N-hydroxyphthalimide, potassium persulfate, and potassium monoperoxysulfate;
1.50 Method 1.48, wherein in oxidation step (C) the suitable oxidizing agent is a chromium oxidant (e.g., chromium trioxide, chromic acid, pyridinium chlorochromate, potassium dichromate, chromium trioxide-pyridine complex, pyridinium dichromate), optionally wherein the chromium oxidant is chromium trioxide in aqueous sulfuric acid (i.e., Jones Reagent), (e.g., about 1-1.5 equivalents Jones Reagent, e.g., in acetone solvent, e.g., at 0-50° C. (e.g., about 25° C.);
1.51 Any of Methods 1.48-1.50, wherein in oxidation step (C) does not comprise the use of ozone;
1.52 Any of Methods 1.48-1.51, wherein in oxidation step (C) the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
1.53 Any of Methods 1.48-1.52, wherein the oxidation step (C) is carried out using 1.0 to 5.0 equivalents of the oxidizing agent, 1.0 to 5.0 equivalents of the rearrangement catalyst, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents;
1.54 Any of Methods 1.48-1.53, wherein the oxidation step (C) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
1.55 Any of Methods 1.48-1.54, wherein the oxidation step (C) is carried out in a batch reactor;
1.56 Any of Methods 1.48-1.54, wherein the oxidation step (C) is carried out in a continuous flow reactor;
1.57 Method 1, or any of 1.1-1.56, wherein ring closure step (D) and/or deprotection of the group —OR of the compound (5) occurs spontaneously during and/or after oxidation step (C);
1.58 Method 1, or any of 1.1-1.56, wherein ring closure step (D) and/or deprotection of the group —OR of the compound (5) is carried out by heating the product mixture from step (C);
1.59 Method 1, or any of 1.1-1.56, wherein ring closure step (D) and/or deprotection of the group —OR of the compound (5) is carried out by distilling the product mixture from step (C);
1.60 Method 1, or any of 1.1-1.56, wherein the method comprises deprotection step (D′) immediately prior to ring closure step (D) and immediately after oxidation step (C);
1.61 Method 1.60, wherein the deprotection step (D′) is carried out by treating the compound (5) with aqueous base, aqueous acid, anhydrous base, anhydrous acid, or anhydrous fluoride, or biphasic acid, biphasic base, or biphasic fluoride, or other suitable conditions (e.g., sodium azide, mercuric chloride, magnesium bromide, magnesium iodide, DDQ (2,3-dichloro-5,6-dicyano-1,4-benzoquinone, ceric ammonium nitrate, hydrogen over palladium or platinum catalyst), in a suitable solvent;
1.62 Method 1.61, wherein the base is selected from lithium hydroxide, sodium hydroxide, potassium hydroxide, sodium methoxide, potassium methoxide, potassium tert-butoxide, sodium carbonate, potassium carbonate, cesium carbonate, ammonium hydroxide, tetrabutylammonium hydroxide, ammonia, guanidine, ethylene diamine, ethanolamine, pyridine, lutidine, collidine, triethylamine, diisopropylethylamine, piperidine, morpholine, methylamine, hydrazine, and imidazole;
1.63 Method 1.62, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or combinations thereof (including aqueous or anhydrous or biphasic combinations thereof);
1.64 Method 1.61, wherein the acid is selected from hydrochloric acid (e.g., hydrogen chloride), nitric acid, sulfuric acid, phosphoric acid, acetic acid, peracetic acid, formic acid, citric acid, trifluoroacetic acid, trifluoromethanesulfonic acid, methanesulfonic acid, perchloric acid, hydrogen fluoride, hydrogen bromide, boron trifluoride-etherate, toluenesulfonic acid, scandium triflate, ytterbium triflate, pyridinium para-toluenesulfonate (PPTS), zinc bromide, zinc chloride, titanium chloride, tin(IV) chloride, bromodimethylborane, boron trichloride, pyridine-HF complex, and solid acidic resins (e.g., acidic polymers such as Amberlyst H-15; silica gel, alumina, Montmorillonite K-10);
1.65 Method 1.64, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or combinations thereof (including aqueous or anhydrous or biphasic combinations thereof);
1.66 Method 1.61, wherein the fluoride is selected from hydrogen fluoride (e.g., anhydrous HF, aqueous HF, triethylamine HF complex, pyridine HF complex), TASF (tris(dimethylamino)sulfonium difluorotrimethylsilicate), sodium fluoride, potassium fluoride, cesium fluoride, ammonium fluoride, and tetrabutylammonium fluoride;
1.67 Method 1.66, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid), carbon dioxide (e.g., supercritical carbon dioxide), and carbon disulfide, or combinations thereof (including aqueous or anhydrous or biphasic combinations thereof);
1.68 Any of Methods 1.60-1.67, wherein the deprotection step (D′) is carried out using
1.0 to 5.0 equivalents of the acid, base, fluoride or other agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents;
1.69 Any of Methods 1.60-1.68, wherein the deprotection step (D′) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
1.70 Any of Methods 1.60-1.69, wherein the deprotection step (D′) is carried out in a batch reactor;
1.71 Any of Methods 1.60-1.70, wherein the deprotection step (D′) is carried out in a continuous flow reactor;
1.72 Method 1, or any of 1.1-1.71, further comprising the protection step (A′);
1.73 Method 1.72, wherein protection step (A′) is carried out by treating the compound (2) with a suitable protecting agent in a suitable solvent, optionally with a suitable base;
1.74 Method 1.73, wherein the protecting agent is selected from an acyl halide (e.g., acetyl chloride, benzoyl chloride, chloroacetyl chloride, dichloroacetyl chloride, trichloroacetyl chloride, methoxyacetyl chloride, phenoxyacetyl chloride, pivaloyl chloride, benzoyl chloride), an acyl anhydride (e.g., acetic anhydride, chloroacetic anhydride, dichloroacetic anhydride, trichloroacetic anhydride, trifluoroacetic anhydride, methoxyacetyl anhydride, phenoxyacetyl anhydride, pivaloyl anhydride, benzoyl anhydride), an alkyl halide (e.g., methoxymethyl chloride, methoxymethyl bromide, methoxyethyl chloride, methylthiomethyl iodide, benzyloxymethyl chloride, 4-methoxybenzyloxymethyl chloride, 2-methoxyethoxymethyl chloride, 2,2,2-trichloroethoxymethyl chloride, 2-trimethylsilylethoxymethyl chloride, 4-methoxybenzyl chloride, 4-methoxybenzyl bromide, 3,4-dimethoxybenzyl bromide,), a silyl reagent (e.g., the chloride, silane or triflate of the group trimethylsilyl, triethylsilyl, triisopropylsilyl, dimethylisopropylsilyl, diethylisopropylsilyl, dimethylthexylsilyl, tert-butyldimethylsilyl, tert-butyldiphenylsilyl, tribenzylsilyl, triphenylsilyl, diphenylmethylsilyl, di-tert-butylmethylsilyl, tris(trimethylsilyl)silyl, tert-butylmethoxyphenylsilyl, and tert-butoxydiphenylsilyl), or other protecting agents (e.g., formic acid, acetic acid, ethyl formate, methyl formate, chloroacetic acid, dihydropyran, 2-hydroxytetrahydropyran, ethyl vinyl ether, trimethylsilylethoxyethene, isobutylene, methanesulfonyl chloride, trifluoromethanesulfonyl chloride, trifluoromethanesulfonic anhydride, N,N-bis(trifluoromethanesulfonyl)aniline, benzenesulfonyl chloride, toluenesulfonyl chloride);
1.75 Method 1.73 or 1.74, wherein the suitable base is selected from sodium hydride, potassium hydride, hydroxide bases (e.g., sodium hydroxide, potassium hydroxide), alkoxide bases (e.g., sodium tert-butoxide, potassium tert-butoxide), carbonate bases (e.g., sodium carbonate, potassium carbonate, cesium carbonate), bicarbonate bases (e.g., sodium bicarbonate), and amine bases (e.g., triethylamine, diisopropylethylamine, N-methyl morpholine, DBU, DBN, pyridine, dimethylaminopyridine, imidazole);
1.76 Any of methods 1.73-1.75, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid), carbon dioxide (e.g., supercritical carbon dioxide), and carbon disulfide, or combinations thereof, or wherein the solvent is a neat reagent (e.g., pyridine);
1.77 Any of Methods 1.73-1.76, wherein the protection step (A′) is carried out using
1.0 to 5.0 equivalents of the protecting agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents;
1.78 Any of Methods 1.73-1.77, wherein the protection step (A′) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
1.79 Any of Methods 1.73-1.78, wherein the protection step (A′) is carried out in a batch reactor;
1.80 Any of Methods 1.73-1.79, wherein the protection step (A′) is carried out in a continuous flow reactor;
1.81 Any of Methods 1.1-1.80, wherein the intermediates (3), (4), and (5), of steps (A), (B), (C), and (D) are not isolated, e.g., wherein the reactant Compound (2) proceeds to the product Compound (1) in a single vessel;
1.82 Method 1.81, wherein the reaction comprises the treatment of Compound (2) with an oxidizing agent, as hereinbefore described (e.g., an oxidizing agent suitable for step (A) or for step (C)), and an acid, as hereinbefore described (e.g., an acid suitable for step (B)), in a suitable solvent;
1.83 Method 1.82, wherein the oxidizing agent is selected from one or more of hydrogen peroxide, a chromium oxidant (e.g., chromium trioxide, chromic acid, pyridinium chlorochromate, potassium dichromate, chromium trioxide-pyridine complex, pyridinium dichromate), osmium tetroxide, potassium permanganate, peracetic acid, perchloric acid, perbenzoic acid, meta-chloroperoxybenzoic acid (mCPBA), trifluoroperacetic acid, periodic acid, magnesium monoperoxyphthalate, dimethyl dioxirane (DMDO), tert-butyl hydroperoxide, sodium hypochlorite, sodium tungstate, sodium periodate, potassium periodate, iodosyl benzene, pentafluoroiodosyl benzene, cumene hydroperoxide, potassium persulfate, potassium monoperoxysulfate, pyridine N-oxide, 2,6-dichloropyridine N-oxide, sodium chlorite, sodium hypochlorite, sodium chlorate, sodium perchlorate, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries);
1.84 Method 1.83, wherein the oxidizing agent is hydrogen peroxide, peracetic acid, trifluoroperacetic acid, meta-chloroperoxybenzoic acid, tert-butyl hydroperoxide, or potassium monoperoxysulfate;
1.85 Any of Methods 1.82-1.84, wherein the reaction is carried out using 1.0 to 5.0 equivalents of the oxidizing agent, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents; and optionally 0.01 to 1.0 equivalents of any one or more additional reagents (e.g., secondary oxidant, or catalyst, or ligand, or other agent), e.g., 0.01 to 0.5 equivalents, 0.01 to 0.2 equivalents, or 0.01 to 0.1, or 0.01 to 0.05 equivalents;
1.86 Any of Methods 1.82-1,84, wherein the acid is a Bronsted acid, e.g., selected from hydrochloric acid, perchloric acid, sulfuric acid, phosphoric acid, acetic acid, trifluoromethanesulfonic acid, methanesulfonic acid, trifluoroacetic acid, and nitric acid, or a heteropoly acid (e.g., phosphotungstic acid);
1.87 Method 1.86, wherein the acid is selected from sulfuric acid, phosphoric acid, and nitric acid;
1.88 Any of Methods 1.82-1.87, wherein the reaction is carried out using 1.0 to 5.0 equivalents of the acid, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents;
1.89 Any of Methods 1.82-1.88, wherein the reaction is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
1.90 Method 1.89, wherein the solvent is acetic acid and/or acetic anhydride and optionally wherein the reaction further comprises an acetate salt (e.g., sodium or potassium acetate);
1.91 Any of Methods 1.81-1.90, wherein the reaction is carried out using a combination of hydrogen peroxide, sulfuric acid, and acetic acid, optionally 1.5-4 equivalents of each (e.g., 2-3 equivalents of each);
1.92 Any of Methods 1.81-1.90, wherein the reaction is carried out using a combination of acetic acid, peracetic acid, and sulfuric acid, optionally 1-3 equivalents of each (e.g., 1-1.5 equivalents of each);
1.93 Any of Methods 1.81-1.90, wherein the reaction is carried out using a combination of hydrogen peroxide, acetic acid, peracetic acid, and sulfuric acid, optionally 0.8-5 equivalents of each (e.g., 1-3 equivalents of each), optionally with sodium acetate (e.g., 0.05-0.24 equivalents);
1.94 Any of Methods 1.81-1.90, wherein the reaction is carried out using a combination of hydrogen peroxide, acetic anhydride, acetic acid, and sulfuric acid, optionally 0.8-5 equivalents of each (e.g., 1-3 equivalents of each), optionally with sodium acetate (e.g., 0.05-0.24 equivalents), further optionally with heptane co-solvent;
1.95 Any of Methods 1.81-1.90, wherein the reaction is carried out using a combination of potassium monoperoxysulfate, potassium hydrogen sulfate and potassium sulfate (e.g., Oxone®), in organic solvent (e.g., acetone/ethyl acetate), optionally 1-3 equivalents of potassium monoperoxysulfate (e.g., 1-2 equivalents);
1.96 Any of Methods 1.81-1.95, wherein the reaction is carried out at a temperature of 0° C. to 200° C., e.g., 0° C. to 150° C., or 0° C. to 100° C., or 25° C. to 200° C., or 25° C. to 150° C., or 25° C. to 100° C., or 50° C. to 200° C., or 50° C. to 150° C., or 50° C. to 100° C., or 75° C. to 200° C., or 75° C. to 150° C., or 75° C. to 100° C., or 80° C. to 150° C., or 80° C. to 100° C., or 90° C. to 150° C., or 90° C. to 125° C., or 90° C. to 110° C., or 90° C. to 100° C., or 90° C. to 95° C.;
1.97 Any of Methods 1.81-1.96, wherein the reaction is carried out at a temperature of 80° C. to 90° C., 20° C. to 70° C., 40° C. to 90° C., 20° C. to 50° C., or 20° C. to 30° C.;
1.98 Any of Methods 1.81-1.97, wherein the reactions steps (A), (B), (C), and/or (D) are carried out in a single vessel in a single reaction step, e.g., wherein step (B) spontaneously follows step (A), and step (C) spontaneously follow step (B), and step (D) spontaneously follows step (C);
1.99 Any of Methods 1.81-1.97, wherein the reactions steps (A), (B), (C), and/or (D) are carried out in a single vessel in a two reaction steps without isolation of any intermediates, e.g., wherein at least a second oxidizing agent is added in the second reaction step to initiate step (C), and wherein step (B) spontaneously follows step (A) and step (D) spontaneously follows step (C);
1.100 Any of Methods 1.81-1.99, wherein R is H, and wherein the reactant Compound (2) proceeds to the product Compound (1) in a single vessel, wherein the reaction is carried out by treating the Compound (2) with a combination of hydrogen peroxide, sulfuric acid, and acetic acid, optionally 1.5-4 equivalents of each (e.g., 2-3 equivalents of each, or 2 equivalents of each), optionally at a temperature of 75° C. to 150° C., or 80° C. to 100° C., or 90° C. to 95° C.;
1.101 Method 1, or any of 1.1-1.100, wherein the method further comprises a Step (E) of treating the Compound (1) with a base to cause enrichment of the Compound (1a) by isomerization of the Compound (1b) to the Compound (1a);
1.102 Method 1.101, wherein the base is selected from sodium hydride, potassium hydride, hydroxide bases (e.g., sodium hydroxide, potassium hydroxide), alkoxide bases (e.g., sodium tert-butoxide, potassium tert-butoxide, sodium methoxide, potassium methoxide, sodium ethoxide, potassium ethoxide), carbonate bases (e.g., sodium carbonate, potassium carbonate, cesium carbonate), bicarbonate bases (e.g., sodium bicarbonate), amide bases (e.g., lithium amide, lithium 2,2,6,6-tetramethylpiperide (LiTMP), lithium diisopropylamide (LDA), lithium bis(trimethylsilyl)amide) (LiHMDS)), alkyl lithium bases (e.g., sec-butyl lithium, tert-butyl lithium), and amine bases (e.g., triethylamine, diisopropylethylamine, N-methyl morpholine, DBU, DBN, pyridine, dimethylaminopyridine, imidazole);
1.103 Method 1.102, wherein the base is an alkoxide base (e.g., sodium tert-butoxide, potassium tert-butoxide, sodium methoxide, potassium methoxide, sodium ethoxide, potassium ethoxide) or an amide base (e.g., lithium amide, lithium 2,2,6,6-tetramethylpiperide (LiTMP), lithium diisopropylamide (LDA), lithium bis(trimethylsilyl)amide) (LiHMDS));
1.104 Method 1.102, wherein the base is sodium tert-butoxide or potassium tert-butoxide;
1.105 Any of Methods 1.101-1.104, wherein step (E) is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), and ethers (e.g., diethyl ether, diisopropyl ether, methyl tert-butyl ether, tetrahydrofuran, 1,4-dioxane, dimethoxyethane, diethylene glycol dimethyl ether);
1.106 Method 1.105, wherein step (E) is carried out in a hydrocarbon solvent, e.g., pentane, hexane, heptane, or cyclohexane.
1.107 Any of Methods 1.101-1.106, wherein step (E) is carried out at a temperature of −78° C. to 200° C., e.g., −50° C. to 150° C., or −25° C. to 100° C., or 0 to 75° C., or 15° C. to 50° C., or 20° C. to 30° C., −78° C. to 0° C., or −50° C. to 0° C., or −25° C. to 25° C., or 25° C. to 75° C., or 50° C. to 100° C., or 75° C. to 150° C., or 100° C. to 150° C., or 125° C. to 200° C., or 150° C. to 200° C.;
1.108 Any of Methods 1.101-1.107, wherein Step (E) provides an enrichment in the amount of the isomer (1a) of at least 10% by weight of the total weight of Compound (1), e.g., at least 15%, or at least 20%, or at least 25%;
1.109 Any of Methods 1.101-1.108, wherein Step (E) provides a product having a ratio of isomer (1a) to isomer (1b) of at least 90:10, e.g., at least 92:8, or at least 93:7, or at least 94:6, or at least 95:5;
1.110 Method 1, or any of 1.1-1.109, wherein the Method further comprises the crystallization of the product Compound (1) from a hydrocarbon solvent (e.g., pentane, hexane, heptane, cyclohexane, or a mixture thereof) at a temperature of less than 30° C., e.g., less than 25° C., or less than 15° C., or less than 10° C., or less than 5° C., or less than 0° C. to, or less than ˜5° C., or less than ˜10° C., or less than ˜20° C., or less than ˜30° C., or less than ˜40° C., or less than ˜50° C., e.g., as low as −78° C.;
1.111 Method 1.110, wherein the recrystallization step provides a product having a ratio of isomer (1a) to isomer (1b) of at least 95:5, e.g., at least 97:3, or at least 98:2, or at least 99:1, or at least 99.5:0.5;
1.112 Any of Methods 1.101-1.111, wherein the Method comprises isomerization step (E) followed by recrystallization of the product (1);
1.113 Method 1.112, wherein the isomerization step (E) and the recrystallization step are performed as a continuous process in which each recrystallization is followed by an isomerization followed by a recrystallization, etc., until a desired purity of the isomer (1a) is obtained;
1.114 Method 1.113, wherein the continuous isomerization and recrystallization steps provide a product having a ratio of isomer (1a) to isomer (1b) of at least 95:5, e.g., at least 97:3, or at least 98:2, or at least 99:1, or at least 99.5:0.5
1.115 Method 1, or any of 1.1-1.114, wherein the method does not comprise the use of any reagents or reactants other than the Compound (2), and the reagents set forth herein (e.g., acids, bases, oxidizing agents, catalysts, protecting agents, deprotecting agents, solvents), for example, the method does not comprise the use of any carbon monoxide, carbonyl equivalents, or enzymes).

In further embodiments of Method 1, the present disclosure provides Method 1, or any of 1.1-1.115, wherein step (B) provides as a by-product, one or more of the compounds of Formula XIII, XIV and XV:

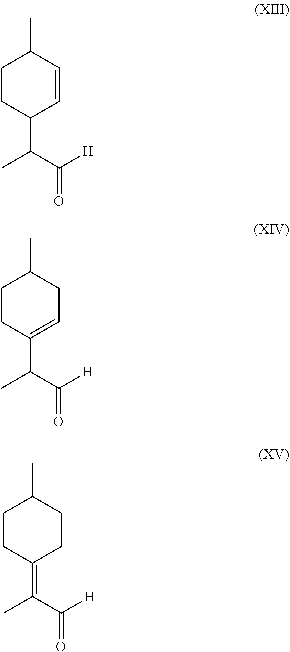

In particular embodiments, the present disclosure provides Method 1, or any of 1.1-1.115, wherein step (B) provides as a by-product, one or more of the compounds of Formula XIIIa, XIIIb, XIVa, XIVb, and XVa:

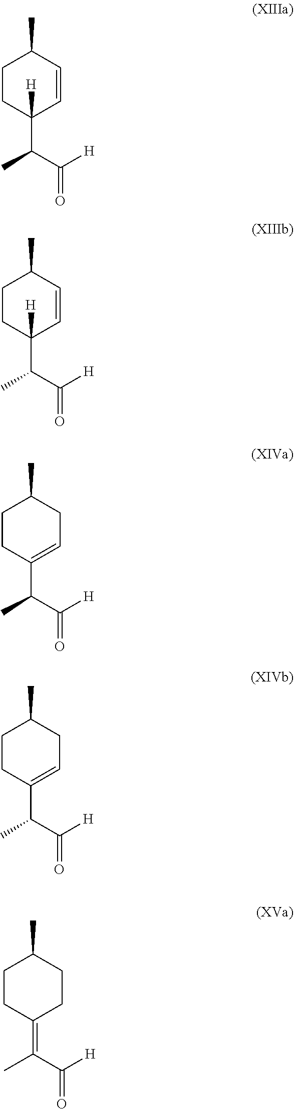

Without being bound by theory, it is believed that the double bond of the compounds according to Formulas XIII, XIV, XV, XIIIa, XIIIb, XIVa, XIVb, and/or XVa, may form during step (A) and/or step (B) of Method 1 et seq., such as, by way of acid-catalyzed, base-catalyzed, or thermal elimination of the hydroxy group (—OH) or protected hydroxy group (—OR) of one or more of the compounds of Formula (2), (3), (3a), (3b), (4), (4a), or (4b). This may be particularly likely when the group R is —S(O)₂—R¹, for example, wherein R¹ is methyl, trifluoromethyl, phenyl, or tolyl. Thus, for example, the following reactions may occur during steps (A) and (B):

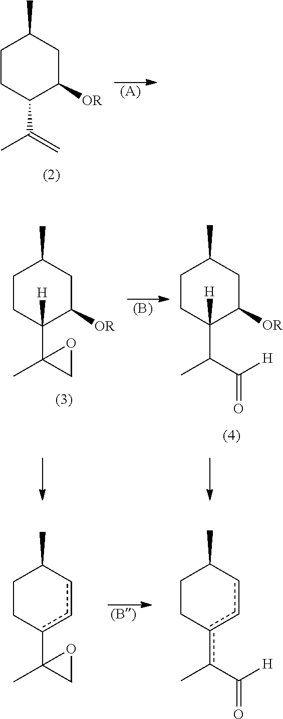

As shown above, under the conditions of step (A), the initially formed compound (3) may undergo elimination of OR to form the unsaturated epoxide compound (step A′ above), followed by, under the conditions of step (B), the conversion of the unsaturated epoxide compound to the unsaturated aldehyde (step B″ above). Alternatively, both of these conversions may occur under the conditions of step B (step B′ followed by step B″ above). Alternatively, under the conditions of step (B), the compound (4) may undergo elimination of OR to form the unsaturated epoxide compound (step B′″ above).

Optionally, the compound of Formulas XIII, XIV, XV, XIIIa, XIIIb, XIVa, XIVb, and/or XVa, may be isolated (e.g., extracted, distilled, or separated via chromatography) from the product mixture of step (B).

In a second aspect, the present disclosure provides, a method (Method 2) of making an optionally 3,6-disubstituted hexahydrobenzofuran-2-one (Compound 6) comprising the steps of:
(A) epoxidizing an optionally 5-substituted-2-vinylcyclohexanol, or a derivative thereof (Compound 7), to form epoxide Compound (8);
(B) rearranging epoxide Compound (8) to form aldehyde Compound (9);
(C) oxidizing aldehyde Compound (9) to form carboxylic acid Compound (10); and
(D) ring closing the carboxylic acid Compound (10) to form the Compound (6):

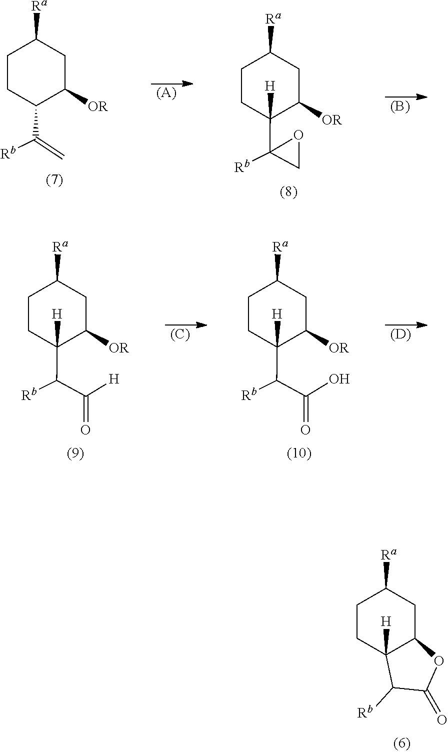

wherein R is H or a protecting group (e.g., an ether, an ester, or a silyl ether protecting group), and R^{a} is selected from H, optionally substituted C₁₋₆alkyl, optionally substituted C₂₋₆alkenyl, optionally substituted C₂₋₆alkynyl, optionally substituted aryl, O—R^{c}, and —C(O)—R^{c}, and R^{b} is selected from H, optionally substituted C₁₋₆alkyl, optionally substituted aryl, O—R^{c}, and —C(O)—R^{c}, and R^{c} is selected from H, optionally substituted C₁₋₆alkyl, optionally substituted C₂₋₆alkenyl, optionally substituted C₂₋₆alkynyl, and optionally substituted aryl.

In further embodiments of the first aspect, the present disclosure provides:
2.1 Method 2, wherein R is H;
2.2 Method 2, wherein R is an ester protecting group, e.g., —C(O)—R¹ or —S(O)₂—R¹, and wherein R¹ is H, C₁₋₆alkyl (e.g., methyl or ethyl), haloC₁₋₆alkyl (e.g., chloromethyl or trifluoromethyl), C₁₋₆alkoxy (e.g., methoxy or ethoxy), C₁₋₆alkoxymethyl (e.g., methoxyethyl or ethoxymethyl), aryl (e.g., phenyl or tolyl), arylmethyl (e.g., benzyl), aryloxy (e.g., phenoxy), or aryloxymethyl (e.g., phenoxymethyl);
2.3 Method 2.2, wherein R is —C(O)—R¹, and wherein R¹ is methyl, ethyl, propyl, isopropyl, or tert-butyl, or wherein R is —S(O)₂—R¹, and wherein R¹ is methyl;
2.4 Method 2.2, wherein R is —C(O)—R¹, and wherein R¹ is methyl;
2.5 Method 2.2, wherein R is —C(O)—R¹, and wherein R¹ is chloromethyl, dichloromethyl, trichloromethyl, 2,2,2-trichloroethyl, trifluoromethyl, methoxymethyl, phenoxymethyl, or benzyl, or wherein R is —S(O)₂—R¹, and wherein R¹ is trifluoromethyl, phenyl or tolyl;
2.6 Method 2, wherein R is an ether protecting group, e.g., R is unsubstituted C₁₋₆alkyl (e.g., methyl, ethyl, isopropyl, tert-butyl), or substituted C₁₋₆alkyl, such as C₁₋₆alkoxy-C₁₋₆alkyl, aryloxy-C₁₋₆alkyl, or aryl-C₁₋₆alkyl (e.g., —CH₂—O-Me, —CH₂—O-Et, —CH₂—S-Me, —CH₂—O—CH₂CH₂—OMe, CH₂—O—CH₂CCl₃, CH₂—O—CH₂CH₂—SiMe₃, —CH₂—O-Ph, —CH₂—O—CH₂-Ph, —CH₂—O—CH₂-(4-methoxyphenyl), —CH₂—O—CH₂-(3,4-dimethoxyphenyl), —CH₂CH₂-OEt, —CH₂CH₂Si(Me)₃, —CH₂CCl₃, —CH₂-Ph, —CH₂-(4-methoxyphenyl), —CH₂-(3,4-dimethoxyphenyl), —CH₂-(2,6-dimethoxyphenyl), or tetrahydropyranyl;
2.7 Method 2, wherein R is a silyl ether protecting group (e.g., R is —Si(R²)(R³)(R⁴), wherein R², R³, and R⁴ are each independently selected from C₁₋₆alkyl (e.g., methyl, ethyl, isopropyl, tert-butyl, thexyl, benzyl), C₁₋₆alkoxy (e.g., methoxy, ethoxy, tert-butoxy), and aryl (e.g., phenyl);
2.8 Method 2.7, wherein R is selected from trimethylsilyl, triethylsilyl, triisopropylsilyl, dimethylisopropylsilyl, diethylisopropylsilyl, dimethylthexylsilyl, tert-butyldimethylsilyl, tert-butyldiphenylsilyl, tribenzylsilyl, triphenylsilyl, diphenylmethylsilyl, di-tert-butylmethylsilyl, tris(trimethylsilyl)silyl, tert-butylmethoxyphenylsilyl, and tert-butoxydiphenylsilyl;
2.9 Method 2 or any of 2.1 to 2.8, wherein the Compound (8) is Compound (8a), Compound (8b) or a mixture thereof:

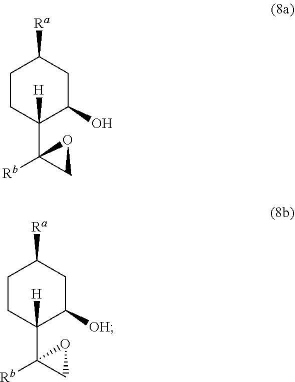

2.10 Method 2 or any of 2.1 to 2.9, wherein the Compound (9) is Compound (9a), Compound (9b) or a mixture thereof:

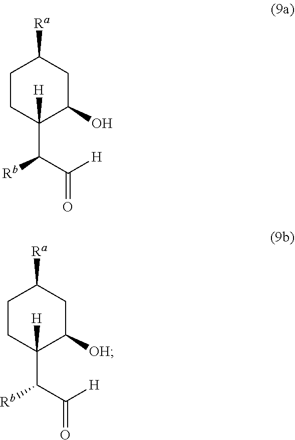

2.11 Method 2 or any of 2.1 to 2.10, wherein the Compound (10) is Compound (10a), Compound (10b) or a mixture thereof:

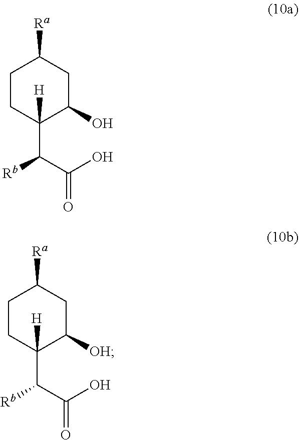

2.12 Method 2 or any of 2.1 to 2.11, wherein the Compound (6) is Compound (6a), Compound (6b) or a mixture thereof:

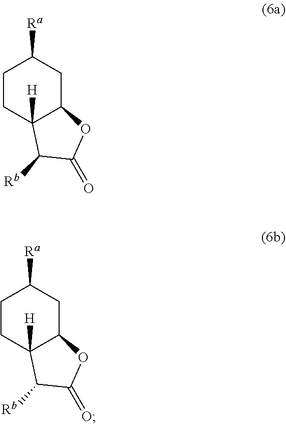

optionally, wherein the Compound (6) is enriched in one isomer or the other isomer, or wherein the method comprises the step of purifying or separating the isomers;
2.13 Method 2, or any of 2.1-2.12, further comprising the step (A′) of converting Compound (7′), wherein R is H, to Compound (7″), wherein R is not H (e.g., wherein R is a protecting group):

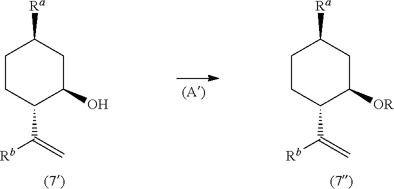

2.14 Method 2.13, wherein step (A′) is the first step in method, immediately preceding step (A);
2.15 Method 2, or any of 2.1-1.14, further comprising the step (D′) of converting Compound (10′), wherein R is not H (e.g., wherein R is a protecting group), to Compound (10″), wherein R is H:

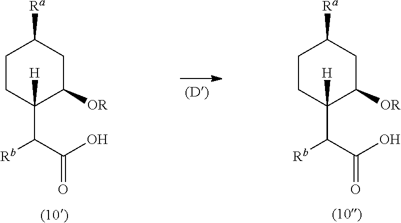

2.16 Method 2.15, wherein step (D′) is the penultimate step in method, immediately preceding step (D);
2.17 Method 2, or any of 2.1-2.16, wherein the protecting group R is eliminated during step (D) (i.e., no deprotection step D′ is needed);
2.18 Method 2, or any of 2.1-2.17, wherein the method does not comprise any step using a compound VI′ as an intermediate

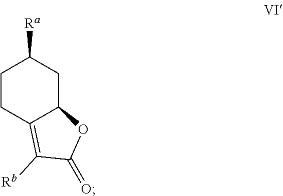

2.19 Method 2, or any of 2.1-2.18, wherein the method does not comprise any step using compound (IV′), compound (V′), or compound (VII′), as an intermediate

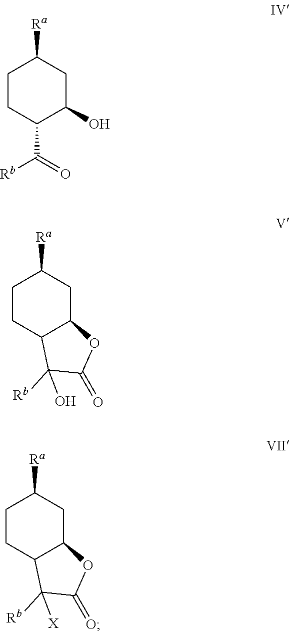

wherein X is Cl, Br, or I;
2.20 Method 2, or any of 2.1-2.19, wherein the method does yield any measurable amount of compound (XI) or compound (XII), as described hereinabove, e.g., as measured by HPLC, GC, MS, or NMR;
2.21 Method 2, or any of 2.1-2.20, wherein the method does not comprise any synthetic steps and/or mechanistic steps other than steps (A), (B), (C), and/or (D), and optionally (A′) and/or (D′) from Compound (7) or Compound (7′) through Compound (6);
2.22 Method 2, or any of 2.1-2.21, wherein epoxidation step (A) is carried out by treating the compound (6) with a suitable oxidizing agent in a suitable solvent;
2.23 Method 2.22, wherein the suitable oxidizing agent is one or more of hydrogen peroxide, osmium tetroxide, peracetic acid, perchloric acid, perbenzoic acid, meta-chloroperoxybenzoic acid (mCPB A), trifluoroperacetic acid, magnesium monoperoxyphthalate, dimethyl dioxirane (DMDO), tert-butyl hydroperoxide, sodium hypochlorite, sodium tungstate, sodium periodate, iodosyl benzene, pentafluoroiodosyl benzene, cumene hydroperoxide, potassium persulfate, potassium monoperoxysulfate, pyridine N-oxide, 2,6-dichloropyridine N-oxide, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries);
2.24 Method 2.22, wherein in epoxidation step (A) the suitable oxidizing agent is hydrogen peroxide, peracetic acid, meta-chloroperoxybenzoic acid, tert-butyl hydroperoxide, or potassium monoperoxysulfate;
2.25 Method 2.22, wherein in epoxidation step (A) the suitable oxidizing agent is hydrogen peroxide and sodium tungstate, e.g., hydrogen peroxide (e.g., 30 wt. %, e.g., 1-1.5 equivalents) plus sodium tungstate (e.g., sodium tungstate dihydrate, e.g., 0.01-0.10 equivalents) with methyl-tri-n-octylammonium hydrogen sulfate (e.g., 0.01-0.05 equivalents) and phenylphosphonic acid (e.g., 0.01-0.05 equivalents), optionally at 0-50° C., optionally in aqueous solvent;
2.26 Method 2.22, wherein in epoxidation step (A) the suitable oxidizing agent is m-chloroperoxybenzoic acid;
2.27 Method 2.22, wherein in epoxidation step (A) the suitable oxidizing agent is oxygen gas;
2.28 Method 2.27, wherein in epoxidation step (A) the suitable oxidizing agent is oxygen gas with a transition metal catalyst;
2.29 Method 2.27, wherein in epoxidation step (A) the suitable oxidizing agent is oxygen gas and iron(III)-tetraphenylporphyrin complex (Fe(III)TPP), in the presence of a C₂₋₁₀ aliphatic aldehyde (e.g., isobutyraldehyde);
2.30 Method 2.27, wherein in epoxidation step (A) the suitable oxidizing agent is oxygen gas and N-hydroxyphthalimide;
2.31 Any of Methods 2.22-2.30, wherein in epoxidation step (A) does not comprise the use of ozone;
2.32 Any of Methods 2.22-2.31, wherein in epoxidation step (A) the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
2.33 Any of Methods 2.22-2.32, wherein the epoxidation step (A) is carried out using 1.0 to 5.0 equivalents of the oxidizing agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents; and optionally 0.01 to 1.0 equivalents of any one or more additional reagents (e.g., secondary oxidant, or catalyst, or ligand, or other agent), e.g., 0.01 to 0.5 equivalents, 0.01 to 0.2 equivalents, or 0.01 to 0.1, or 0.01 to 0.05 equivalents;
2.34 Any of Methods 2.22-2.33, wherein the epoxidation step (A) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
2.35 Any of Methods 2.22-2.34, wherein the epoxidation step (A) is carried out in a batch reactor;
2.36 Any of Methods 2.22-2.34, wherein the epoxidation step (A) is carried out in a continuous flow reactor;
2.37 Method 2, or any of 2.1-2.36, wherein the rearrangement step (B) is carried out by treating the compound (7) with a suitable rearrangement catalyst in a suitable solvent, or by heating the compound (3) without catalyst in a suitable solvent (i.e., thermal rearrangement);
2.38 Method 2.37, wherein the rearrangement catalyst is a Lewis acid, a Bronsted acid, a strong base (e.g., LDA, LiTMP, LiHMDS, t-butyl lithium), or a transition metal catalyst or complex (e.g., palladium, ruthenium, rhodium, chromium, iridium, zirconium, manganese, iron, or nickel catalyst or complex);
2.39 Method 2.37, wherein the rearrangement catalyst is a solid phase acidic resin (e.g., an acidic polymer resin such as Amberlyst or Nafion-H, or a Montmorillonite, or a Zeolite), e.g., Montmorillonite K10, or Amberlyst H-15, optionally wherein the catalyst is Montmorillonite K-10, e.g., at 0.1-0.5 equivalents, e.g., in toluene solvent, e.g., at 0-50° C. (e.g., about 25° C.);
2.40 Method 2.37, wherein the rearrangement catalyst is a Lewis acid, e.g., selected from zinc bromide, zinc chloride, magnesium bromide, magnesium bromide-diethyl ether complex, bismuth triflate, boron trifluoride-diethyl ether complex, aluminum triisopropoxide, titanium tetraisopropoxide, titanium tetrachloride, iron trichloride, indium chloride, lithium perchlorate, iridium chloride, lithium bromide, borane-THF complex, chromium tetraphenyl porphyrin triflate, nickel bis(triphenylphosphine) dibromide complex, methyl bis(4-bromo-1,6-di-tert-butylphenoxy) aluminum;
2.41 Method 2.37, wherein the rearrangement catalyst is a Bronsted acid, e.g., selected from hydrochloric acid, perchloric acid, sulfuric acid, phosphoric acid, acetic acid, peracetic acid, trifluoromethanesulfonic acid, methanesulfonic acid, trifluoroacetic acid, and nitric acid, or a heteropoly acid (e.g., phosphotungstic acid);
2.42 Any of Methods 2.37-2.41, wherein in rearrangement step (B) the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
2.43 Any of Methods 2.37-2.42, wherein rearrangement step (B) is carried out using
1.0 to 5.0 equivalents of the rearrangement catalyst, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents; or using 0.01 to 1.0 equivalents of the rearrangement catalyst, e.g., 0.01 to 0.1 equivalents, 0.1 to 0.5 equivalents, or 0.5 to
1.0 equivalents;
2.44 Any of Methods 2.37-2.43, wherein rearrangement step (B) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
2.45 Any of Methods 2.37-2.44, wherein rearrangement step (B) is carried out in a batch reactor;
2.46 Any of Methods 2.37-2.45, wherein rearrangement step (B) is carried out in a continuous flow reactor;
2.47 Method 2, or any of 2.1-2.46, wherein rearrangement step (B) is carried out in the same vessel as oxidation step (C), e.g., wherein the product of step (B) is not purified or isolated before carrying out step (C), or wherein the reagent or reagents and solvent for step (C) is added directly to the reaction mixture of step (B);
2.48 Method 2, or any of 2.1-2.47, wherein oxidation step (C) is carried out by treating the compound (8) with a suitable oxidizing agent in a suitable solvent;
2.49 Method 2.48, wherein in oxidation step (C) the suitable oxidizing agent is one or more of a chromium oxidant (e.g., chromium trioxide, chromic acid, pyridinium chlorochromate, potassium dichromate, chromium trioxide-pyridine complex, pyridinium dichromate), osmium tetroxide, potassium permanganate, silver oxide, hydrogen peroxide, peracetic acid, perchloric acid, trifluoroperacetic acid, periodic acid, potassium periodate, sodium chlorite, oxygen and N-hydroxyphthalimide, potassium persulfate, and potassium monoperoxysulfate;
2.50 Method 2.48, wherein in oxidation step (C) the suitable oxidizing agent is a chromium oxidant (e.g., chromium trioxide, chromic acid, pyridinium chlorochromate, potassium dichromate, chromium trioxide-pyridine complex, pyridinium dichromate), optionally wherein the chromium oxidant is chromium trioxide in aqueous sulfuric acid (i.e., Jones Reagent) (e.g., about 1-1.5 equivalents Jones Reagent, e.g., in acetone solvent, e.g., at 0-50° C. (e.g., about 25° C.);
2.51 Any of Methods 2.48-2.50, wherein in oxidation step (C) does not comprise the use of ozone;
2.52 Any of Methods 2.48-2.51, wherein in oxidation step (C) the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
2.53 Any of Methods 2.48-2.52, wherein the oxidation step (C) is carried out using 1.0 to 5.0 equivalents of the oxidizing agent, 1.0 to 5.0 equivalents of the rearrangement catalyst, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents;
2.54 Any of Methods 2.48-2.53, wherein the oxidation step (C) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
2.55 Any of Methods 2.48-2.54, wherein the oxidation step (C) is carried out in a batch reactor;
2.56 Any of Methods 2.48-2.54, wherein the oxidation step (C) is carried out in a continuous flow reactor;
2.57 Method 2, or any of 2.1-2.56, wherein ring closure step (D) and/or deprotection of the group —OR of the compound (10) occurs spontaneously during and/or after oxidation step (C);
2.58 Method 2, or any of 2.1-2.56, wherein ring closure step (D) and/or deprotection of the group —OR of the compound (10) is carried out by heating the product mixture from step (C);
2.59 Method 2, or any of 2.1-2.56, wherein ring closure step (D) and/or deprotection of the group —OR of the compound (10) is carried out by distilling the product mixture from step (C);
2.60 Method 2, or any of 2.1-2.56, wherein the method comprises deprotection step (D′) immediately prior to ring closure step (D) and immediately after oxidation step (C);
2.61 Method 2.60, wherein the deprotection step (D′) is carried out by treating the compound (5) with aqueous base, aqueous acid, anhydrous base, anhydrous acid, or anhydrous fluoride, or biphasic acid, biphasic base, or biphasic fluoride, or other suitable conditions (e.g., sodium azide, mercuric chloride, magnesium bromide, magnesium iodide, DDQ (2,3-dichloro-5,6-dicyano-1,4-benzoquinone, ceric ammonium nitrate, hydrogen over palladium or platinum catalyst), in a suitable solvent;
2.62 Method 2.61, wherein the base is selected from lithium hydroxide, sodium hydroxide, potassium hydroxide, sodium methoxide, potassium methoxide, potassium tert-butoxide, sodium carbonate, potassium carbonate, cesium carbonate, ammonium hydroxide, tetrabutylammonium hydroxide, ammonia, guanidine, ethylene diamine, ethanolamine, pyridine, lutidine, collidine, triethylamine, diisopropylethylamine, piperidine, morpholine, methylamine, hydrazine, and imidazole;
2.63 Method 2.62, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or combinations thereof (including aqueous or anhydrous or biphasic combinations thereof);
2.64 Method 2.61, wherein the acid is selected from hydrochloric acid (e.g., hydrogen chloride), nitric acid, sulfuric acid, phosphoric acid, acetic acid, peracetic acid, formic acid, citric acid, trifluoroacetic acid, perchloric acid, trifluoromethanesulfonic acid, methanesulfonic acid, hydrogen fluoride, hydrogen bromide, boron trifluoride-etherate, toluenesulfonic acid, scandium triflate, ytterbium triflate, pyridinium para-toluenesulfonate (PPTS), zinc bromide, zinc chloride, titanium chloride, tin(IV) chloride, bromodimethylborane, boron trichloride, pyridine-HF complex, and solid acidic resins (e.g., acidic polymers such as Amberlyst H-15; silica gel, alumina, Montmorillonite K-10);
2.65 Method 2.64, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or combinations thereof (including aqueous or anhydrous or biphasic combinations thereof);
2.66 Method 2.61, wherein the fluoride is selected from hydrogen fluoride (e.g., anhydrous HF, aqueous HF, triethylamine HF complex, pyridine HF complex), TASF (tris(dimethylamino)sulfonium difluorotrimethylsilicate), sodium fluoride, potassium fluoride, cesium fluoride, ammonium fluoride, and tetrabutylammonium fluoride;
2.67 Method 2.66, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid), carbon dioxide (e.g., supercritical carbon dioxide), and carbon disulfide, or combinations thereof (including aqueous or anhydrous or biphasic combinations thereof);
2.68 Any of Methods 2.60-2.67, wherein the deprotection step (D′) is carried out using
1.0 to 5.0 equivalents of the acid, base, fluoride, or other agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents;
2.69 Any of Methods 2.60-2.68, wherein the deprotection step (D′) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
2.70 Any of Methods 2.60-2.69, wherein the deprotection step (D′) is carried out in a batch reactor;
2.71 Any of Methods 2.60-2.70, wherein the deprotection step (D′) is carried out in a continuous flow reactor;
2.72 Method 2, or any of 2.1-2.71, further comprising the protection step (A′);
2.73 Method 2.72, wherein protection step (A′) is carried out by treating the compound (7) with a suitable protecting agent in a suitable solvent, optionally with a suitable base;
2.74 Method 2.73, wherein the protecting agent is selected from an acyl halide (e.g., acetyl chloride, benzoyl chloride, chloroacetyl chloride, dichloroacetyl chloride, trichloroacetyl chloride, methoxyacetyl chloride, phenoxyacetyl chloride, pivaloyl chloride, benzoyl chloride), an acyl anhydride (e.g., acetic anhydride, chloroacetic anhydride, dichloroacetic anhydride, trichloroacetic anhydride, trifluoroacetic anhydride, methoxyacetyl anhydride, phenoxyacetyl anhydride, pivaloyl anhydride, benzoyl anhydride), an alkyl halide (e.g., methoxymethyl chloride, methoxymethyl bromide, methoxyethyl chloride, methylthiomethyl iodide, benzyloxymethyl chloride, 4-methoxybenzyloxymethyl chloride, 2-methoxyethoxymethyl chloride, 2,2,2-trichloroethoxymethyl chloride, 2-trimethylsilylethoxymethyl chloride, 4-methoxybenzyl chloride, 4-methoxybenzyl bromide, 3,4-dimethoxybenzyl bromide,), a silyl reagent (e.g., the chloride, silane or triflate of the group trimethylsilyl, triethylsilyl, triisopropylsilyl, dimethylisopropylsilyl, diethylisopropylsilyl, dimethylthexylsilyl, tert-butyldimethylsilyl, tert-butyldiphenylsilyl, tribenzylsilyl, triphenylsilyl, diphenylmethylsilyl, di-tert-butylmethylsilyl, tris(trimethylsilyl)silyl, tert-butylmethoxyphenylsilyl, and tert-butoxydiphenylsilyl), or other protecting agents (e.g., formic acid, acetic acid, ethyl formate, methyl formate, chloroacetic acid, dihydropyran, 2-hydroxytetrahydropyran, ethyl vinyl ether, trimethylsilylethoxyethene, isobutylene, methanesulfonyl chloride, trifluoromethanesulfonyl chloride, trifluoromethanesulfonic anhydride, N,N-bis(trifluoromethanesulfonyl)aniline, benzenesulfonyl chloride, toluenesulfonyl chloride);
2.75 Method 2.73 or 2.74, wherein the suitable base is selected from sodium hydride, potassium hydride, hydroxide bases (e.g., sodium hydroxide, potassium hydroxide), alkoxide bases (e.g., sodium tert-butoxide, potassium tert-butoxide), carbonate bases (e.g., sodium carbonate, potassium carbonate, cesium carbonate), bicarbonate bases (e.g., sodium bicarbonate), and amine bases (e.g., triethylamine, diisopropylethylamine, N-methyl morpholine, DBU, DBN, pyridine, dimethylaminopyridine, imidazole);
2.76 Any of methods 2.73-2.75, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid), carbon dioxide (e.g., supercritical carbon dioxide), and carbon disulfide, or combinations thereof, or wherein the solvent is a neat reagent (e.g., pyridine);
2.77 Any of Methods 2.73-2.76, wherein the protection step (A′) is carried out using
1.0 to 5.0 equivalents of the protecting agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents;
2.78 Any of Methods 2.73-2.77, wherein the protection step (A′) is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
2.79 Any of Methods 2.73-2.78, wherein the protection step (A′) is carried out in a batch reactor;
2.80 Any of Methods 2.73-2.79, wherein the protection step (A′) is carried out in a continuous flow reactor;
2.81 Method 2, or any of 2.1-2.80, wherein R^{a} is H;
2.82 Method 2, or any of 2.1-2.80, wherein R^{a} is optionally substituted C₁₋₆alkyl;
2.83 Method 2, or any of 2.1-2.80, wherein R^{a} is optionally substituted aryl;
2.84 Method 2, or any of 2.1-2.80, wherein R^{a} is O—R^{c};
2.85 Method 2, or any of 2.1-2.80, wherein R^{a} is —C(O)—R^{c};
2.86 Method 2.84 or 2.85, wherein R^{c} is selected from H, optionally substituted C₁₋₆alkyl, and optionally substituted aryl;
2.87 Method 2.84 or 2.85, wherein R^{c} is selected from H and optionally substituted C₁₋₆ alkyl;
2.88 Method 2, or any of 2.1-2.87, wherein R^{b} is H;
2.89 Method 2, or any of 2.1-2.87, wherein R^{b} is optionally substituted C₁₋₆alkyl;
2.90 Method 2, or any of 2.1-2.87, wherein R^{b} is optionally substituted aryl;
2.91 Method 2, or any of 2.1-2.87, wherein R^{b} is selected from 0-R^{c} and —C(O)—R^{c};
2.92 Method 2.91, wherein R^{c} is selected from H, optionally substituted C₁₋₆alkyl, and optionally substituted aryl;
2.93 Method 2.91, wherein R^{c} is selected from H and optionally substituted C₁₋₆alkyl;
2.94 Any of Methods 2.1-2.93, wherein the intermediates (8), (9), and (10), of steps (A), (B), (C), and (D) are not isolated, e.g., wherein the reactant Compound (7) proceeds to the product Compound (6) in a single vessel;
2.95 Method 2.94, wherein the reaction comprises the treatment of Compound (7) with an oxidizing agent, as hereinbefore described (e.g., an oxidizing agent suitable for step (A) or for step (C)), and an acid, as hereinbefore described (e.g., an acid suitable for step (B)), in a suitable solvent;
2.96 Method 2.95, wherein the oxidizing agent is selected from one or more of hydrogen peroxide, a chromium oxidant (e.g., chromium trioxide, chromic acid, pyridinium chlorochromate, potassium dichromate, chromium trioxide-pyridine complex, pyridinium dichromate), osmium tetroxide, potassium permanganate, peracetic acid, perchloric acid, perbenzoic acid, meta-chloroperoxybenzoic acid (mCPBA), trifluoroperacetic acid, periodic acid, magnesium monoperoxyphthalate, dimethyl dioxirane (DMDO), tert-butyl hydroperoxide, sodium hypochlorite, sodium tungstate, sodium periodate, potassium periodate, iodosyl benzene, pentafluoroiodosyl benzene, cumene hydroperoxide, potassium persulfate, potassium monoperoxysulfate, pyridine N-oxide, 2,6-dichloropyridine N-oxide, sodium chlorite, sodium hypochlorite, sodium chlorate, sodium perchlorate, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries);
2.97 Method 2.96, wherein the oxidizing agent is hydrogen peroxide, peracetic acid, trifluoroperacetic acid, meta-chloroperoxybenzoic acid, tert-butyl hydroperoxide, or potassium monoperoxysulfate;
2.98 Any of Methods 2.95-2.96, wherein the reaction is carried out using 1.0 to 5.0 equivalents of the oxidizing agent, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents; and optionally 0.01 to 1.0 equivalents of any one or more additional reagents (e.g., secondary oxidant, or catalyst, or ligand, or other agent), e.g., 0.01 to 0.5 equivalents, 0.01 to 0.2 equivalents, or 0.01 to 0.1, or 0.01 to 0.05 equivalents;
2.99 Any of Methods 2.95-2.98, wherein the acid is a Bronsted acid, e.g., selected from hydrochloric acid, perchloric acid, sulfuric acid, phosphoric acid, acetic acid, trifluoromethanesulfonic acid, methanesulfonic acid, trifluoroacetic acid, and nitric acid, or a heteropoly acid (e.g., phosphotungstic acid);
2.100 Method 2.99, wherein the acid is selected from sulfuric acid, phosphoric acid, and nitric acid;
2.101 Any of Methods 2.95-2.100, wherein the reaction is carried out using 1.0 to 5.0 equivalents of the acid, e.g., 1.0 to 4.0 equivalents, or 1.0 to 3.0 equivalents, or 1.0 to
2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents;
2.102 Any of Methods 2.95-2.101, wherein the reaction is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
2.103 Method 2.102, wherein the solvent is acetic acid and/or acetic anhydride, and optionally wherein the reaction further comprises an acetate salt (e.g., sodium or potassium acetate);
2.104 Any of Methods 2.94-2.103, wherein the reaction is carried out using a combination of hydrogen peroxide, sulfuric acid, and acetic acid, optionally 1.5-4 equivalents of each (e.g., 2-3 equivalents of each);
2.105 Any of Methods 2.94-2.103, wherein the reaction is carried out using a combination of acetic acid, peracetic acid, and sulfuric acid, optionally 1-3 equivalents of each (e.g., 1-1.5 equivalents of each);
2.106 Any of Methods 2.94-2.103, wherein the reaction is carried out using a combination of hydrogen peroxide, acetic acid, peracetic acid, and sulfuric acid, optionally 0.8-5 equivalents of each (e.g., 1-3 equivalents of each), optionally with sodium acetate (e.g., 0.05-0.24 equivalents);
2.107 Any of Methods 2.94-2.103, wherein the reaction is carried out using a combination of hydrogen peroxide, acetic anhydride, acetic acid, and sulfuric acid, optionally 0.8-5 equivalents of each (e.g., 1-3 equivalents of each), optionally with sodium acetate (e.g., 0.05-0.24 equivalents), further optionally with heptane co-solvent;
2.108 Any of Methods 2.94-2.103, wherein the reaction is carried out using a combination of potassium monoperoxysulfate, potassium hydrogen sulfate and potassium sulfate (e.g., Oxone®), in organic solvent (e.g., acetone/ethyl acetate), optionally 1-3 equivalents of potassium monoperoxysulfate (e.g., 1-2 equivalents);
2.109 Any of Methods 2.94-2.108, wherein the reaction is carried out at a temperature of 0° C. to 200° C., e.g., 0° C. to 150° C., or 0° C. to 100° C., or 25° C. to 200° C., or 25° C. to 150° C., or 25° C. to 100° C., or 50° C. to 200° C., or 50° C. to 150° C., or 50° C. to 100° C., or 75° C. to 200° C., or 75° C. to 150° C., or 75° C. to 100° C., or 80° C. to 150° C., or 80° C. to 100° C., or 90° C. to 150° C., or 90° C. to 125° C., or 90° C. to 110° C., or 90° C. to 100° C., or 90° C. to 95° C.;
2.110 Any of Methods 2.94-2.108, wherein the reaction is carried out at a temperature of 80° C. to 90° C., 20° C. to 70° C., 40° C. to 90° C., 20° C. to 50° C., or 20° C. to 30° C.;
2.111 Any of Methods 2.94-2.110, wherein the reactions steps (A), (B), (C), and/or (D) are carried out in a single vessel in a single reaction step, e.g., wherein step (B) spontaneously follows step (A), and step (C) spontaneously follow step (B), and step (D) spontaneously follows step (C);
2.112 Any of Methods 2.94-2.110, wherein the reactions steps (A), (B), (C), and/or (D) are carried out in a single vessel in a two reaction steps without isolation of any intermediates, e.g., wherein at least a second oxidizing agent is added in the second reaction step to initiate step (C), and wherein step (B) spontaneously follows step (A) and step (D) spontaneously follows step (C);
2.113 Any of Methods 2.94-2.112, wherein R is H, and wherein the reactant Compound (7) proceeds to the product Compound (6) in a single vessel, wherein the reaction is carried out by treating the Compound (7) with a combination of hydrogen peroxide, sulfuric acid, and acetic acid, optionally 1.5-4 equivalents of each (e.g., 2-3 equivalents of each, or 2 equivalents of each), optionally at a temperature of 75° C. to 150° C., or 80° C. to 100° C., or 90° C. to 95° C.;
2.114 Method 2, or any of 2.1-2.113, wherein the method further comprises a Step (E) of treating the Compound (6) with a base to cause enrichment of the Compound (6a) by isomerization of the Compound (6b) to the Compound (6a);
2.115 Method 2.114, wherein the base is selected from sodium hydride, potassium hydride, hydroxide bases (e.g., sodium hydroxide, potassium hydroxide), alkoxide bases (e.g., sodium tert-butoxide, potassium tert-butoxide, sodium methoxide, potassium methoxide, sodium ethoxide, potassium ethoxide), carbonate bases (e.g., sodium carbonate, potassium carbonate, cesium carbonate), bicarbonate bases (e.g., sodium bicarbonate), amide bases (e.g., lithium amide, lithium 2,2,6,6-tetramethylpiperide (LiTMP), lithium diisopropylamide (LDA), lithium bis(trimethylsilyl)amide) (LiHMDS)), alkyl lithium bases (e.g., sec-butyl lithium, tert-butyl lithium), and amine bases (e.g., triethylamine, diisopropylethylamine, N-methyl morpholine, DBU, DBN, pyridine, dimethylaminopyridine, imidazole);
2.116 Method 2.115, wherein the base is an alkoxide base (e.g., sodium tert-butoxide, potassium tert-butoxide, sodium methoxide, potassium methoxide, sodium ethoxide, potassium ethoxide) or an amide base (e.g., lithium amide, lithium 2,2,6,6-tetramethylpiperide (LiTMP), lithium diisopropylamide (LDA), lithium bis(trimethylsilyl)amide) (LiHMDS));
2.117 Method 2.115, wherein the base is sodium tert-butoxide or potassium tert-butoxide;
2.118 Any of Methods 2.114-2.117, wherein step (E) is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), and ethers (e.g., diethyl ether, diisopropyl ether, methyl tert-butyl ether, tetrahydrofuran, 1,4-dioxane, dimethoxyethane, diethylene glycol dimethyl ether);
2.119 Method 2.118, wherein step (E) is carried out in a hydrocarbon solvent, e.g., pentane, hexane, heptane, or cyclohexane.
2.120 Any of Methods 2.114-2.119, wherein step (E) is carried out at a temperature of −78° C. to 200° C., e.g., −50° C. to 150° C., or −25° C. to 100° C., or 0 to 75° C., or 15° C. to 50° C., or 20° C. to 30° C., −78° C. to 0° C., or −50° C. to 0° C., or −25° C. to 25° C., or 25° C. to 75° C., or 50° C. to 100° C., or 75° C. to 150° C., or 100° C. to 150° C., or 125° C. to 200° C., or 150° C. to 200° C.;
2.121 Any of Methods 2.114-2.120, wherein Step (E) provides an enrichment in the amount of the isomer (6a) of at least 10% by weight of the total weight of Compound (6), e.g., at least 15%, or at least 20%, or at least 25%;
2.122 Any of Methods 2.114-2.121, wherein Step (E) provides a product having a ratio of isomer (6a) to isomer (6b) of at least 90:10, e.g., at least 92:8, or at least 93:7, or at least 94:6, or at least 95:5;
2.123 Method 2, or any of 2.1-2.122, wherein the Method further comprises the crystallization of the product Compound (6) from a hydrocarbon solvent (e.g., pentane, hexane, heptane, cyclohexane, or a mixture thereof) at a temperature of less than 30° C., e.g., less than 25° C., or less than 15° C., or less than 10° C., or less than 5° C., or less than 0° C. to, or less than ˜5° C., or less than ˜10° C., or less than ˜20° C., or less than ˜30° C., or less than ˜40° C., or less than ˜50° C., e.g., as low as −78° C.;
2.124 Method 2.123, wherein the recrystallization step provides a product having a ratio of isomer (1a) to isomer (2a) of at least 95:5, e.g., at least 97:3, or at least 98:2, or at least 99:1, or at least 99.5:0.5;
2.125 Any of Methods 2.114-2.124, wherein the Method comprises isomerization step (E) followed by recrystallization of the product (6);
2.126 Method 2.125, wherein the isomerization step (E) and the recrystallization step are performed as a continuous process in which each recrystallization is followed by an isomerization followed by a recrystallization, etc., until a desired purity of the isomer (6a) is obtained;
2.127 Method 2.126, wherein the continuous isomerization and recrystallization steps provide a product having a ratio of isomer (6a) to isomer (6b) of at least 95:5, e.g., at least 97:3, or at least 98:2, or at least 99:1, or at least 99.5:0.5;
2.128 Method 2, or any of 2.1-2.127, wherein the method does not comprise the use of any reagents or reactants other than the Compound (7), and the reagents set forth herein (e.g., acids, bases, oxidizing agents, catalysts, protecting agents, deprotecting agents, solvents), for example, the method does not comprise the use of any carbon monoxide, carbonyl equivalents, or enzymes).

It is understood that the Method 2 is generic to the more specific Method 1, because the compounds (1) to (5) are species of the compounds (6) to (10), respectively.

In further embodiments of Method 2, the present disclosure provides Method 2, or any of 2.1-2.128, wherein step (B) provides as a by-product, one or more of the compounds of Formula XIII, XIV and XV:

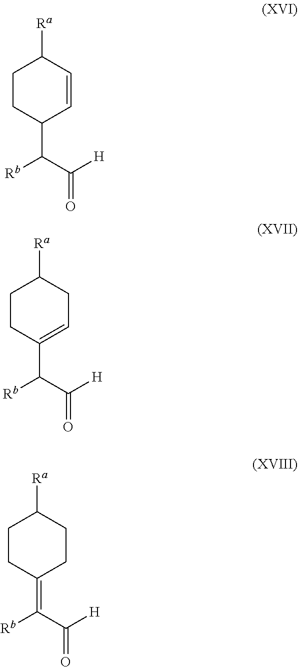

In particular embodiments, the present disclosure provides Method 2, or any of 2.1-2.128, wherein step (B) provides as a by-product, one or more of the compounds of Formula XVIa, XVIb, XIIa, XIIb, and XVIIIa:

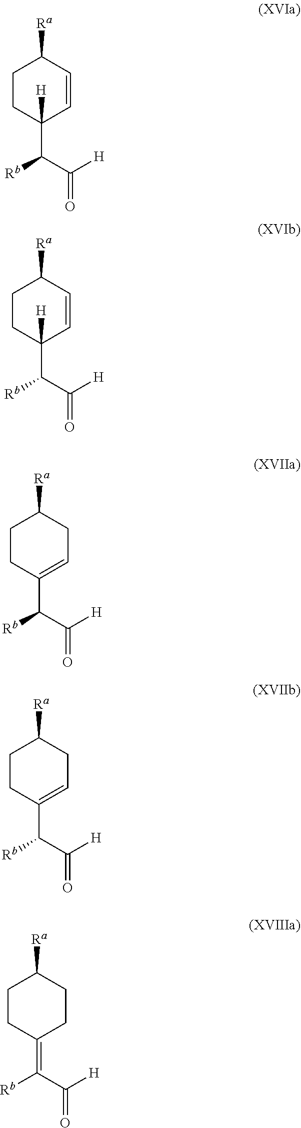

Without being bound by theory, it is believed that the double bond of the compounds according to Formulas XVI, XVII, XVIII, XVIa, XVIb, XVIIa, XVIIb, and/or XVIIIa, may form during step (A) and/or step (B) of Method 2 et seq., such as, by way of acid-catalyzed, base-catalyzed, or thermal elimination of the hydroxy group (—OH) or protected hydroxy group (—OR) of one or more of the compounds of Formula (7), (8), (8a), (8b), (9), (9a), or (9b). This may be particularly likely when the group R is —S(O)₂—R¹, for example, wherein R¹ is methyl, trifluoromethyl, phenyl, or tolyl. Thus, for example, the following reactions may occur during steps (A) and (B):

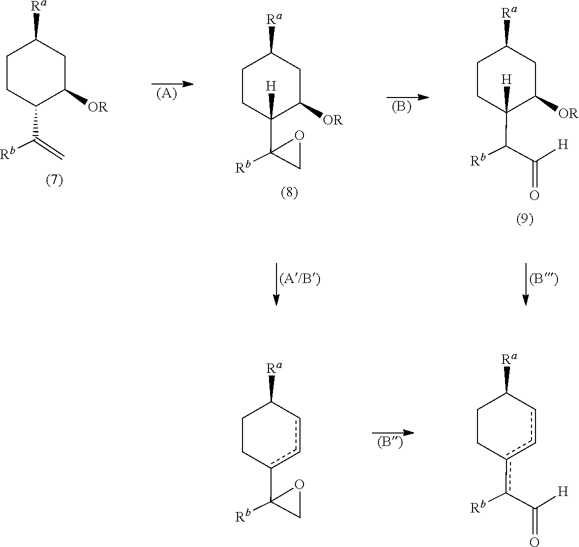

As shown above, under the conditions of step (A), the initially formed compound (8) may undergo elimination of OR to form the unsaturated epoxide compound (step A′ above), followed by, under the conditions of step (B), the conversion of the unsaturated epoxide compound to the unsaturated aldehyde (step B″ above). Alternatively, both of these conversions may occur under the conditions of step B (step B′ followed by step B″ above). Alternatively, under the conditions of step (B), the compound (9) may undergo elimination of OR to form the unsaturated epoxide compound (step B′″ above).

Optionally, the compound of Formulas XVI, XVII, XVIII, XVIa, XVIb, XVIIa, XVIIb, and/or XVIIIa, may be isolated (e.g., extracted, distilled, or separated via chromatography) from the product mixture of step (B).

It is understood that in reference to “steps” or “synthetic steps” the present disclosure recites specific chemical transformations which may optionally be accompanied by various procedural steps known to those skilled in the art, such as the stepwise addition of reagents, heating steps, cooling steps, quenching steps, precipitation steps, mixing steps, drying steps, evaporation steps, and other steps of purification (e.g., aqueous extraction, chromatography, distillation) and steps of analysis (e.g., thin-layer chromatography, MS, LCMS, NMR, elemental analysis, etc.). In embodiments wherein the invention of the present disclosure is limited to specified steps, it is understood that such limitation applies to the synthetic steps carried out and no limitation is made on the procedural steps involved in carrying out the method except where specifically indicated. The term “reaction steps” refers to the number of procedural steps carried out involving the addition of distinct selections of reagents.

In some embodiments, the mechanistic steps (A), (B), (C), and (D) may be combined into only two reaction steps. Without being bound by theory, it is believed that this may be accomplished because step (B) can occur spontaneously under thermal or acid-catalyzed conditions promoted by the reaction conditions of step (A), and likewise, step (D) can occur spontaneously under thermal or acid-catalyzed conditions promoted by the reaction conditions of step (C). Thus, a two-step reaction sequence may be employed in which oxidizing agent, optionally acid, and solvent are added in a first reaction step (e.g., hydrogen peroxide or peracetic acid, in sulfuric acid and/or acetic acid and/or acetic anhydride), followed after a period of time by a second oxidizing agent, an acid and solvent (e.g., hydrogen peroxide or peracetic acid, in sulfuric acid and/or acetic acid and/or acetic anhydride). This two-step sequence may be carried out in two reaction vessels or in a single reaction vessel (i.e., sequential addition of the two sets of reagents separated in time). When the two-step sequence is used, either the reaction mixture of the first step can be added to the reagents and solvents for the second step, or vice versa.

In a further embodiment of the first and second aspects, it has been unexpectedly found that a single vessel (“one-pot”) procedure can be used to convert the Compound (2) or (7) to the Compound (1) or (6), respectively, by treating the Compound (2) or (7) with an oxidizing agent, and an acid or a base, in a suitable solvent (e.g., an aqueous solvent). Thus, a single set of reagents are added and all mechanistic steps are carried out in one “reaction step.” In a preferred embodiment, the oxidizing agent is aqueous hydrogen peroxide, the acid is sulfuric acid (e.g., 60% aq.) and the solvent is acetic acid. Without being bound by theory, it is believed that the hydrogen peroxide and the acetic acid solvent may form peracetic acid in-situ. In another preferred embodiment, the reaction may be conducted using peracetic acid, trifluoromethanesulfonic acid, and acetic acid. In another preferred embodiment, the oxidizing agent is peracetic acid, the acid is sulfuric acid (e.g., 60% aq.) and the solvent is acetic acid and/or acetic anhydride. In some embodiments an acetate salt is added adjust the acidity of the reaction or to provide a buffering effect, for example, sodium acetate or potassium acetate.

In the one-pot procedure described herein, it is believed that the reactions proceed as described hereinabove. However, without being bound by theory, it is also believed that the reactions may also proceed via other intermediates. For example, for the case of making the compound (1):

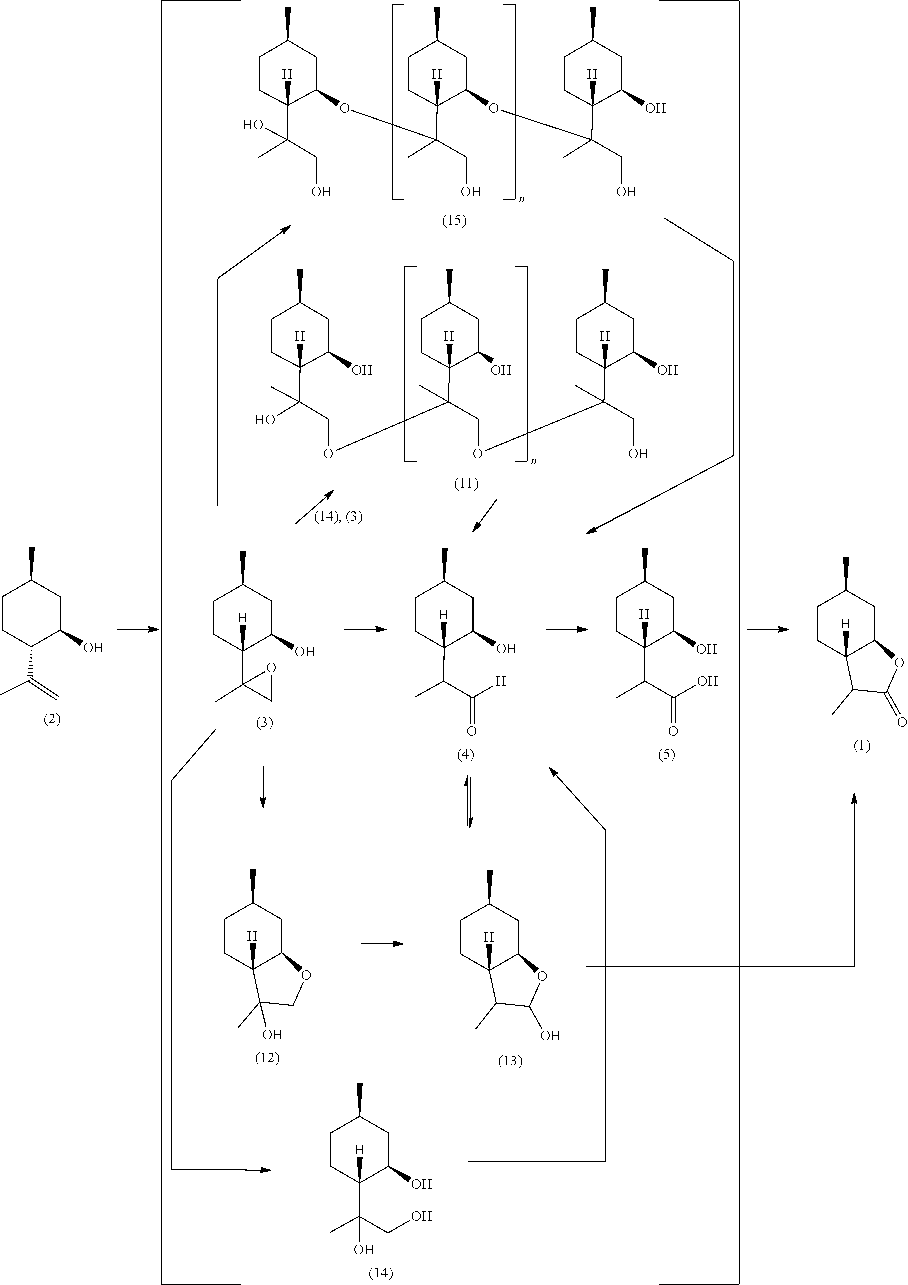

As shown above, under the one-pot reaction conditions described herein (e.g., hydrogen peroxide, sulfuric acid, acetic acid or peracetic acid, trifluoromethanesulfonic acid, acetic acid), the initially formed epoxide (3) may undergo an acid-catalyzed polymerization with intermediate (14) to form short oligomers (11), which may undergo oxidative depolymerization to form aldehydes (4). In addition, the initially formed epoxide (3) may undergo an acid-catalyzed self-polymerization to form short oligomers (15), which may also undergo oxidative depolymerization to form aldehydes (4) In addition, the epoxide (3) may undergo an acid-catalyzed intramolecular ring closure to form tertiary alcohol (12) which may rearrange to cyclic hemiacetal (13). This hemiacetal is reversibly formed by intramolecular condensation of aldehyde (4), or may be oxidized directly to the lactone product (1). In addition, epoxide (3) may undergo acid-catalyzed hydrolysis to form vicinal diol (14), which may undergo acid-catalyzed elimination to form aldehyde (4) (initially as its enol tautomer). While many of these individual steps are indicated as being acid-catalyzed, without being bound by theory, it is believed that many of them may also take place under base-catalyzed conditions. Thus, without being bound by theory, it is believed that mechanisms taking place in the one-pot procedure may be catalyzed by acid or base, or may be autocatalytic, in a polar medium (e.g., a medium comprising at least one polar solvent, e.g., water, an alcohol, an amine, a thiol, or other polar protic or polar aprotic solvents).

The corresponding reactions and intermediates as shown in the scheme above are likewise provided for the one-pot conversion of compound (7) to compound (6). Such a scheme would be substantially the same, except for having the compounds and intermediates (6), (7), (8), (9), and (10), as hereinbefore described, as well as the following additional intermediates (16), (17), (18), (19), and (20):

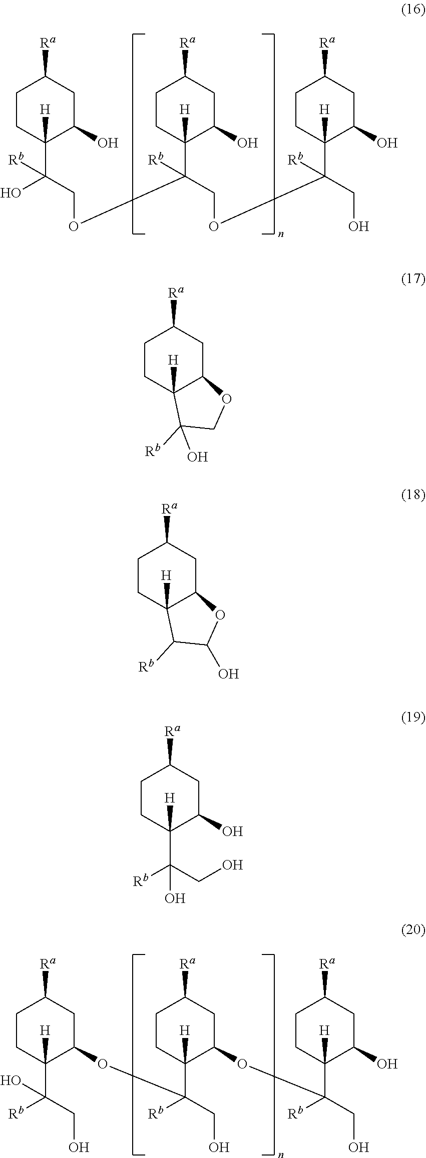

In another embodiment, using the conditions and procedures as described herein throughout, the present disclosure also provides methods for making compound (1) starting from any one of intermediates (3), (4), (5), (11), (12), (13), (14), or (15), by using appropriate reagents and conditions as would be clear from the present disclosure. Similarly, the present disclosure also provides for making compound (6) starting from any one of intermediates (8), (9), (10), (16), (17), (18), (19) or (20).

Therefore, in a third aspect, the present disclosure provides a method (Method 3) of making a compound (1) from compound (2), or of making compound (6) from compound (7):

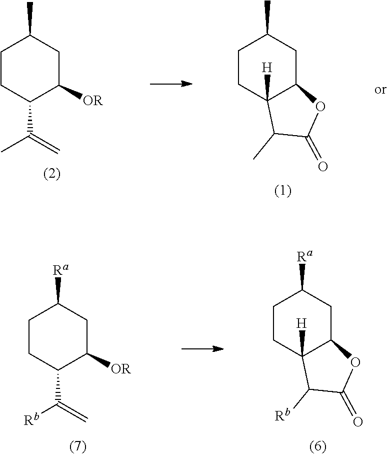

the method comprising the step of treating the compound (2) or (7) with an oxidizing agent, and optionally an acid or base, in a suitable solvent (e.g., an aqueous solvent), wherein the reaction proceeds in a single vessel without the isolation of any intermediates (e.g., a one-pot reaction);

wherein R is H or a protecting group (e.g., an ether protecting group, an ester protecting group, or a silyl ether protecting group), and wherein R^{a} is selected from H, optionally substituted C₁₋₆alkyl, optionally substituted C₂₋₆alkenyl, optionally substituted C₂₋₆alkynyl, optionally substituted aryl, O—R^{c}, and —C(O)—R^{c}, and R^{b} is selected from H, optionally substituted C₁₋₆alkyl, optionally substituted aryl, O—R^{c}, and —C(O)—R^{c}, and R^{c} is selected from H, optionally substituted C₁₋₆alkyl, optionally substituted C₂₋₆alkenyl, optionally substituted C₂₋₆alkynyl, and optionally substituted aryl.

In further embodiments of the third aspect, the present disclosure provides:
3.1. Method 3, wherein R is H;
3.2. Method 3, wherein R is an ester protecting group, e.g., —C(O)—R¹ or —S(O)₂—R¹, and wherein R¹ is H, C₁₋₆alkyl (e.g., methyl or ethyl), haloC₁₋₆alkyl (e.g., chloromethyl or trifluoromethyl), C₁₋₆alkoxy (e.g., methoxy or ethoxy), C₁₋₆alkoxymethyl (e.g., methoxyethyl or ethoxymethyl), aryl (e.g., phenyl or tolyl), arylmethyl (e.g., benzyl), aryloxy (e.g., phenoxy), or aryloxymethyl (e.g., phenoxymethyl);
3.3. Method 3.2, wherein R is —C(O)—R¹, and wherein R¹ is methyl, ethyl, propyl, isopropyl, or tert-butyl, or wherein R is —S(O)₂—R¹, and wherein R¹ is methyl;
3.4. Method 3.2, wherein R is —C(O)—R¹, and wherein R¹ is methyl;
3.5. Method 3.2, wherein R is —C(O)—R¹, and wherein R¹ is chloromethyl, dichloromethyl, trichloromethyl, 2,2,2-trichloroethyl, trifluoromethyl, methoxymethyl, phenoxymethyl, or benzyl, or wherein R is —S(O)₂—R¹, and wherein R¹ is trifluoromethyl, phenyl or tolyl;
3.6. Method 3, wherein R is an ether protecting group, e.g., R is unsubstituted C₁₋₆alkyl (e.g., methyl, ethyl, isopropyl, tert-butyl), or substituted C₁₋₆alkyl, such as C₁₋₆alkoxy-C₁₋₆alkyl, aryloxy-C₁₋₆alkyl, or aryl-C₁₋₆alkyl (e.g., —CH₂—O-Me, —CH₂—O-Et, —CH₂—S-Me, —CH₂—O—CH₂CH₂—OMe, CH₂—O—CH₂CCl₃, CH₂—O—CH₂CH₂—SiMe₃, —CH₂—O-Ph, —CH₂—O—CH₂-Ph, —CH₂—O—CH₂-(4-methoxyphenyl), —CH₂—O—CH₂-(3,4-dimethoxyphenyl), —CH₂CH₂-OEt, —CH₂CH₂Si(Me)₃, —CH₂CCl₃, —CH₂-Ph, —CH₂-(4-methoxyphenyl), —CH₂-(3,4-dimethoxyphenyl), —CH₂-(2,6-dimethoxyphenyl), or tetrahydropyranyl;
3.7. Method 3, wherein R is a silyl ether protecting group (e.g., R is —Si(R²)(R³)(R⁴), wherein R², R³, and R⁴ are each independently selected from C₁₋₆alkyl (e.g., methyl, ethyl, isopropyl, tert-butyl, thexyl, benzyl), C₁₋₆alkoxy (e.g., methoxy, ethoxy, tert-butoxy), and aryl (e.g., phenyl);
3.8. Method 3.5, wherein R is selected from trimethylsilyl, triethylsilyl, triisopropylsilyl, dimethylisopropylsilyl, diethylisopropylsilyl, dimethylthexylsilyl, tert-butyldimethylsilyl, tert-butyldiphenylsilyl, tribenzylsilyl, triphenylsilyl, diphenylmethylsilyl, di-tert-butylmethylsilyl, tris(trimethylsilyl)silyl, tert-butylmethoxyphenylsilyl, and tert-butoxydiphenylsilyl;
3.9. Method 3 or any of 3.1 to 3.8, wherein the method comprises an Intermediate Compound (3), e.g., Compound (3a) or Compound (3b), or a mixture thereof, or wherein the method comprises an Intermediate Compound (8), e.g., Compound (8a) or Compound (8b), or a mixture thereof;
3.10. Method 3 or any of 3.1 to 3.9, wherein the method comprises an Intermediate Compound (4), e.g., Compound (4a) or Compound (4b), or a mixture thereof, or wherein the method comprises an Intermediate Compound (9), e.g., Compound (9a), or Compound (9b), or a mixture thereof;
3.11. Method 3 or any of 3.1 to 3.10, wherein the method comprises an Intermediate Compound (5), e.g., Compound (5a) or Compound (5b), or a mixture thereof, or wherein the method comprises an Intermediate Compound (10), e.g., Compound (10a) or Compound (10b), or a mixture thereof;
3.12. Method 3 or any of 3.1 to 3.11, wherein the Compound (1) is Compound (1a) or Compound (1b), or a mixture thereof, or wherein the Compound (6) is Compound (6a) or Compound (6b) or a mixture thereof;
   optionally, wherein the Compound (1) or the Compound (6) is enriched in one isomer or the other isomer, and optionally wherein the method further comprises the step of purifying or separating the isomers;
3.13. Method 3, or any of 3.1-3.12, wherein R^{a} is H;
3.14. Method 3, or any of 3.1-3.12, wherein R^{a} is optionally substituted C₁₋₆alkyl;
3.15. Method 3, or any of 3.1-3.14, wherein R^{b} is H;
3.16. Method 3, or any of 3.1-3.14, wherein R^{b} is optionally substituted C₁₋₆alkyl;
3.17. Method 3, or any of 3.1-3.16, wherein R^{a} and/or R^{b} is optionally substituted aryl;
3.18. Method 3, or any of 3.1-3.17, wherein R^{a} and/or R^{b} is O—R^{c};
3.19. Method 3, or any of 3.1-3.18, wherein R^{a} and/or R^{b} is —C(O)—R^{c};
3.20. Method 3.18 or 3.19, wherein R^{c} is selected from H, optionally substituted C₁₋₆alkyl, and optionally substituted aryl;
3.21. Method 3.18 or 3.19, wherein R^{c} is selected from H and optionally substituted C₁₋₆ alkyl;
3.22. Method 3, or any of 3.1-3.21, wherein the oxidizing agent is selected from one or more of hydrogen peroxide, a chromium oxidant (e.g., chromium trioxide, chromic acid, pyridinium chlorochromate, potassium dichromate, chromium trioxide-pyridine complex, pyridinium dichromate), osmium tetroxide, potassium permanganate, peracetic acid, perchloric acid, perbenzoic acid, meta-chloroperoxybenzoic acid (mCPBA), trifluoroperacetic acid, periodic acid, magnesium monoperoxyphthalate, dimethyl dioxirane (DMDO), tert-butyl hydroperoxide, sodium hypochlorite, sodium tungstate, sodium periodate, potassium periodate, iodosyl benzene, pentafluoroiodosyl benzene, cumene hydroperoxide, potassium persulfate, potassium monoperoxysulfate, pyridine N-oxide, 2,6-dichloropyridine N-oxide, sodium chlorite, sodium hypochlorite, sodium chlorate, sodium perchlorate, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries);
3.23. Method 3.22, wherein the oxidizing agent is hydrogen peroxide, peracetic acid, trifluoroperacetic acid, meta-chloroperoxybenzoic acid, tert-butyl hydroperoxide, or potassium monoperoxysulfate;
3.24. Method 3, or any of 3.1-3.23, wherein the reaction is carried out using 1.0 to 5.0 equivalents of the oxidizing agent, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents; and optionally 0.01 to 1.0 equivalents of any one or more additional reagents (e.g., secondary oxidant, or catalyst, or ligand, or other agent), e.g., 0.01 to 0.5 equivalents, 0.01 to 0.2 equivalents, or 0.01 to 0.1, or 0.01 to 0.05 equivalents;
3.25. Method 3, or any of 3.1-3.24, wherein the reaction comprises an acid catalyst;
3.26. Method 3.25, wherein the acid is a Bronsted acid, e.g., selected from hydrochloric acid, perchloric acid, sulfuric acid, phosphoric acid, acetic acid, peracetic acid, trifluoromethanesulfonic acid, methanesulfonic acid, trifluoroacetic acid, and nitric acid, or a heteropoly acid (e.g., phosphotungstic acid);
3.27. Method 3.26, wherein the acid is selected from sulfuric acid, phosphoric acid, trifluoromethanesulfonic acid, peracetic acid, and nitric acid;
3.28. Any of Methods 3.25-3.27, wherein the reaction is carried out using 1.0 to 5.0 equivalents of the acid, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents;
3.29. Method 3, or any of 3.1-3.24, wherein the reaction comprises a base catalyst;
3.30. Method 3.29, wherein the base is a Bronsted base, e.g., selected from an inorganic base (e.g., hydroxide, carbonate, or bicarbonate) or an organic base (e.g., an amine base);
3.31. Method 3.30, wherein the base is an inorganic base selected from lithium hydroxide, sodium hydroxide, potassium hydroxide, magnesium hydroxide, calcium hydroxide, lithium carbonate, sodium carbonate, potassium carbonate, lithium bicarbonate, sodium bicarbonate, and potassium bicarbonate;
3.32. Method 3.30, wherein the base is an amine base selected from triethylamine, diisopropylethylamine, N-methylpiperidine, N-methylmorpholine, pyridine, 4-dimethylaminopyridine, imidazole, N-methylimidazole, 1,5-diazabicyclo[4.3.0]non-5-ene (DBN), and 1,8-diazabicyclo[5.4.0]undec-7-ene (DBU);
3.33. Any of Methods 3.29-3.32, wherein the reaction is carried out using 1.0 to 5.0 equivalents of the base, e.g., 1.0 to 4.0 equivalents, or 1.0 to 3.0 equivalents, or 1.0 to
2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents;
3.34. Method 3, or any of 3.1-3.33, wherein the reaction is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof;
3.35. Method 3.34, wherein the solvent is acetic acid and/or acetic anhydride, optionally wherein the reaction further comprises an acetate salt (e.g., sodium or potassium acetate);
3.36. Method 3, or any of 3.1-3.35, wherein the reaction comprises a polar protic solvent (e.g., water, an alcohol, an amine, or a thiol), in any amount (e.g., as solvent or in a catalytic amount, e.g., less than 0.5 molar equivalents);
3.37. Method 3.36, wherein the reaction comprises water (e.g., an aqueous solvent mixture);
3.38. Method 3.37, wherein any water present in the reaction is provided by the reagents (e.g., aqueous hydrogen peroxide, aqueous sulfuric acid, etc.);
3.39. Method 3, or any of 3.1-3.35, wherein the reaction is non-aqueous (e.g., no water is present), optionally wherein at least one non-aqueous polar protic solvent is present (e.g., an alcohol, amine or thiol);
3.40. Method 3, or any of 3.1-3.35, wherein the reaction comprises no polar protic solvents and/or wherein the reaction is non-aqueous (e.g., no water is present);
3.41. Method 3, or any of 3.1-3.40, wherein the reaction is carried out using a combination of hydrogen peroxide, sulfuric acid, and acetic acid, optionally 1.5-4 equivalents of each (e.g., 2-3 equivalents of each);
3.42. Method 3, or any of 3.1-3.40, wherein the reaction is carried out using a combination of peracetic acid (e.g., 2-4 equiv. or about 3 equiv.), trifluoromethanesulfonic acid (e.g., 0.1-2 equiv., or about 0.5 equiv.), and acetic acid (e.g., 2-3 equiv., or about 2.5 equiv.);
3.43. Method 3, or any of 3.1-3.42, wherein the reaction is carried out at a temperature of 0° C. to 200° C., e.g., 0° C. to 150° C., or 0° C. to 100° C., or 0° C. to 75° C., or 0° C. to 50° C., or 0° C. to 25° C., or 25° C. to 200° C., or 25° C. to 150° C., or 25° C. to 100° C., or 25° C. to 75° C., or 25° C. to 50° C., or 25° C. to 30° C., or 50° C. to 200° C., or 50° C. to 150° C., or 50° C. to 100° C., or 75° C. to 200° C., or 75° C. to 150° C., or 75° C. to 100° C., or 80° C. to 150° C., or 80° C. to 100° C., or 90° C. to 150° C., or 90° C. to 125° C., or 90° C. to 110° C., or 90° C. to 100° C., or 90° C. to 95° C.;
3.44. Method 3, or any of 3.1-3.43, wherein R is H, and wherein the reactant Compound (2) or (7) proceeds to the product Compound (1) or (6) in a single vessel, wherein the reaction is carried out by treating the Compound (2) or (7) with a combination of hydrogen peroxide, sulfuric acid, and acetic acid, optionally 1.5-4 equivalents of each (e.g., 2-3 equivalents of each, or 2 equivalents of each), optionally at a temperature of 75° C. to 150° C., or 80° C. to 100° C., or 90° C. to 95° C., or with a combination of peracetic acid (e.g., 2-4 equiv. or about 3 equiv.), trifluoromethanesulfonic acid (e.g., 0.1-2 equiv., or about 0.5 equiv.), and acetic acid (e.g., 2-3 equiv., or about 2.5 equiv.), optionally at a temperature of 25° C. to 50° C., or 25° C. to 30° C.;
3.45. Method 3, or any of 3.1-3.44, wherein the method further comprises a Step (2) of treating the Compound (1) or (6) with a base to cause enrichment of one or more isomers of Compound (1) or (6) by isomerization;
3.46. Method 3.45, wherein the base is selected from sodium hydride, potassium hydride, hydroxide bases (e.g., sodium hydroxide, potassium hydroxide), alkoxide bases (e.g., sodium tert-butoxide, potassium tert-butoxide, sodium methoxide, potassium methoxide, sodium ethoxide, potassium ethoxide), carbonate bases (e.g., sodium carbonate, potassium carbonate, cesium carbonate), bicarbonate bases (e.g., sodium bicarbonate), amide bases (e.g., lithium amide, lithium 2,2,6,6-tetramethylpiperide (LiTMP), lithium diisopropylamide (LDA), lithium bis(trimethylsilyl)amide) (LiHMDS)), alkyl lithium bases (e.g., sec-butyl lithium, tert-butyl lithium), and amine bases (e.g., triethylamine, diisopropylethylamine, N-methyl morpholine, DBU, DBN, pyridine, dimethylaminopyridine, imidazole);
3.47. Method 3.46, wherein the base is an alkoxide base (e.g., sodium tert-butoxide, potassium tert-butoxide, sodium methoxide, potassium methoxide, sodium ethoxide, potassium ethoxide) or an amide base (e.g., lithium amide, lithium 2,2,6,6-tetramethylpiperide (LiTMP), lithium diisopropylamide (LDA), lithium bis(trimethylsilyl)amide) (LiHMDS));
3.48. Method 3.47, wherein the base is sodium tert-butoxide or potassium tert-butoxide;
3.49. Any of Methods 3.45-3.48, wherein step (2) is carried out in a solvent selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), and ethers (e.g., diethyl ether, diisopropyl ether, methyl tert-butyl ether, tetrahydrofuran, 1,4-dioxane, dimethoxyethane, diethylene glycol dimethyl ether);
3.50. Method 3.49, wherein step (2) is carried out in a hydrocarbon solvent, e.g., pentane, hexane, heptane, or cyclohexane.
3.51. Any of Methods 3.45-3.50, wherein step (2) is carried out at a temperature of −78° C. to 200° C., e.g., −50° C. to 150° C., or −25° C. to 100° C., or 0 to 75° C., or 15° C. to 50° C., or 20° C. to 30° C., −78° C. to 0° C., or −50° C. to 0° C., or −25° C. to 25° C., or 25° C. to 75° C., or 50° C. to 100° C., or 75° C. to 150° C., or 100° C. to 150° C., or 125° C. to 200° C., or 150° C. to 200° C.;
3.52. Any of Methods 3.41-3.51, wherein Step (2) provides an enrichment in the amount of isomer (1a), or isomer (6a), of at least 10% by weight of the total weight of Compound (1) or (6), e.g., at least 15%, or at least 20%, or at least 25%;
3.53. Any of Methods 3.45-3.52, wherein Step (2) provides a product having a ratio of isomer (1a) to isomer (1b), or of isomer (6a) to isomer (6b), of at least 90:10, e.g., at least 92:8, or at least 93:7, or at least 94:6, or at least 95:5;
3.54. Method 3, or any of 3.1-3.53, wherein the Method further comprises the crystallization of the product Compound (1) or (6), from a hydrocarbon solvent (e.g., pentane, hexane, heptane, cyclohexane, or a mixture thereof) at a temperature of less than 30° C., e.g., less than 25° C., or less than 15° C., or less than 10° C., or less than 5° C., or less than 0° C. to, or less than ˜5° C., or less than ˜10° C., or less than ˜20° C., or less than ˜30° C., or less than ˜40° C., or less than ˜50° C., e.g., as low as −78° C.;
3.55. Method 3.54, wherein the recrystallization step provides a product having a ratio of isomer (1a) to isomer (1b), or of isomer (6a) to isomer (6b), of at least 95:5, e.g., at least 97:3, or at least 98:2, or at least 99:1, or at least 99.5:0.5;
3.56. Any of Methods 3.45-3.55, wherein the Method comprises isomerization step (2) followed by recrystallization of the product (1) or (6);
3.57. Method 3.56, wherein the isomerization step (2) and the recrystallization step are performed as a continuous process in which each recrystallization is followed by an isomerization followed by a recrystallization, etc., until a desired purity of the isomer (1a) or of the isomer (6a) is obtained;
3.58. Method 3, or any of 3.1-3.57, wherein the method further comprises a preliminary synthetic step (a protection step) of converting Compound (2′), wherein R is H, to the Compound (2), wherein R is not H (e.g., wherein R is a protecting group):

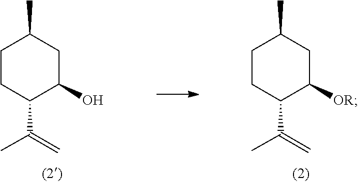

or of converting Compound (7′), wherein R is H, to the Compound (7), wherein R is not H (e.g., wherein R is a protecting group):

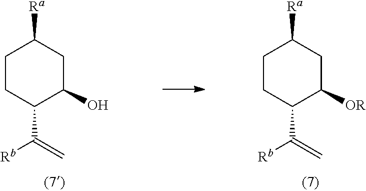

3.59. Method 3.58, wherein the protection step immediately precedes the step of treating the Compound (2) or (7) with an oxidizing agent, an acid or base, and a suitable aqueous solvent;
3.60. Method 3.58 or 3.59, wherein the protection step is carried out by treating the compound (2′) or (7′) with a suitable protecting agent in a suitable solvent, optionally with a suitable base;
3.61. Method 3.60, wherein the protecting agent is selected from an acyl halide (e.g., acetyl chloride, benzoyl chloride, chloroacetyl chloride, dichloroacetyl chloride, trichloroacetyl chloride, methoxyacetyl chloride, phenoxyacetyl chloride, pivaloyl chloride, benzoyl chloride), an acyl anhydride (e.g., acetic anhydride, chloroacetic anhydride, dichloroacetic anhydride, trichloroacetic anhydride, trifluoroacetic anhydride, methoxyacetyl anhydride, phenoxyacetyl anhydride, pivaloyl anhydride, benzoyl anhydride), an alkyl halide (e.g., methoxymethyl chloride, methoxymethyl bromide, methoxyethyl chloride, methylthiomethyl iodide, benzyloxymethyl chloride, 4-methoxybenzyloxymethyl chloride, 2-methoxyethoxymethyl chloride, 2,2,2-trichloroethoxymethyl chloride, 2-trimethylsilylethoxymethyl chloride, 4-methoxybenzyl chloride, 4-methoxybenzyl bromide, 3,4-dimethoxybenzyl bromide,), a silyl reagent (e.g., the chloride, silane or triflate of the group trimethylsilyl, triethylsilyl, triisopropylsilyl, dimethylisopropylsilyl, diethylisopropylsilyl, dimethylthexylsilyl, tert-butyldimethylsilyl, tert-butyldiphenylsilyl, tribenzylsilyl, triphenylsilyl, diphenylmethylsilyl, di-tert-butylmethylsilyl, tris(trimethylsilyl)silyl, tert-butylmethoxyphenylsilyl, and tert-butoxydiphenylsilyl), or other protecting agents (e.g., formic acid, acetic acid, ethyl formate, methyl formate, chloroacetic acid, dihydropyran, 2-hydroxytetrahydropyran, ethyl vinyl ether, trimethylsilylethoxyethene, isobutylene, methanesulfonyl chloride, trifluoromethanesulfonyl chloride, trifluoromethanesulfonic anhydride, N,N-bis(trifluoromethanesulfonyl)aniline, benzenesulfonyl chloride, toluenesulfonyl chloride);
3.62. Method 3.60 or 3.61, wherein the suitable base is selected from sodium hydride, potassium hydride, hydroxide bases (e.g., sodium hydroxide, potassium hydroxide), alkoxide bases (e.g., sodium tert-butoxide, potassium tert-butoxide), carbonate bases (e.g., sodium carbonate, potassium carbonate, cesium carbonate), bicarbonate bases (e.g., sodium bicarbonate), and amine bases (e.g., triethylamine, diisopropylethylamine, N-methyl morpholine, DBU, DBN, pyridine, dimethylaminopyridine, imidazole);
3.63. Any of methods 3.60-3.62, wherein the suitable solvent is selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid), carbon dioxide (e.g., supercritical carbon dioxide), and carbon disulfide, or combinations thereof, or wherein the solvent is a neat reagent (e.g., pyridine);
3.64. Any of Methods 3.60-3.63, wherein the protection step is carried out using 1.0 to 5.0 equivalents of the protecting agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents;
3.65. Any of Methods 3.60-3.64, wherein the protection step is carried out at a temperature of −100° C. to 200° C., e.g., −50° C. to 150° C., or −100° C. to 0° C., or −100° C. to −50° C., or −50° C. to 0° C., or −25° C. to 0° C., or −25° C. to 25° C., or 0° C. to 200° C., or 0° C. to 150° C., or 0° C. to 100° C., or 0° C. to 50° C., or 0° C. to 25° C., or 25° C. to 150° C., or 25° C. to 100° C., or 50° C. to 200° C., or 50° C. to 100° C., or 75° C. to 200° C., or 100° C. to 200° C., or 150° C. to 200° C.;
3.66. Method 3, or any of 3.1-3.65, wherein the method proceeds through a further intermediate step (D′) of converting Intermediate Compound (5′), wherein R is not H (e.g., wherein R is a protecting group), to Intermediate Compound (5″), wherein R is

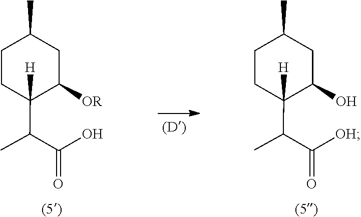

or of converting Intermediate Compound (10′), wherein R is not H (e.g., wherein R is a protecting group), to Intermediate Compound (10″), wherein R is H:

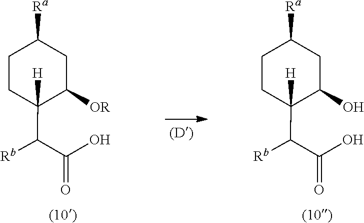

wherein the protecting group R is spontaneously eliminated during method (i.e., no synthetic deprotection step D′ is needed);
3.67. Method 3, or any of 3.1-3.66, wherein the method does not comprise any synthetic steps and/or mechanistic steps other than the step of treating the Compound (2) or the Compound (7) with an oxidizing agent, an acid or a base, and a suitable solvent or solvents, and optionally the protection step, one or more isomerization steps (2), and/or one or more crystallization steps;
3.68. Method 3, or any of 3.1-3.67, wherein the method does not comprise the use of any reagents or reactants other than the Compound (2) or the Compound (7), and the reagents set forth herein (e.g., acids, bases, oxidizing agents, catalysts, protecting agents, deprotecting agents, solvents), for example, the method does not comprise the use of any carbon monoxide, carbonyl equivalents, or enzymes).

It is understood that the Method 3 is a more specific variation of Methods 1 and 2, because all three methods proceed from the same ultimate starting materials, to the same final products, and may proceed through the same mechanistic steps, but in Method 3 the process is simplified by performing all of the steps in a one-pot process.

As used herein, the term “one-pot” refers to a process which can be involve one reaction step, two reaction steps, or more reaction steps, as long as all reaction components are present together (aside from when reagents are being added) and no purification step is performed between the reaction steps (e.g., no aqueous work-up, extraction, filtration, precipitation, chromatography, or distillation, or any other procedures which would remove any components from the reaction mixture). A one-pot procedure can involve adding all reagents at the outset of the process and allowing all necessary mechanistic steps to take place through completion of the process (i.e., a one-reaction step process), or the one-pot procedure can involve adding one set of reagents at the outset and a different set of reagents later, whereby it is expected that only some of the mechanistic steps take place after the first reagent addition(s) and the remaining reaction steps take place after the final reagent addition(s).

Therefore, as used herein, the term “one-pot” refers to the fact that multiple mechanistic or reaction steps occur without purification, not necessarily that only a single reaction vessel is involved, although this may be the case. For example, where the “one-pot” process involves two reaction steps, it may be necessary to involve a second reaction vessel. Thus, in the two-reaction step process, the reagents for the second reaction step may be added directly to the reaction mixture from the first reaction step. In such case, only a single reaction vessel is necessary. However, alternatively, the reaction mixture from the first reaction step may be added directly to the reagents for the second reaction step, which are then necessarily in a second vessel initially. But upon completion of this addition, the reaction again involves a single vessel holding all of the reagents, intermediates, reactants, and products from all steps, thus making this still a “one-pot” procedure.

The Compounds (1a) and (6a) of the products described in the present disclosure may be preferred commercially due to their more favorable olfactory effects compared to the isomeric Compounds (1b) and (6b). Advantageously, Method 1 et seq., Method 2 et seq., and Method 3 et seq., provide an improvement over the prior art in that the methods tend to provide an excess of the isomers (1a) and (6a) (i.e., a racemic product is not usually obtained). In some embodiments of the present disclosure, the preference for the isomers (1a) and (6a) may be further enhanced by subjecting the initial products to a base-catalyzed isomerization reaction, such as, by treating the initial product (1) or (6) with sodium tert-butoxide in a hydrocarbon solvent, or similar methods. This can substantially increase the amount of the isomers (1a) and (6a) produced by the method.

In some embodiments of the present disclosure, the preference for the isomers (1a) and (6a) may be further enhanced by subjecting the initial products to a crystallization procedure which selectively crystalizes the isomers (1a) and (6a). For example, the initial product (1) or (6) may be crystallized from a hydrocarbon solvent at a temperature under 0° C. Optionally, by performing the Method 1 or the Method 2 or the Method 3 with a base-catalyzed isomerization step and a crystallization step, product (1) or product (6) can be obtained having 99% or more of the (1a) or (6a) isomer, respectively.

In a fourth aspect, the present disclosure provides Compound (1) made according to Method 1 or any of 1.1 et seq. or Method 3 or any of 3.1 et seq. In some embodiments, the Compound (1) is the isomer (1a), or the isomer (1b) or a mixture thereof:

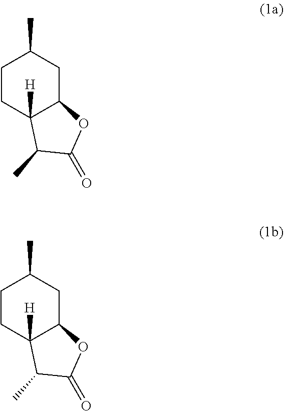

In some embodiments, the Compound (1) is enriched in one isomer or the other isomer. In some embodiments, the isomers are separated and purified.

In another embodiment of the fourth aspect, the present disclosure provides Compound (6) made according to Method 2 or any of 2.1 et seq. or Method 3 or any of 3.1 et seq. In some embodiments, the Compound (6) is the isomer (6a), or the isomer (6b) or a mixture thereof:

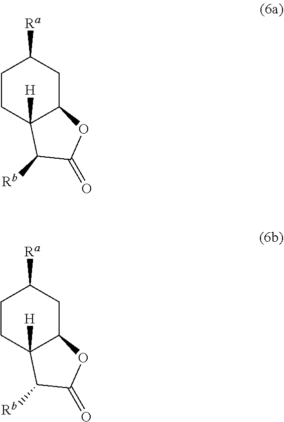

In some embodiments, the Compound (6) is enriched in one isomer or the other isomer. In some embodiments, the isomers are separated and purified.

In another embodiment of the fourth aspect, the present disclosure provides any one or more of the compounds according to Formulas XIII, XIV, XV, XIIIa, XIIIb, XIVa, XIVb, and/or XVa, made according to Method 1, or any of 1.1 et seq., for example, wherein said compounds is/are isolated (e.g., extracted, distilled, or separated via chromatography) from the product mixture of step (B).

In another embodiment of the fourth aspect, the present disclosure provides any one or more of the compounds according to Formulas XVI, XVII, XVIII, XVIa, XVIb, XVIIa, XVIIb, and/or XVIIIa, made according to Method 2, or any of 2.1 et seq., for example, wherein said compounds is/are isolated (e.g., extracted, distilled, or separated via chromatography) from the product mixture of step (B).

In a fifth aspect, the present disclosure provides a method (Method 4) of making a compound according to Formula XIII, XIV or XV, comprising the steps (A) and (B) as recited in Method 1, or any of 1.1-1.115, as applicable. In some embodiments, the method is a method of making a compound according to Formula XIIIa, XIIIb, XIVa, XIVb, and/or XVa.

In another embodiment of the fifth aspect, the present disclosure provides a method (Method 5) of making a compound according to Formula XVI, XVII, XVIII, comprising the steps (A) and (B) as recited in Method 2, or any of 2.1-2.128, as applicable. In some embodiments, the method is a method of making a compound according to Formula XVIa, XVIb, XVIIa, XVIIb, and/or XVIIIa.

In a sixth aspect, the present disclosure provides a composition comprising a compound of Formula XIII, XIV or XV, XIIIa, XIIIb, XIVa, XIVb, and/or XVa, or a composition comprising Compound (1), made according to Method 1 or any of 1.1 et seq. or Method 3 or any of 3.1 et seq., or Compound (6), made according to Method 2 or any of 2.1 et seq. or Method 3 or any of 3.1 et seq., wherein said compound imparts a flavor or fragrance to the composition. In some embodiments, the composition is a flavor or fragrance composition, for example, further comprising one or more additional flavor or fragrance agents or additives, and at least one solvent, or carrier. The composition may be a liquid or solid composition, such as a soft or waxy solid. The composition may further comprise one or more excipients, such as polymers, gelling agents, powdery substrates, surfactants, emollients, plasticizers, wetting agents, swelling agents, or active agents (e.g., an oral care active or a medicinal active agent), or any other cosmetically acceptable or orally acceptable additives. In some embodiments said compound, or said composition, is used to impart a flavor or fragrance to a product, such as a consumer product.

Suitable solvents for said compositions include: water, methanol, ethanol, propanol, isopropanol, butanol, 3-methoxy-3-methyl-1-butanol, benzyl alcohol, ethyl carbitol (diethylene glycol monoethyl ether), dimethyl ether, diethyl ether, diisopropyl ether, methyl tert-butyl ether, ethylene glycol, propylene glycol, dipropylene glycol, butylene glycol, hexylene glycol, glycerin, ethylene glycol monomethyl ether, propylene glycol monomethyl ether, propylene glycol monoethyl ether, dipropylene glycol monomethyl ether, isoparaffin, paraffin, limonene, pinene, triethyl citrate, triacetin, benzyl benzoate, isopropyl myristate, triglycerides, liquid waxes, propylene glycol derivatives, ethylene glycol derivatives, other alcohols or ethers, or any combination thereof.

The inventors have also surprisingly found that the highly convenient one-pot one- or two-reaction step procedure, described hereinabove, which provides for double bond epoxidation, epoxide rearrangement, and aldehyde oxidation, is a versatile method of converting a double bond to a carboxylic acid without loss of a carbon atom (which differentiates it from direct double ozonolysis or oxidative cleavage). The inventors have therefore discovered that this process can be used to form a variety of carboxylic acids directly from alkenes without proceeding through an oxidative cleavage reaction or hydrolysis reaction.

Therefore, in a seventh aspect, the present disclosure therefore provides, a method (Method 7) of making a carboxylic acid compound (14) from an alkene compound (11), wherein the method proceeds through intermediate compounds (12) and (13), which optionally are generated in-situ and not isolated or purified:

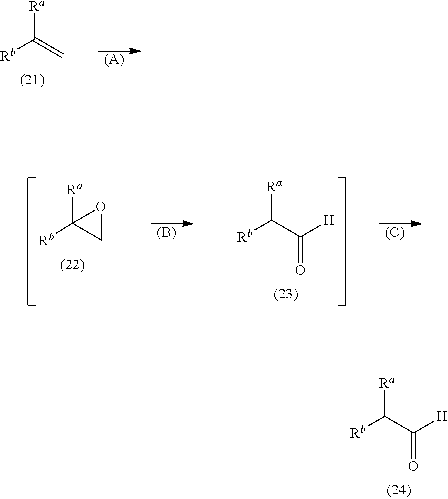

wherein the method comprises the following mechanistic steps;

(A) epoxidizing alkene Compound (21), to form epoxide Compound (22);

(B) rearranging epoxide Compound (22) to form aldehyde Compound (23); and

(C) oxidizing aldehyde Compound (23) to form carboxylic acid Compound (24);

wherein R^{a} and R^{b} are each independently selected from H, optionally substituted C₁₋₃₀ alkyl, optionally substituted C₃₋₁₀cycloalkyl, optionally substituted heterocycloalkyl, optionally substituted aryl, optionally substituted heteroaryl, —C(O)—OR^{c} and —C(O)—R^{c}, and R^{c} is selected from H, optionally substituted C₁₋₆alkyl, optionally substituted C₃₋₁₀cycloalkyl, optionally substituted heterocycloalkyl, and optionally substituted aryl. Optionally, the resulting carboxylic acid may be converted to a carboxylic ester or lactone (Compound (25)) in a further reaction step taking place in the same one-pot process. For example, in some embodiments, the Method 7 comprises the additional step (D), of esterification or lactonization of the carboxylic acid Compound (24) to form the ester or lactone Compound (25), as shown below:

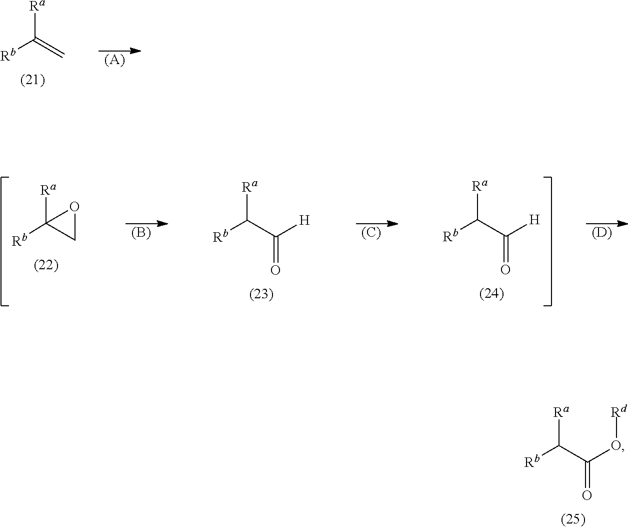

wherein R^{d} is optionally substituted C₁₋₁₂alkyl, or wherein R^{d} and either R^{a} or R^{b} together form a 5-10-membered heterocyclic ring or a C₅₋₁₀carbocyclic ring.

In further embodiments of the seventh aspect, the present disclosure provides:
7.1. Method 7, wherein R^{a} or R^{b} is H;
7.2. Method 7 or 7.1, wherein R^{a} and/or R^{b} are each independently selected from optionally substituted C₁₋₃₀alkyl, optionally substituted C₃₋₁₀cycloalkyl, optionally substituted heterocycloalkyl, optionally substituted aryl, optionally substituted heteroaryl, —C(O)—OR^{c} and —C(O)—R^{c}
7.3. Method 7.2, wherein R^{a} and/or R^{b} are each independently optionally substituted C₁₋₃₀ alkyl, e.g., C₁₋₂₅alkyl, or C₁₋₂₀alkyl, or C₁₋₁₅alkyl, or C₁₋₁₂alkyl, or C₁₋₁₀alkyl, or C₁₋₆alkyl, or C₁₋₃alkyl
7.4. Method 7.3, wherein R^{a} and R^{b} are both independently optionally substituted C₁₋₃₀ alkyl, e.g., C₁₋₂₅alkyl, or C₁₋₂₀alkyl, or C₁₋₁₅alkyl, or C₁₋₁₂alkyl, or C₁₋₁₀alkyl, or C₁₋₆alkyl, or C₁₋₃alkyl
7.5. Method 7.2 or 7.3, wherein R^{a} and/or R^{b} are each independently optionally substituted C₃₋₁₀cycloalkyl, e.g., C₃₋₈cycloalkyl, or C₃₋₆cycloalkyl, or C₃₋₅cycloalkyl, or C₃₋₄cycloalkyl
7.6. Method 7.2, 7.3 or 7.5, wherein R^{a} and/or R^{b} are each independently optionally substituted heterocycloalkyl, e.g., 5-membered or 6-membered heterocycloalkyl, e.g., azetidinyl, aziridinyl, oxetanyl, pyrrolidinyl, tetrahydrofuranyl, piperidinyl, piperazinyl, morpholinyl, thiomorpholinyl, azepanyl, oxazepanyl, and the like;
7.7. Method 7.2, 7.3, 7.5, or 7.6, wherein R^{a} and/or R^{b} are each independently optionally substituted aryl (e.g., phenyl, naphthyl, phenanthryl, and the like)
7.8. Method 7.2, 7.3, 7.5, 7.6, or 7.7, wherein R^{a} and/or R^{b} are each independently optionally substituted heteroaryl (e.g., pyridyl, pyrimidinyl, pyrazinyl, thiophenyl, furyl, pyrrolyl, thiazolyl, oxazolyl, imidazolyl, isothiazolyl, isoxazolyl, pyrazolyl, quinolinyl, quinazolinyl, and the like)
7.9. Method 7.2, 7.3, 7.5, 7.6, 7.7, or 7.8, wherein R^{a} and/or R^{b} are each independently-C(O)—OR^{c} or —C(O)—R^{c};
7.10. Method 7.9, wherein R^{c} is selected from H, optionally substituted C₁₋₆alkyl, optionally substituted C₃₋₁₀cycloalkyl, optionally substituted heterocycloalkyl, and optionally substituted aryl, as those terms are defined in any of embodiments 7.2-7.8 above
7.11. Method 7, or any of 7.1-7.10, wherein the method does not comprise any synthetic steps and/or mechanistic steps other than steps (A), (B), and (C), from Compound (21) through Compound (24)
7.12. Method 7, or any of 7.1-7.11, wherein the method comprises two reaction steps, and wherein in the first reaction step the Compound (21) is treated with a first oxidizing agent in the presence of a suitable solvent (optionally an acid); and wherein in the second reaction step the crude material from the first reaction step is treated with a second oxidizing agent in the presence of a suitable acid and optionally a suitable solvent, followed by isolation of the product Compound (24) from the second reaction mixture; wherein no purification steps are carried out between the two reaction steps (e.g., the reaction mixture from the first reaction step is added directly to the reagents for the second reaction step, or the reagents for the second reaction step are added directly to the reaction mixture from the first reaction step)
7.13. Method 7.12, epoxidation step (A) and rearrangement step (B) take place in the first reaction step, and wherein oxidation step (C) takes place in the second reaction step
7.14. Method 7, or any of 7.1-7.11, wherein the method comprises a single reaction step, wherein in the Compound (21) is treated with an oxidizing agent in the presence of a suitable acid and optionally a suitable solvent; followed by isolation of the product Compound (24) from the reaction mixture
7.15. Method 7.14, epoxidation step (A) and rearrangement step (B) take place in the first reaction step, and wherein oxidation step (C) takes place in the second reaction step
7.16. Any of Methods 7.12-7.15, wherein said oxidizing agent, said first oxidizing agent, and said second oxidizing agent, are each independently selected from hydrogen peroxide, osmium tetroxide, peracetic acid, perchloric acid, perbenzoic acid, meta-chloroperoxybenzoic acid (mCPBA), trifluoroperacetic acid, magnesium monoperoxyphthalate, dimethyl dioxirane (DMDO), tert-butyl hydroperoxide, sodium hypochlorite, sodium tungstate, sodium periodate, iodosyl benzene, pentafluoroiodosyl benzene, cumene hydroperoxide, potassium persulfate, potassium monoperoxysulfate, pyridine N-oxide, 2,6-dichloropyridine N-oxide, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries)
7.17. Method 7.16, wherein said oxidizing agent, said first oxidizing agent, or said second oxidizing agent, is independently selected from hydrogen peroxide, peracetic acid, meta-chloroperoxybenzoic acid, tert-butyl hydroperoxide, or potassium monoperoxysulfate
7.18. Method 7.16, wherein said oxidizing agent is hydrogen peroxide and sodium tungstate, e.g., hydrogen peroxide (e.g., 30 wt. %, e.g., 1-1.5 equivalents) plus sodium tungstate (e.g., sodium tungstate dihydrate, e.g., 0.01-0.10 equivalents) with methyl-tri-n-octylammonium hydrogen sulfate (e.g., 0.01-0.05 equivalents) and phenylphosphonic acid (e.g., 0.01-0.05 equivalents), optionally at 0-50° C., optionally in aqueous solvent
7.19. Method 7.16, wherein said oxidizing agent is m-chloroperoxybenzoic acid
7.20. Method 7.16, wherein said oxidizing agent is oxygen gas
7.21. Method 7.16, wherein said oxidizing agent is oxygen gas with a transition metal catalyst;
7.22. Method 7.16, wherein said oxidizing agent is oxygen gas and iron(III)-tetraphenylporphyrin complex (Fe(III)TPP), in the presence of a C₂-10 aliphatic aldehyde (e.g., isobutyraldehyde)
7.23. Method 7.16, said oxidizing agent is oxygen gas and N-hydroxyphthalimide
7.24. Any of Methods 7.16-7.23, wherein the method does not comprise the use of ozone
7.25. Any of Methods 7.16-7.24, wherein the suitable solvent is independently selected from hydrocarbons (e.g., pentane, hexane, heptane, cyclohexane), chlorinated hydrocarbons (e.g., dichloromethane, chloroform, carbon tetrachloride, 1,2-dichloroethane, tetrachloroethylene), aromatics (e.g., benzene, toluene, xylene, pyridine), ethers (e.g., diethyl ether, diisopropyl ether, methyl tert-butyl ether, tetrahydrofuran, 1,4-dioxane, dimethoxyethane, diethylene glycol dimethyl ether), esters (e.g., methyl acetate, ethyl acetate, isopropyl acetate, butyl acetate), ketones (e.g., acetone, methyl ethyl ketone, methyl isobutyl ketone, cyclohexanone), polar aprotic solvents (e.g., acetonitrile, dimethylformamide, dimethylacetamide, dimethylsulfoxide, hexamethyl phosphoric triamide), polar protic solvents (e.g., water, methanol, ethanol, propanol, isopropanol, butanol, ethylene glycol, glycerol, formic acid, acetic acid, sulfuric acid), acetic anhydride, carbon dioxide (e.g., supercritical carbon dioxide), and carbon disulfide, or a combination thereof
7.26. Any of Methods 7.16-7.25, wherein the reaction comprises the use of 1.0 to 5.0 equivalents of the oxidizing agent, e.g., 1.0 to 4.0 equivalents, or 1.0 to 3.0 equivalents, or 1.0 to 2.0 equivalents, or 1.0 to 1.50 equivalents, or 1.0 to 1.25 equivalents, or 1.0 to 1.15 equivalents, or 1.0 to 1.05 equivalents, or 1.5 to 2.0 equivalents, or 2.0 to 3.0 equivalents; and optionally 0.01 to 1.0 equivalents of any one or more additional reagents (e.g., secondary oxidant, or catalyst, or ligand, or other agent), e.g., 0.01 to 0.5 equivalents, 0.01 to 0.2 equivalents, or 0.01 to 0.1, or 0.01 to 0.05 equivalents
7.27. Any of Methods 7.16-7.26, wherein the acid is a Bronsted acid, e.g., selected from hydrochloric acid, perchloric acid, sulfuric acid, phosphoric acid, acetic acid, trifluoromethanesulfonic acid, methanesulfonic acid, trifluoroacetic acid, and nitric acid, or a heteropoly acid (e.g., phosphotungstic acid);
7.28. Method 7.27, wherein the acid is selected from acetic acid, sulfuric acid, phosphoric acid, and nitric acid
7.29. Any of Methods 7.27-7.28, wherein the reaction is carried out using 1.0 to 5.0 equivalents of the acid, e.g., 1.0 to 4.0 equivalents, or 1.0 to 3.0 equivalents, or 1.0 to 2.0 equivalents, or 1.5 to 4.0 equivalents, or 1.5 to 3.0 equivalents, or 1.5 to 2.5 equivalents, or 1.5 to 2.0 equivalents, or 2.0 to 4.0 equivalents, or 2.0 to 3.0 equivalents
7.30. Any of Methods 7.16-7.29, wherein the reaction step, or the first reaction step and/or the second reaction step, is carried out in a batch reactor
7.31. Any of Methods 7.16-7.29, wherein the reaction step, or the first reaction step and/or the second reaction step, is carried out in a continuous flow reactor
7.32. Any of Methods 7.16-7.31, wherein the solvent is acetic acid and/or acetic anhydride, and optionally wherein the reaction further comprises an acetate salt (e.g., sodium or potassium acetate)
7.33. Any of Methods 7.16-7.31, wherein the reaction step, or the first reaction step and/or the second reaction step, is carried out using a combination of hydrogen peroxide, sulfuric acid, and acetic acid, optionally 1.5-4 equivalents of each (e.g., 2-3 equivalents of each)
7.34. Any of Methods 7.16-7.31, wherein the reaction step, or the first reaction step and/or the second reaction step, is carried out using a combination of acetic acid, peracetic acid, and sulfuric acid, optionally 1-3 equivalents of each (e.g., 1-1.5 equivalents of each)
7.35. Any of Methods 7.16-7.31, wherein the reaction step, or the first reaction step and/or the second reaction step, is carried out using a combination of hydrogen peroxide, acetic acid, peracetic acid, and sulfuric acid, optionally 0.8-5 equivalents of each (e.g., 1-3 equivalents of each), optionally with sodium acetate (e.g., 0.05-0.24 equivalents)
7.36. Any of Methods 7.16-7.31, wherein the reaction step, or the first reaction step and/or the second reaction step, is carried out using a combination of hydrogen peroxide, acetic anhydride, acetic acid, and sulfuric acid, optionally 0.8-5 equivalents of each (e.g., 1-3 equivalents of each), optionally with sodium acetate (e.g., 0.05-0.24 equivalents), further optionally with heptane co-solvent
7.37. Any of Methods 7.16-7.31, wherein the reaction step, or the first reaction step and/or the second reaction step, is carried out using a combination of potassium monoperoxysulfate, potassium hydrogen sulfate and potassium sulfate (e.g., Oxone®), in organic solvent (e.g., acetone/ethyl acetate), optionally 1-3 equivalents of potassium monoperoxysulfate (e.g., 1-2 equivalents)
7.38. Any of Methods 7.16-7.31, wherein the reaction step, or the first reaction step and/or the second reaction step, is independently carried out at a temperature of 0° C. to 200° C., e.g., 0° C. to 150° C., or 0° C. to 100° C., or 25° C. to 200° C., or 25° C. to 150° C., or 25° C. to 100° C., or 50° C. to 200° C., or 50° C. to 150° C., or 50° C. to 100° C., or 75° C. to 200° C., or 75° C. to 150° C., or 75° C. to 100° C., or 80° C. to 150° C., or 80° C. to 100° C., or 90° C. to 150° C., or 90° C. to 125° C., or 90° C. to 110° C., or 90° C. to 100° C., or 90° C. to 95° C.
7.39. Any of Methods 7.16-7.31, wherein the reaction step, or the first reaction step and/or the second reaction step, is independently carried out at a temperature of 80° C. to 90° C., 20° C. to 70° C., 40° C. to 90° C., 20° C. to 50° C., or 20° C. to 30° C.
7.40. Method 7, or any of Methods 7.1-7.32, wherein reactant Compound (21) proceeds to the product Compound (24) in a single vessel
7.41. Method 7, or any of 7.1-7.40, wherein the method does not comprise the use of any reagents or reactants other than the Compound (21), and the reagents set forth herein (e.g., acids, oxidizing agents, solvents), for example, the method does not comprise the use of any carbon monoxide, carbonyl equivalents, enzymes, ozone, or cyanide reagents
7.42. Method 7, or any of 7.1-7.41, wherein the method further comprises the step (D) of esterification or lactonization of the Compound (24) to form an ester or lactone Compound (25), e.g., a C₁₋₁₂alkyl ester or a monocyclic or bicyclic lactone, such as a methyl, ethyl, propyl, isopropyl, n-butyl, t-butyl, isobutyl, sec-butyl, n-pentyl, t-amyl, n-hexyl, n-heptyl, n-octyl, n-nonyl, n-decyl, or isodecyl ester.

It is understood that the Method 7 is generic to the more specific Methods 1, 2, and 3, because the compounds (5) and (10) are species of the generic compound (24), and compounds (2) and (7) are species of the generic compound (21), and the different methods proceed through the same mechanistic steps (A), (B), and (C), although Methods 1, 2, and 3 comprise the further required mechanistic step (D). The Method 7 includes the corresponding step (D) as an optional step, which would result in the Compound (25) which corresponds to and is generic to the Compounds (1) and (6).

In an eighth aspect, the present disclosure provides Compound (24) or Compound (25) made according to Method 7 or any of 7.1-7.42.

In a ninth aspect, the present disclosure provides a composition comprising Compound (24) or Compound (25), made according to Method 7 or any of 7.1-7.42, wherein said compound imparts a flavor or fragrance to the composition. The further embodiments described hereinabove for the sixth aspect also apply to this ninth aspect.

In a tenth aspect, the present disclosure provides a product or composition, such as an organoleptic composition, comprising Compound (1), made according to Method 1 or any of 1.1 et seq. or Method 3 or any of 3.1 et seq., or Compound (6), made according to Method 2 or any of 2.1 et seq. or Method 3 or any of 3.1 et seq., or Compound (24) or Compound (25), made according to Method 7 or any of 7.1-7.42. In some embodiments, the Compound (1) or Compound (6) or Compound (24) or Compound (25) may be used alone as a fragrance or added into a fragrance composition and/or consumer product as an agent for increasing substantivity and/or retention of a fragrance preparation and/or as a fixative.

Suitable solvents for such compositions may include, alcohols such as methanol, ethanol, propanol, isopropanol, butanol, tert-butanol and the like; lower alkyl ester of lower carboxylic acid such ethyl acetate and the like; alkane nitriles such as acetonitrile, propionitrile, butyronitrile, isobutyronitrile, valeronitrile and the like; aromatic hydrocarbons such as benzene, toluene, xylene, anisole and the like; aliphatic hydrocarbons such as pentane, hexane, heptane, octane, cyclohexane, cycloheptane, cyclooctane and the like; and water. All these solvents can be used singly or in mixture with each other. Water can also be used as a solvent with or without mixing above mentioned solvents during the reaction.

The present disclosure further provides for the use of a compound of Formula XIII, XIV or XV, XIIIa, XIIIb, XIVa, XIVb, and/or XVa, as a flavor or fragrance agent, e.g., in order to impart a flavor or fragrance to a composition or product. The present disclosure further provides for the use of a Compound (1), made according to Method 1 or any of 1.1 et seq. or Method 3 or any of 3.1 et seq., or Compound (6), made according to Method 2 or any of 2.1 et seq. or Method 3 or any of 3.1 et seq., or Compound (24) or Compound (25), made according to Method 7 or any of 7.1-7.42, as a flavor or fragrance agent, e.g., in order to impart a flavor or fragrance to a composition or product.

As used herein, the term “aqueous solvent” refers to a solvent mixture having any amount of water, including trace water (for example, where the solvent is one that is miscible with water). The water for the “aqueous solvent” may also be provided by aqueous reagents (such as aqueous acids, e.g., sulfuric acid, or aqueous oxidizing agents, such as hydrogen peroxide). Without being bound by theory, it is believed that in some embodiments, water (or another protic solvent) may only be necessary in a catalytic amount for the methods described herein to proceed, so significant amounts of water need not be used in the reactions. In some embodiments, the reactions may proceed in an autocatalytic fashion.

It is understood that hydrogen peroxide and peracetic acid (also known as peroxyacetic acid) are both commonly available as solutions, not as pure compounds. Hydrogen peroxide is sold as an aqueous solution consisting essentially of 10-70% hydrogen peroxide and the balance water, but with small amounts of other ingredients (e.g., <5% of stabilizing agents or impurities). Peracetic acid is commercially made by oxidizing dilute acetic acid with hydrogen peroxide. As a result, peracetic acid is commonly sold as a solution of 15% or 32% peracetic acid in an aqueous acetic acid carrier. The solution will commonly have small amounts of unreacted hydrogen peroxide, as well as possibly stabilizing agents or other impurities.

As used herein, the term “fragrance composition” means a mixture of fragrance ingredients, including auxiliary substances if desired, dissolved in a suitable solvent or mixed with a powdery substrate used to provide a desired odor to a product. Examples of products having fragrance compositions include, but are not limited to, perfumes, soaps, insect repellants and insecticides, detergents, household cleaning agents, air fresheners, room sprays, pomanders, candles, cosmetics, toilet waters, pre- and aftershave lotions, talcum powders, hair-care products, body deodorants, anti-perspirants, and pet litter.

As used herein, the term “flavor composition” means a mixture of flavor ingredients, including auxiliary substances if desired, dissolved in a suitable solvent or mixed with a powdery substrate used to provide a desired flavor to a product. Examples of products having flavor compositions include, but are not limited to, dental hygiene products such as mouth wash, toothpaste, floss, and breath fresheners, orally administered medications including liquids, tablets or capsules, and food products.

Fragrance and flavor ingredients and mixtures of fragrance and flavor ingredients that may be used in combination with the compound of Formula XIII, XIV or XV, XIIIa, XIIIb, XIVa, XIVb, and/or XVa, or Compound (1), or Compound (6), or Compound (24) or Compound (25), for the manufacture of fragrance and flavor compositions include, but are not limited to, natural products including extracts, animal products and essential oils, absolutes, resinoids, resins, and concretes, and synthetic fragrance materials which include, but are not limited to, alcohols, aldehydes, ketones, ethers, acids, esters, acetals, phenols, ethers, lactones, furansketals, nitriles, acids, and hydrocarbons, including both saturated and unsaturated compounds and aliphatic carbocyclic and heterocyclic compounds, and animal products.

As used herein, “optionally substituted” means that the indicated functional group is either unsubstituted or substituted by one or more groups up to the maximum permitted by the rules of valency, wherein said groups are selected from: halo, hydroxy, cyano, C₁₋₆alkyl, C₂-6alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), aryl, and heteroaryl; wherein each of said C₁₋₆alkyl, C₃₋₆cycloalkyl, aryl or heteroaryl is further optionally substituted by one or more halo, hydroxy, cyano, C₁₋₆alkyl, C₂₋₆alkenyl, C₂₋₆alkynyl, C₃₋₆cycloalkyl, C₁₋₆haloalkyl, —O—Si(R^{x})₃, —O—R^{x}, —C(O)H, —C(O)—R^{x}, —C(O)—O—R^{x}, —C(O)—NH—R^{x}, —C(O)—N—(R^{x})(R^{x}), —O—C(O)—R^{x}, —NH(R^{x})—C(O)—R^{x}, —N(R^{x})(R^{x})—C(O)—R^{x}), —NH(R^{x}), —N(R^{x})(R^{x}), aryl, and heteroaryl; and wherein each R^{x} is independently selected from hydrogen, C₁₋₆alkyl, C₂₋₆alkenyl, C₂₋₆alkynyl, C₃₋₆cycloalkyl, aryl and heteroaryl. Aryl includes, but is not limited to, optionally substituted phenyl, and optionally substituted naphthyl. Heteroaryl includes, but is not limited to, any optionally substituted furan, thiophene, pyrrole, pyrazole, oxazole, isoxazole, thiazole, isothiazole, pyridine, pyrimidine, pyridazine, pyrazine, indole, benzofuran, benzoxazole, benzisoxazole, benzothiazole, quinoline, isoquinoline, quinazoline, and quinoxaline.

In Method 1 et seq. and Method 2 et seq., epoxidation step (A) may be carried out using any suitable set of reaction conditions known in the art. Epoxidation is often carried out using a strong oxidizing agent, such as mCPBA, or a combination of oxygen with a catalyst, or using a combination of oxidizing agents of differing strengths. Commonly used reagents include one or more of hydrogen peroxide, osmium tetroxide, peracetic acid, perchloric acid, perbenzoic acid, meta-chloroperoxybenzoic acid, trifluoroperacetic acid, magnesium monoperoxyphthalate, dimethyl dioxirane (DMDO), tert-butyl hydroperoxide, sodium hypochlorite, sodium tungstate, or oxygen (e.g., in combination with a transitional metal catalyst, e.g., an iron catalyst). In some embodiments, transition metal-doped silica or zeolite catalysts may be used with oxygen and a secondary oxidant, such as tert-butyl hydroperoxide, or with a co-reagent, such as an aliphatic aldehyde. For example, epoxidation can be carried out according to the procedure of Madadi et al., Applied Cat. B: Environ. 260 (2020) 118049, using the catalyst mesoporous SBA-16 silica modified with cobalt, titanium, nickel, iron, or manganese, and reacted with the alkene substrate in the presence of oxygen and a C₂₋₁₀ aliphatic aldehyde (e.g., isobutyraldehyde). In some embodiments, oxygen or peroxides (e.g., hydrogen peroxide) can be used as the oxidant in combination with a C₂₋₁₀ aliphatic aldehyde (e.g., isobutyraldehyde), and a transition metal porphyrin complex (e.g., tetraphenyl porphyrin), as described in Chinese patent publication CN1915983A (“Method for preparing epoxy compound by oxidizing olefin or cycloolefin through bionic catalytic oxygen”). Suitable transition metals for the catalyst include manganese, iron, or ruthenium, and the phenyl groups of the porphyrin ring may be unsubstituted or para- and/or ortho-substituted with electron withdrawing groups (e.g., nitro, fluoro, chloro). Other suitable transition metals include titanium, chromium, molybdenum, osmium, and cobalt, while other suitable oxidants include iodosyl benzene, pentafluoroiodosyl benzene, mCPBA, sodium hypochlorite, tert-butyl hydroperoxide, cumene hydroperoxide, potassium persulfate, pyridine N-oxide, and 2,6-dichloropyridine N-oxide. See Amal Salmeen Basaleh, “The Kinetics and Mechanism of the Activation of Metalloporphyrin by Hydrogen Peroxide,” Dissertation (Univ. of Surrey, Guildford, U. K., & King Abdulaziz Univ., Jedda, Saudi Arabia) (July 2013). A particularly effective epoxidation method employs hydrogen peroxide with catalytic sodium tungstate, and optionally various additives, such as sodium sulfate, methyl-tri-n-octylammonium hydrogen sulfate, and/or phenylphosphonic acid (Noyori oxidation), such as reported by Hachiya et al., Syn. Lett. 19:2819-22 (2011). A simple mCPBA-mediated epoxidation of isopulegol is reported by Zhao et al., Tet. Lett. 45(19):2713-16 (2004).

In some embodiments wherein R is H, use can be made of the hydroxy group for a directed (e.g., asymmetric) epoxidation under mild conditions. For example, as described in Gill et al., Chem. Commun. 1743-1744 (1996), the epoxidation may be carried out using vanadyl acetylacetonate (VO(acac)₂), in the presence of tert-butyl hydroperoxide in benzene solvent (or toluene). Guidotti et al., Chem. Commun. 1789-1790 (2000) similarly report the epoxidation of isopulegol using mesoporous titanium MCM-41 complex with tert-butyl hydroperoxide. The use of the simple oxidation system Oxone/acetone/sodium bicarbonate has been reported by Ferraz et al, Tet. Lett. 41(26):5021-23 (2000).

In Method 1 et seq. and Method 2 et seq., rearrangement step (B) may be carried out using any suitable set of reaction conditions known in the art, such as the Meinwald Rearrangement. In some embodiments, rearrangement may be carried out thermally without the addition of a reagent. In some embodiments, the rearrangement may be catalyzed by acid, e.g., by a Bronsted acid, a Lewis acid, or a combination thereof. In some embodiments, the reaction may be catalyzed by strong base, such as an alkyl lithium or lithium amide base (e.g., sec-butyl lithium, tert-butyl lithium, or lithium 2,2,6,6-tetramethylpiperide (LiTMP), lithium diisopropylamide (LDA), lithium bis(trimethylsilyl)amide) (LiHMDS)). See, e.g., Kumar & Jat, Adv. Synth. Catal., DOI 10.1002/adsc.201900392 (2019) (disclosing conversion of epoxides to aldehydes using 1.5 eq. LiTMP in THF at room temperature for 12 hours). In some embodiments, the reaction may be catalyzed by transition metal complex (e.g., palladium, ruthenium, rhodium, chromium, iridium, manganese, iron, or nickel catalyst or complex). Kumar & Jat also disclose palladium(0) catalyzed epoxide rearrangement, such as using Pd(0)-tertiary phosphine complexes (e.g., palladium acetate plus triphenylphosphine or tributyl phosphine), in solvents, such as benzene, toluene, and tert-butanol, as well as rearrangements catalyzed by palladium hydride complexes and iridium hydride complexes. Kumar & Jat also disclose regioselective and stereoselective rearrangement using transition metal tetraphenyl porphyrin complexes as Lewis acids, such as chromium (III) tetraphenyl porphyrin triflate (e.g., in chlorinated solvent such as dichloroethane). Other transition metals, such as iron, ruthenium, rhodium, or manganese, and modified porphyrin structures, such as tetratolyl porphyrin or tetra(2,4,6-trimethylphenyl) porphyrin complexes, can also be used (e.g., dichloro ruthenium tetra(2,4,6-trimethylphenyl) porphyrin complex). Other methods disclosed in Kumar & Jat use nickel bis(triphenylphosphine) dibromide complex (e.g., in THF solvent at elevated temperature), or methyl bis(4-bromo-1,6-di-tert-butylphenoxy) aluminum (e.g., in dichloromethane solvent at room temperature). Humbert et al., Chem. Comm. 50(73):10392-95 (2014) report epoxide rearrangement catalyzed by palladium hydride, ruthenium hydride, zirconium hydride, and iridium hydride catalysts, e.g., [(1,3-bis(di-isopropylphosphino)propane)₃Pd₂H₂] bistriflate, [(Ph₃P)₃RuH(Cl)] (optionally with a bisphosphine ligand, e.g., sodium tetrakis-[(3,5bis(trifluoromethyl)phenyl)borate]), Schwartz reagent ([(cyclopentadienyl)₂Zr(H)Cl]), or the unique cyclometallated iridium(III) hydride complex disclosed by Humbert (Ir(III) complexed with 1,5-cyclooctadiene, tricyclohexylphosphine, and 8-methylquinoline). Additional methods for epoxide rearrangement are disclosed in Arata & Tanabe, Catal. Rev. Sci. Eng. 25(3):365-320 (1983) (e.g., thermal rearrangement, rearrangement catalyzed by 1% sulfuric acid in glacial acetic acid, p-toluenesulfonic acid, magnesium bromide diethyl etherate, zinc bromide, zinc chloride, lithium bromide, boron trifluoride, borane). Takamani et al., Chem. Lett. 1031-32 (1996) reports the use of iron (III) tetraphenylporphyrin perchlorate complex (e.g., in dichloromethane solvent). Another potential acidic catalyst for this rearrangement is the heteropolyacids, such as phosphotungstic acid (H₃PW₁₂O₄₀), such as similarly described in Gusevskaya et al., Chem. Eur. J. 14:6166-72 (2008). Stork et al., JAGS 118(43):10660-61, report a typical set of conditions for epoxidation of an exocyclic double bond using mCPBA followed by rearrangement of the epoxide to the aldehyde catalyzed by boron trifluoride etherate complex.

In similar work, Jiang et al., Angew. Chem. 120:6740-44 (2008) report a combined method of epoxidizing an alkene and rearranging the epoxide to the aldehyde in-situ using a ruthenium porphyrin complex with a terminal oxidant, such as air 2,3-dichloropyridine N-oxide. The intermediate epoxide is not isolated and the aldehyde results in good yields. Catalysts include dichloro ruthenium tetra(2,4,6-trimethylphenyl)porphyrin, dichloro ruthenium tetra(2,6-dichlorphenyl)porphyrin, dioxo ruthenium tetra(2,4,6-trimethylphenyl)porphyrin, dioxo ruthenium tetra(2,6-dichlorphenyl)porphyrin, and dioxo ruthenium tetramethyl-tetra(2,6-diphenylphenyl)porphyrin.

In Method 1 et seq. and Method 2 et seq., oxidation step (C) may be carried out using any suitable set of reaction conditions known in the art. Typically, an aldehyde to carboxylic acid oxidation is carried out using a strong oxidizing agent, such as a chromium oxidant (e.g., chromium trioxide, chromic acid, pyridinium chlorochromate, potassium dichromate, chromium trioxide-pyridine complex, pyridinium dichromate), osmium tetroxide, potassium permanganate, silver oxide, hydrogen peroxide, peracetic acid, perchloric acid, trifluoroperacetic acid, periodic acid, potassium periodate, sodium chlorite, oxygen and N-hydroxyphthalimide, or potassium monoperoxysulfate. Jones Reagent (chromium trioxide in aqueous sulfuric acid) is a particularly effective oxidizing system. Chinese patent application CN108315499A also discloses an environmentally friendly oxidation method using an oxygen atmosphere with an N-hydroxyimide catalyst, such as N-hydroxyphthalimide, N-hydroxymaleimide, N-hydroxysuccinimide, N-hydroxyglutarimide N-hydroxy-1,8-napthalimide, or N-hydroxy-benzene dicarboximide (e.g., 0.05-0.15 equivalents, in a suitable solvent, e.g., acetonitrile), optionally in conjunction with a nitrite agent (e.g., methyl nitrite, ethyl nitrite, propyl nitrite, isopropyl nitrite, butyl nitrite, isoamyl nitrite, tert-butyl nitrite, or benzyl nitrite).

Other suitable transition metal catalyst complexes are described in Piccirilli et al., Catalysts 20:773 (2020).

In Method 3 et seq., and Method 7 et seq., it is believed that the method may be carried out using any oxidizing agent capable of both epoxidation and aldehyde oxidation, as described in the preceding paragraphs.

It is further understood that the oxidizing agent potassium monoperoxysulfate (also known as “MPS”, having the formula KHSO₅) is commonly sold under the trade names Oxone® and Caroat®, which are triple salt mixtures consisting of about 2 parts KHSO₅, 1 part KHSO₄, and 1 part K₂SO₄, and being approximately 47-50% by weight of KHSO₅. The active oxygen species KHSO₅ is more stable in this mixture than in pure form.

### EXAMPLES

NMR spectra are recorded using a 500 MHz NMR spectrometer. All ¹H-NMR data are reported in δ units, parts per million (ppm), and are calibrated relative to the signals for residual chloroform (7.26 ppm) in deuterochloroform (CDCl₃). All ¹³C-NMR data are reported in ppm relative to CDCl₃ (77.16 ppm) and are obtained with 1H decoupling. The following abbreviations or combinations thereof were used to explain the multiplicities: s=singlet, d=doublet, t=triplet, q=quartet, br=broad, m=multiplet, a=apparent.

GC Analysis is performed on an Agilent 6890N gas chromatograph with a Restek-Stabilwax (crossbond Carbowax Polyethylene glycol) 30 mm×0.25 mm×0.25 μm column (cat. #10623). Injection volume is 1 μL (splitless). Injection temperature is 250° C. Maximum oven temperature is 220° C., with an initial setpoint temperature of 100° C. and ramp rate of 20° C./min. Carrier gas is helium. Flow rate is 1.8 mL/min. Pressure is 22.39 psi. A flame ionization detector at 220° C. is used.

HPLC is performed using an Agilent HPLC 1100 series with UV detector.

### Example 1: (1R,2R,5R)-5-methyl-2-(2-methyloxiran-2-yl)cyclohexan-1-ol

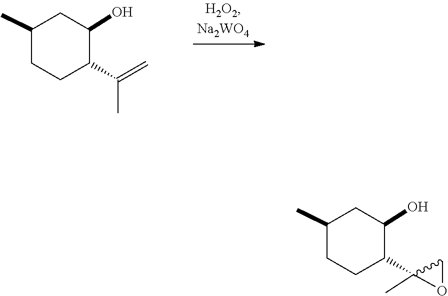

In a 100 ml flask was charged (−)-isopulegol ((1R,2S,5R)-5-methyl-2-(prop-1-en-2-yl)cyclohexan-1-ol) (10 g, 0.065 mol), sodium tungstate dihydrate (1.28 g, 0.06 eq.), methyl-tri-n-octylammonium hydrogen sulfate (1.51 g, 0.003 mol, 0.05 eq.) (Me(n-octyl)₃NHSO₄), and phenylphosphonic acid (0.31 g, 0.0019 mol, 0.03 eq.) (PhPO₃H₂). Hydrogen peroxide (30% aqueous, 8.1 ml. 0.071 mol, 1.1 eq.) is added dropwise over 0.5 hour, and then the reaction mixture is stirred at room temperature overnight (20 hours). The reaction mixture is diluted with MTBE (40 ml) and washed with water, brine, dried with Na₂SO₄, and purified by silica gel flash chromatography (EtOAc/hexanes: 20 to 40%) to afford the product (9.5 g, 86.2%) as a 1:1 isomeric mixture ((1R,2R,5R)-5-methyl-2-((S)-2-methyloxiran-2-yl)cyclohexan-1-ol and (1R,2R,5R)-5-methyl-2-((R)-2-methyloxiran-2-yl)cyclohexan-1-ol).

### Example 2: (3aS,6R,7aR)-3,6-dimethylhexahydrobenzofuran-2(3H)-one

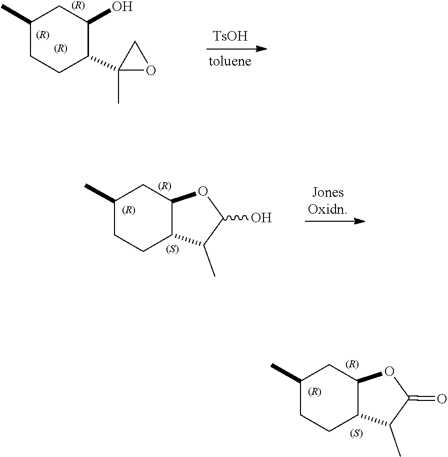

To a solution of the product of Example 1 (2.0 g, 0.01118 mol) in toluene (6 ml) is added Montmorillonite K10 (0.2 g). The mixture is heated at 75° C. for 30 minutes and then cooled in ice-water bath. Acetone (3 ml) is added, then Jones Reagent (3M chromium trioxide in aqueous sulfuric acid) is added slowly over 10 minutes (3M, 4.7 ml, 0.0147 mol). The mixture is stirred at room temperature for 0.5 hours and is then quenched by the addition of 2 ml of isopropanol and stirred at room temperature for an additional 0.5 hour. The mixture is then diluted with 15 ml of EtOAc and the phases are separated. GC analysis shows the product forms at an isomer ratio 68.6 to 14 and a crude conversion yield of 82.6% (net, both isomers). The organic layer is washed with water (2×10 ml), dried (Na₂SO₄), and purified by silica gel flash chromatography (EtOAc/hexanes: 0 to 5%) to afford 0.5 g of the product (25% yield) as a mixture of the two isomers, (3S,3aS,6R,7aR)-3,6-dimethylhexahydrobenzofuran-2(3H)-one and (3R,3aS,6R,7aR)-3,6-dimethylhexahydrobenzofuran-2(3H)-one at an 83:17 ratio.

### Example 3: 3,6-dimethylhexahydrobenzofuran-2(3H)-one via H₂SO₄/H₂O₂/HOAc

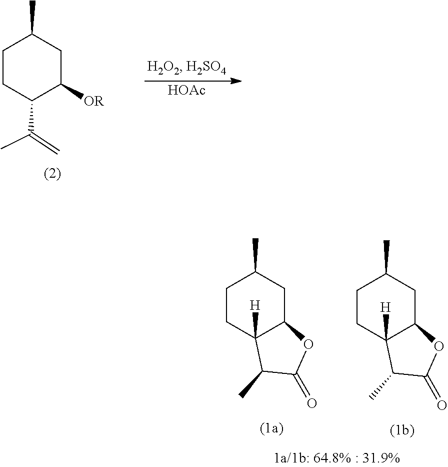

To a 500 ml 4-neck flask, equipped with a mechanical stirrer, is charged acetic acid 15 ml (0.26 mol, 2 eq.) and the flask is preheated to 90° C. Sulfuric acid (60% aqueous, 42 g, 0.26 mol, 2 eq.) and hydrogen peroxide (30% aqueous, 44.2 g, 3 eq.) are added via two separate dropping funnels. At the same time, isopulegol (20 g, 0.13 mol) is added by syringe pump at a rate of 40 ml/hr. The temperature is controlled at 90-95° C. during the addition process. The reaction mixture is further stirred at 95° C. for 2 hours, then it is cooled down in cold water and diluted with 100 ml of ice water. The mixture is extracted with EtOAc (3×50 ml). The combined EtOAc solutions are washed with water (30 ml), washed with saturated sodium carbonate (2×50 ml), washed with 10% thiodiglycol solution (30 ml), washed with brine (30 ml), and then dried over sodium sulfate, and concentrated to dryness under vacuum to obtain an oil. The oil is dissolved in 50 ml hexanes and passed through a plug of silica gel (50 g, 40 mm column) and eluted with EtOAc/hexane (8% to 12%). The eluate is concentrated to dryness to afford the crude product (13.5 g, 62% yield). GC analysis indicates a purity of 95.7%, and an isomeric ratio of isomer (1a) 64.8%; isomer (1b) 30.9% (isomer (1a) appears at about RT 11.87; isomer (1b) appears at about RT 12.49).

### Example 4: Isomerization of 3,6-dimethylhexahydrobenzofuran-2(3H)-one

A crude mixture of 3,6-dimethylhexahydrobenzofuran-2(3H)-one obtained from a procedure analogous to Example 3, and having an isomeric ratio of 65:34 isomer (1a): isomer (1b), is dissolved in 70 mL heptane and the mixture is refluxed with a Dean-Stark apparatus to remove water until about 50 mL of heptane is collected. The reaction mixture is then cooled down to room temperature, and sodium tert-butoxide (0.2 g) is added. The mixture is stirred at room temperature for 18 hours and then another portion of sodium tert-butoxide (0.2 g) is added. The mixture is further stirred for 22 hours, and then it is diluted with water (30 ml) and extracted with methyl tert-butyl ether (MTBE) (30 ml). The MTBE solution is washed with brine (20 ml), dried over sodium sulfate, and concentrated to dryness to afford 7.0 g oil. GC analysis shows that the isomeric ratio is 93.6% isomer (1a) to 6.4% isomer (1b).

### Example 5: Crystallization of 3,6-dimethylhexahydrobenzofuran-2(3H)-one

A crude mixture of 3,6-dimethylhexahydrobenzofuran-2(3H)-one (52 g), containing 90.8% of product with the isomers (1a) and (1b) in a 94.8:5.2 ratio, is dissolved in 209 g n-hexane with stirring over ten minutes. The mixture is then placed in a freezer at −10° C. for 15 hours. A white crystalline product is observed to have formed. The crystals are separate by filtration and washed with 10 mL of cold n-hexane. The crystals are then transferred to a 50-mL round bottom flask and heated neat in an oil bath at 50° C. The resulting liquid is dried under high vacuum (about 5 Torr) at 50° C. for 2 hours with stirring. The liquid is then transferred to a flat surface, where it solidifies. The solid is broken up to form flakes. 29 g of solid flakes are collected. GC analysis shows the major isomer present at 98.6% (1a).

### Example 6: 3,6-dimethylhexahydrobenzofuran-2(3H)-one Via Peracetic Acid (One-Pot Procedure)

To 2 g (0.013 mol) of isopulegol is added 32% peracetic acid dropwise (−3 ml from a total of 9.3 g, 0.039 mol, 3 eq.) over a period of 10 minutes at room temperature. An exothermic phenomenon is observed during the process. The reaction mixture is then stirred at ambient temperature for 1 hour. A mixture of triflic acid in acetic acid (1 g TfOH, 0.5 eq.; 2 mL HOAc, 2.7 eq.) is added dropwise. The reaction mixture is stirred at ambient temperature for 2 hours. GC analysis shows that the desired lactone is formed, and the major isomer content is 48.0%, and the minor isomer content is 24.8%. The total product yield by GC is 73%. The reaction may also be performed by combining peracetic acid, triflic acid, and acetic acid at the outset.

### Example 7: 3,6-dimethylhexahydrobenzofuran-2(3H)-one Via Peracetic Acid (Alternative One-Pot Procedure)

To a 500 ml 4-neck flask is charged 15 mL of acetic acid (0.26 mol) and the flask is preheated to 80° C. Sulfuric acid (60% aq., 64 g, 0.26 mol) and 15% peracetic acid solution (63.6 g, 0.125 mol) (15% peracetic acid, 22% hydrogen peroxide, 16% acetic acid, 46% water; total active oxygen 13.7%) are added via two separate dropping funnels. At the same time, isopulegol (30 g, 0.195 mol) is added by syringe pump at a rate of 40 ml/hr. The temperature is controlled at 80-85° C. during the addition process. The reaction mixture is further stirred at 80° C. for 1.5 hours, then it is cooled down in cold water and diluted with 60 ml of ice water. The mixture is extracted with hexanes (3×60 ml). The combined hexane solution is washed with water (30 ml), 20% NaHSO₃ (30 ml), saturated sodium carbonate (100 ml), brine (30 ml), and then the crude solution is passed through a plug of silica gel, and eluted with EtOAc/hexane (8% to 15%). The eluate is concentrated to dryness and dissolved in 80 ml of hexanes. The hexane solution is isomerized with sodium tert-butoxide (0.6 g) and crystalized as described in Example 4 and 5 to afford the product (7.8 g, 24%).

### Example 8: 3,6-dimethylhexahydrobenzofuran-2(3H)-one Via Peracetic Acid (Two-Step Procedure)

To a 1 L 3-neck flask is charged isopulegol (100 g, 1.297 mol) and sodium acetate (8 g, 0.097 mol) and the mixture is cooled in ice-water bath. 32% Peracetic acid solution (216 g, 0.908 mol) (32% peracetic acid, 40-50% acetic acid, <8% hydrogen peroxide, balance water) is added dropwise over 40 minutes and then the reaction is further stirred in a water bath at about 20° C. for 1 hour.

In a 1-L 3-neck flask is charged 60% sulfuric acid (140 ml) and this is preheated to 60° C. Then the mixture from the first reaction step is added via syringe pump at 5 ml/min. At the same time, hydrogen peroxide (30% aqueous, 88 g) is added via syringe pump at 0.6 ml/min. The temperature is controlled at <62° C. during the additions. The reaction mixture is then further stirred at 60° C. for 1 hour after the hydrogen peroxide addition is finished. The reaction mixture is cooled down, diluted with water (100 ml), and extracted with heptane (200 ml, 2×150 ml). The combined heptane solutions are washed with water (100 ml), 10% Na₂SO₃, 2N NaOH (50 ml), brine (2×30 ml), and then dried (Na₂SO₄), and distilled under vacuum to afford the product (61.3 g, GC purity major isomer (1a) 60.1%, minor isomer (1b) 22.5%; GC yield 46%).

### Example 9: 3,6-dimethylhexahydrobenzofuran-2(3H)-one Via Hydrogen Peroxide (Two-Step Procedure)

To a 1 L 3-neck flask is charged isopulegol (200 g, 1.297 mol) and sodium acetate (14 g, 0.169 mol). The flask is preheated to 40° C. and then hydrogen peroxide (30% aqueous, 206 g, 1.816 mol) and acetic anhydride (211 g, 2.08 mol) are added separately via syringe pump at 4 ml/min. The temperature is controlled at 40-60° C. during the addition. After the addition is finished, the mixture is stirred at 45° C. for 1.5 hours, then the reaction mixture is cooled to about 15° C., and additional hydrogen peroxide is added (30% aqueous, 206 g, 1.816 mol).

In a 2-L Syrris Reactor is charged 100 g of acetic acid and 100 ml of sulfuric acid (60% aqueous, from total 530 g, 3.24 mol) and the mixture is preheated to 75° C. The reaction mixture from the first reaction step and the remaining sulfuric acid are added separately by dropping funnel over 85 minutes. The temperature is controlled at 80-88° C. during the additions. The reaction mixture is further stirred at 80° C. for 1.5 hours, then it is cooled down to below 20° C. The reaction mixture is diluted with water (400 ml), and extracted with heptane (500 ml, 2×250 ml). The combined heptane solutions are washed with water, 20% NaHSO₃, 2N NaOH, brine, and then dried (Na₂SO₄), and the crude solution is passed through a plug of silica gel and eluted with EtOAc/hexane (8% to 15%). The collected eluate is concentrated to dryness and dissolved in 650 ml of hexanes. To the hexane solution is added sodium tert-butoxide (5 g). The mixture is stirred overnight, and then it is quenched by addition of water (50 ml). The phases are separated, and the organic layer is washed with 0.5 N NaOH, brine, and dried (Na₂SO₄). recrystallized at −20° C., as described in Example 5. The obtained crystals are washed with cold hexane to afford the product (81 g, 37%).

### Example 10: 3,6-dimethylhexahydrobenzofuran-2(3H)-one Via Hydrogen Peroxide (Two-Step Procedure with Co-Solvent)

To a 500 ml 3-neck flask is charged isopulegol (50 g, 0.324 mol) and sodium acetate (1.25 g). The flask is preheated to 40° C. in a water bath, and then hydrogen peroxide (30% aqueous, 50 ml from a total of 95.2 g, 0.843 mol) and acetic anhydride (52.9 g, 0.519 mol) are added separately via dropping funnels over 40 minutes. The temperature is controlled at 40-60° C. during the additions. The reaction is stirred in the water bath for 1 hour. The reaction mixture is then cooled to below 20° C. and the remaining hydrogen peroxide is added.

In a 1000 mL 3-neck flask is charged 10 ml acetic acid and 10 ml of sulfuric acid (60% aqueous, from a total of 106 g, 0.65 mol) and heptane (80 ml). The mixture is heated in a 90° C. heating bath and the reaction mixture from the first reaction step, and the rest of the sulfuric acid, are added separately over 50 minutes. The reaction mixture is gently refluxed during the process. The reaction mixture is further stirred for 1 hour after the additions. The reaction mixture is then was cooled down, diluted with water (100 ml), the phases are separated, and the aqueous layer is extracted with heptane (2×50). The combined heptane solutions are washed with water, 20% NaHSO₃, 2N NaOH, brine, dried (Na₂SO₄), and then the crude solution is passed through a plug of silica gel and eluted with EtOAc/hexane (8% to 15%). The eluate is concentrated to dryness. The crude product is dissolved in 250 ml of hexanes, isomerized using sodium tert-butoxide, and recrystallization from hexane, as described in Example 4 and 5, to afford the product (21.2 g, 39%).

### Example 11: 3,6-dimethylhexahydrobenzofuran-2(3H)-one Via Oxone

To a 2 L three-necked flask are added (−) isopulegol (26.8 g, 0.174 mol), acetone (160 mL, 2.160 mol) and ethyl acetate (270 mL), and the mixture is stirred vigorously. An aqueous solution of Oxone® (121.0 g, 0.398 mol; in water 500 mL) is added dropwise over 1 hour maintaining the temperature at 20 to 25° C. The reaction mixture is then stirred for an additional 7 hours. The organic layer is separated, the aqueous layer is extracted with ethyl acetate (300 mL), and the combined organic layers are washed with 20% (w/v) aqueous sodium chloride (250 mL) and then dried over Na₂SO₄ (50 g). The solution is evaporated to dryness, resulting in a gummy oil (26.0 g, 89.65% crude yield). The crude gummy oil is dissolved in n-hexane (250 mL) isomerized with sodium tert-butoxide (0.78 g), as described above, to afford the product (13.0 g, 33% yield; by GC isomer (1a) 69.50%, and isomer (1b) 4.84%.)

### Example 12: 2-methyl-hexanoic acid from 2-methyl-1-hexene (One-Pot, Two Step Procedure)

To a mixture of 2-methyl-1-hexene (7.1 g, 0.072 mol) and sodium acetate (0.6 g, 0.009 mol) is added hydrogen peroxide (30% aqueous, 11.5 g, 0.101 mol) and acetic anhydride (12.6 g, 0.123 mol) dropwise over 50 min at 40° C. via separate dropping funnels. The reaction is then stirred overnight at room temperature.

Sulfuric acid (60% aqueous, 12 g) is heated in a separate flask to 70° C. The reaction mixture from the first step is then slowly added over 30 minutes, and the reaction is then refluxed for 1.5 hours, then cooled to 45° C. Additional hydrogen peroxide (30% aqueous, 6.6 g, 0.058 mol) is added over 30 minutes, and the reaction is stirred for 1.5 hours then cooled, diluted with water (50 ml), and the phases are separated. The organic layer is washed with water, 20% NaHSO₃, brine, and dried (Na₂SO₄) to afford the crude product (4.2 g, GC purity 60%, GC yield 26%).

The Examples provided herein are exemplary only and are not intended to be limiting in any way to the various aspects and embodiments of the invention described herein.

I/We claim:

1. A method of making a carboxylic acid compound (24) from an alkene compound (21), wherein the method proceeds through intermediate compounds (22) and (23), which optionally are generated in-situ and not isolated or purified: 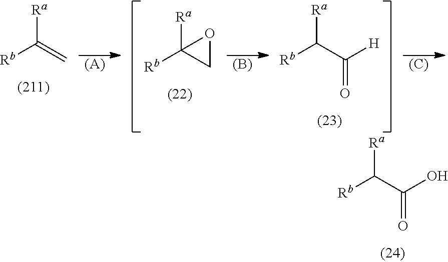
wherein the method comprises the following mechanistic steps;
(A) epoxidizing alkene Compound (21), to form epoxide Compound (22);
(B) rearranging epoxide Compound (22) to form aldehyde Compound (23); and
(C) oxidizing aldehyde Compound (23) to form carboxylic acid Compound (24);
wherein R^{a} and R^{b} are each independently selected from H, optionally substituted C₁₋₃₀ alkyl, optionally substituted C₃₋₁₀cycloalkyl, optionally substituted heterocycloalkyl, optionally substituted aryl, optionally substituted heteroaryl, —C(O)—OR^{c} and —C(O)—R^{c}, and R^{c} is selected from H, optionally substituted C₁₋₆alkyl, optionally substituted C₃₋₁₀cycloalkyl, optionally substituted heterocycloalkyl, and optionally substituted aryl;
wherein the method optionally further comprises a step (D) of esterification or lactonization of the carboxylic acid Compound (24) to form the ester or lactone Compound (25): 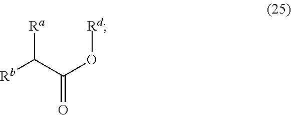
wherein R^{d} is optionally substituted C₁₋₁₂alkyl, or wherein R^{d} and either R^{a} or R^{b} together form a 5-10-membered heterocyclic ring or a C₅₋₁₀carbocyclic ring.

2. The method according to claim 1, which is a method of making a compound (1) from compound (2), or of making compound (6) from compound (7): 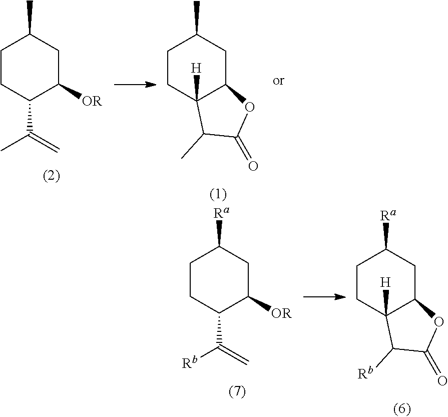
the method comprising the step of treating the compound (2) or (7) with an oxidizing agent, and optionally an acid or base, in a suitable solvent (e.g., aqueous solvent), wherein the reaction proceeds in a single vessel without the isolation of any intermediates (e.g., a one-pot reaction);
wherein R is H or a protecting group (e.g., an ether, an ester, or a silyl group), and wherein R^{a} is selected from H, optionally substituted C₁₋₆alkyl, optionally substituted C₂₋₆alkenyl, optionally substituted C₂₋₆alkynyl, optionally substituted aryl, O—R^{c}, and —C(O)—R^{c}, and R^{b} is selected from H, optionally substituted C₁₋₆alkyl, optionally substituted aryl, O—R^{c}, and —C(O)—R^{c}, and R^{c} is selected from H, optionally substituted C₁₋₆alkyl, optionally substituted C₂₋₆alkenyl, optionally substituted C₂₋₆alkynyl, and optionally substituted aryl.

3. The method according to claim 1, which is a method of making 3,6-dimethylhexahydrobenzofuran-2-one (Compound 1) comprising the steps of:
(A) epoxidizing isopulegol or a derivative thereof (Compound 2), to form epoxide Compound (3);
(B) rearranging epoxide Compound (3) to form aldehyde Compound (4);
(C) oxidizing aldehyde Compound (4) to form carboxylic acid Compound (5); and
(D) ring closing the carboxylic acid Compound (5) to form 3,6-dimethylhexahydrobenzofuran-2-one (Compound 1): 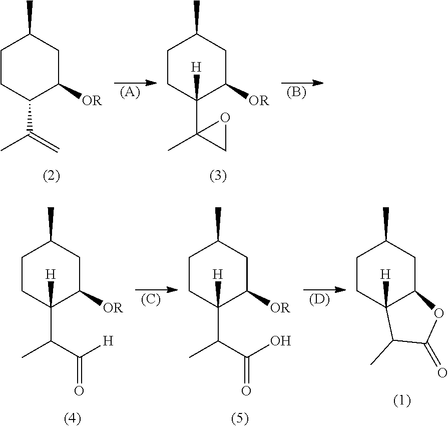
wherein R is H or a protecting group (e.g., an ether protecting group, an ester protecting group, or a silyl ether protecting group).

4. The method according to claim 2, wherein R is H.

5. The method according to claim 2, wherein R is an ester protecting group, e.g., —C(O)—R¹, and wherein R¹ is H, C₁₋₆alkyl (e.g., methyl or ethyl), haloC₁₋₆alkyl (e.g., chloromethyl or trifluoromethyl), C₁₋₆alkoxy (e.g., methoxy or ethoxy), C₁₋₆alkoxymethyl (e.g., methoxyethyl or ethoxymethyl), aryl (e.g., phenyl), arylmethyl (e.g., benzyl), aryloxy (e.g., phenoxy), or aryloxymethyl (e.g., phenoxymethyl).

6. The method according to claim 5, wherein R is —C(O)—R¹, and wherein R¹ is methyl, ethyl, propyl, isopropyl, or tert-butyl.

7. The method according to claim 5, wherein R is —C(O)—R¹, and wherein R¹ is methyl.

8. The method according to claim 3, wherein the Compound (3) is Compound (3a), Compound (3b) or a mixture thereof: 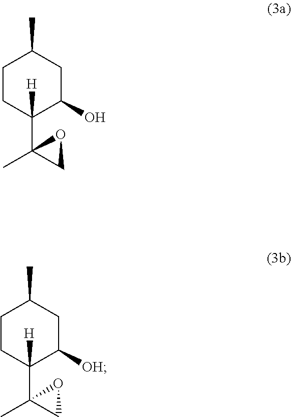
and/or
wherein the Compound (4) is Compound (4a), Compound (4b) or a mixture thereof: 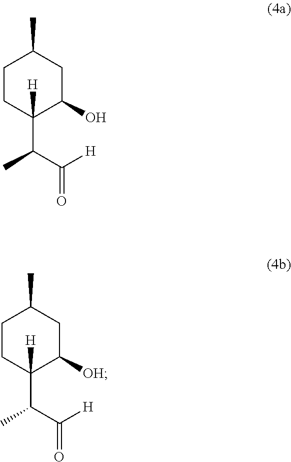
and/or
wherein the Compound (5) is Compound (5a), Compound (5b) or a mixture thereof: 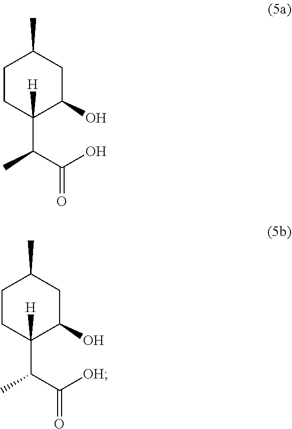
and/or
wherein the Compound (1) is Compound (1a), Compound (1b) or a mixture thereof:

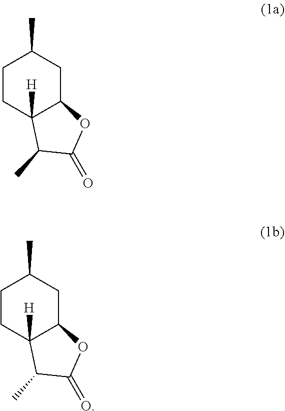

9. The method according to claim 3, wherein the method does not comprise any step using mint lactone (compound (VI) hereinabove) as an intermediate.

10. The method according to claim 3, wherein the method does not comprise any step using compound (IV), compound (V), or compound (VIII), as an intermediate: 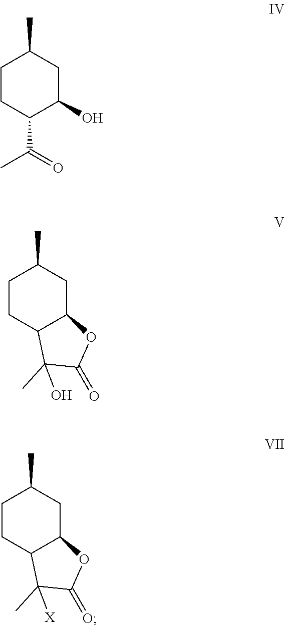
wherein X is Cl, Br, or I.

11. The method according to claim 3, wherein epoxidation step (A) is carried out by treating the compound (2) with a suitable oxidizing agent in a suitable solvent, optionally wherein the suitable oxidizing agent is one or more of hydrogen peroxide, osmium tetroxide, peracetic acid, perchloric acid, perbenzoic acid, meta-chloroperoxybenzoic acid (mCPBA), trifluoroperacetic acid, magnesium monoperoxyphthalate, dimethyl dioxirane (DMDO), tert-butyl hydroperoxide, sodium hypochlorite, sodium tungstate, sodium periodate, iodosyl benzene, pentafluoroiodosyl benzene, cumene hydroperoxide, potassium persulfate, potassium monoperoxysulfate, pyridine N-oxide, 2,6-dichloropyridine N-oxide, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries).

12. The method according to claim 3, wherein the rearrangement step (B) is carried out by treating the compound (3) with a suitable rearrangement catalyst in a suitable solvent, or by heating the compound (3) without catalyst in a suitable solvent (i.e., thermal rearrangement), optionally wherein the rearrangement catalyst is a Lewis acid, a Bronsted acid, a strong base (e.g., LDA, LiTMP, LiHMDS, t-butyl lithium), or a transition metal catalyst or complex (e.g., palladium, ruthenium, rhodium, chromium, iridium, zirconium, manganese, iron, or nickel catalyst or complex);

13. The method according to claim 12, wherein the rearrangement catalyst is a solid phase acidic resin (e.g., an acidic polymer resin such as Amberlyst or Nafion-H, or a Montmorillonite, or a Zeolite), e.g., Montmorillonite K10, or Amberlyst H-15.

14. The method according to claim 3, wherein oxidation step (C) is carried out by treating the compound (4) with a suitable oxidizing agent in a suitable solvent, optionally wherein in oxidation step (C) the suitable oxidizing agent is one or more of a chromium oxidant (e.g., chromium trioxide, chromic acid, pyridinium chlorochromate, potassium dichromate, chromium trioxide-pyridine complex, pyridinium dichromate), osmium tetroxide, potassium permanganate, silver oxide, hydrogen peroxide, peracetic acid, perchloric acid, trifluoroperacetic acid, periodic acid, potassium periodate, sodium chlorite, oxygen with N-hydroxyphthalimide, potassium persulfate, and potassium monoperoxysulfate.

15. The method according to claim 3, wherein ring closure step (D) occurs spontaneously during and/or after oxidation step (C).

16. The method according to claim 3, wherein ring closure step (D) is carried out by heating the product mixture from step (C).

17. The method according to claim 3, wherein the intermediates (3), (4), and (5), of steps (A), (B), (C), and (D) are not isolated, e.g., wherein the reactant Compound (2) proceeds to the product Compound (1) in a single vessel.

18. The method according to claim 17, wherein the reaction comprises the treatment of Compound (2) with an oxidizing agent, and an acid, in a suitable solvent.

19. The method according to claim 18, wherein the oxidizing agent is selected from one or more of hydrogen peroxide, a chromium oxidant (e.g., chromium trioxide, chromic acid, pyridinium chlorochromate, potassium dichromate, chromium trioxide-pyridine complex, pyridinium dichromate), osmium tetroxide, potassium permanganate, peracetic acid, perchloric acid, perbenzoic acid, meta-chloroperoxybenzoic acid (mCPB A), trifluoroperacetic acid, periodic acid, magnesium monoperoxyphthalate, dimethyl dioxirane (DMDO), tert-butyl hydroperoxide, sodium hypochlorite, sodium tungstate, sodium periodate, potassium periodate, iodosyl benzene, pentafluoroiodosyl benzene, cumene hydroperoxide, potassium persulfate, potassium monoperoxysulfate, pyridine N-oxide, 2,6-dichloropyridine N-oxide, sodium chlorite, sodium hypochlorite, sodium chlorate, sodium perchlorate, or oxygen (e.g., in combination with a transitional metal catalyst, e.g., an iron catalyst), optionally in combination with any secondary reagents (e.g., secondary oxidants, catalysts, complexing agents, directing agents, reducing agent, or chiral auxiliaries); and wherein the acid is a Bronsted acid, e.g., selected from hydrochloric acid, perchloric acid, sulfuric acid, phosphoric acid, acetic acid, peracetic acid, trifluoromethanesulfonic acid, methanesulfonic acid, trifluoroacetic acid, and nitric acid, or a heteropoly acid (e.g., phosphotungstic acid); and wherein the reaction is carried out in a solvent selected from hydrocarbons, chlorinated hydrocarbons, aromatics, ethers, esters, ketones, polar aprotic solvents, polar protic solvents, carbon dioxide, acetic anhydride, carbon disulfide, or a combination thereof.

20. The method according to claim 19, wherein the oxidizing agent is hydrogen peroxide, peracetic acid, trifluoroperacetic acid, meta-chloroperoxybenzoic acid, tert-butyl hydroperoxide, or potassium monoperoxysulfate; and wherein the acid is selected from sulfuric acid, phosphoric acid, and nitric acid; and wherein the solvent is acetic acid.

21. The method according to claim 3, wherein the method further comprises a Step (E) of treating the Compound (1) with a base to cause enrichment of the Compound (1a) by isomerization of the Compound (1b) to the Compound (1a).

22. The method according to claim 2, wherein the oxidizing agent is hydrogen peroxide, peracetic acid, trifluoroperacetic acid, meta-chloroperoxybenzoic acid, tert-butyl hydroperoxide, or potassium peroxymonosulfate; and wherein the acid is selected from sulfuric acid, phosphoric acid, trifluoromethanesulfonic acid, and nitric acid; and wherein the solvent is acetic acid

23. The method according to claim 1, which is a method of making an optionally 3,6-disubstituted hexahydrobenzofuran-2-one (Compound 6) comprising the steps of:
(A) epoxidizing an optionally 5-substituted-2-vinylcyclohexanol, or a derivative thereof (Compound 7), to form epoxide Compound (8);
(B) rearranging epoxide Compound (8) to form aldehyde Compound (9);
(C) oxidizing aldehyde Compound (9) to form carboxylic acid Compound (10); and
(D) ring closing the carboxylic acid Compound (10) to form the Compound (6): 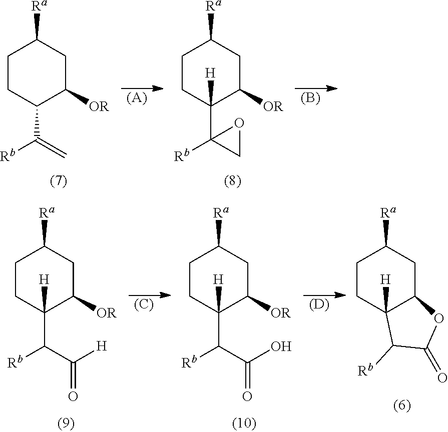
wherein R is H or a protecting group (e.g., an ether, an ester, or a silyl group), and R^{a} is selected from H, optionally substituted C₁₋₆alkyl, optionally substituted C₂₋₆alkenyl, optionally substituted C₂₋₆alkynyl, optionally substituted aryl, O—R^{c}, and —C(O)—R^{c}, and R^{b} is selected from H, optionally substituted C₁₋₆alkyl, optionally substituted aryl, O—R^{c}, and —C(O)—R^{c}, and R^{c} is selected from H, optionally substituted C₁₋₆alkyl, optionally substituted C₂₋₆alkenyl, optionally substituted C₂₋₆alkynyl, and optionally substituted aryl.
